(19) United States
(12) Patent Application Publication
(10) Pub. No.: US 2025/0295652 A1
(43) Pub. Date: Sep. 25, 2025

(54) Inhibitors of Molluscum Contagiosum Infection and Methods Using the Same

(71) Applicant: THE TRUSTEES OF THE UNIVERSITY OF PENNSYLVANIA, Philadelphia, PA (US)

(72) Inventors: Robert P. Ricciardi, Kennett Square, PA (US); Manunya Nuth, Philadelphia, PA (US); Hancheng Guan, Downingtown, PA (US); Allen B. Reitz, Lansdale, PA (US); Michael H. Parker, Chalfont, PA (US); Simon David Peter Baugh, Ringoes, NJ (US); Richard W. Scott, West Chester, PA (US); Eric Strobel, Warrington, PA (US)

(21) Appl. No.: 18/695,735

(22) PCT Filed: Sep. 27, 2022
(86) PCT No.: PCT/US2022/077058

§ 371 Date: Mar. 26, 2024

Related U.S. Application Data
(60) Provisional application No. 63/248,670, filed on Sep. 27, 2021.

(51) Int. Cl. A61K 31/496; A61K 31/381; A61K 31/4035; A61K 31/425; A61K 31/426; A61K 31/433; A61K 31/4545; A61K 31/5377; A61K 47/10; A61K 47/14; A61K 47/26; A61K 47/38; C07D 275/03; C07D 277/46; C07D 285/06; C07D 333/38; C07D 409/12; C07D 417/12

(52) U.S. Cl. CPC A61K 31/496; A61K 31/381; A61K 31/4035; A61K 31/425; A61K 31/426; A61K 31/433; A61K 31/4545; A61K 31/5377; A61K 47/10; A61K 47/14; A61K 47/26; A61K 47/38; C07D 275/03; C07D 277/46; C07D 285/06; C07D 333/38; C07D 409/12; C07D 417/12

(57) ABSTRACT

The present invention provides novel compounds, compositions and methods for treating, ameliorating, and/or preventing an orthopoxvirus infection in a subject in need thereof. In certain embodiments, the orthopoxvirus infection is caused by Molluscum contagiosum.

### CROSS-REFERENCE TO RELATED APPLICATIONS

This application claims priority under 35 U.S.C. § 119(e) to U.S. Provisional Patent Application No. 63/248,670, filed Sep. 27, 2021, which is incorporated herein by reference in its entirety.

### STATEMENT REGARDING FEDERALLY SPONSORED RESEARCH OR DEVELOPMENT

This invention was made with government support under AI125005 awarded by National Institutes of Health. The government has certain rights in the invention.

### SEQUENCE LISTING

The XML file named “046483-7338W01_Sequence_Listing.txt” created on Sep. 22, 2022, comprising 3.2 Kbytes, is hereby incorporated by reference in its entirety.

### BACKGROUND

Molluscum contagiosum (MC) is a skin disease caused by the poxvirus Molluscum contagiosum virus (MCV). MC presents as skin lesions that can last from months to years before resolving. MC lesions occur in children, adults, and immunosuppressed individuals, and are restricted strictly to the skin. MCV is transmitted by direct skin-to-skin contact, sexual contact, auto-inoculation from scratching lesions, and by indirect inoculation from contaminated fomites. The lesions can be painful following treatments intended to reduce spread. The lesions are also psychologically distressful, even more so when they result in scarring. MC occurs in 2-10% of the worldwide population and in the U.S., it constitutes about 1% of all diagnosed skin disorders, and in children it approaches 5%. In immunocompromised individuals, this infectious disease can be both severe and protracted. Between 5% and 18% of HIV patients have MC. Often, severe MC disease in AIDS patients begins to resolve while on highly active antiretroviral therapy (HAART). However, there have been documented cases of MC lesions developing soon after starting HAART, suggesting that immune reconstitution inflammatory syndrome (IRIS) might be playing a role in re-emergence of MCV.

The current treatments for MC usually employ physical therapy or chemical agents, which are not uniformly effective or safe, and often fail to completely eliminate lesions and may result in scaring. In addition, the broad-spectrum antiviral drug cidofovir (or 1-((3-hydroxy-2-phosphonyl methoxy)propyl)cytosine), a dCMP analogue, has been used effectively as topical or intravenous medication for MC in immunocompromised patients. However, this drug has side effects including inflammation, erosion and pain for topical treatment and potential nephrotoxicity for systemic application. To date, no single antiviral therapeutic has been licensed for the specific treatment of MC. The development of such an effective and safe treatment has been hampered mainly by the inability of MCV to propagate in culture.

Processivity factors (PFs) are attractive antiviral therapeutic targets. Their function is to tether DNA polymerases (Pol) to the template to enable synthesis of extended strands. PFs are specific for their cognate DNA Pol and are absolutely essential for DNA synthesis. All DNA Pols from phage to human function with a single cognate PF. However the poxviruses, including the prototypic vaccinia virus (VV) and MCV, are somewhat unusual in that a heterodimer comprising the A20 and D4 viral proteins constitutes the functional PF. D4, which can also function as a uracil-DNA glycosylase repair enzyme, binds to its PF partner A20 but not to E9 Pol. A20 on the other hand, binds to both E9 and D4, suggesting that it serves, in part, as a bridge that indirectly connects D4 to E9.

D4 is also an attractive antiviral target due to its absolute requirement for DNA synthesis by both MCV and VV (the prototypic poxvirus). Notably, in the in vitro DNA synthesis reaction, MCV D4 (mD4) can equally substitute for VV D4 (vD4). This is consistent with mD4 having an amino acid sequence identity of 55% and similarity of 82% to that of VV. Moreover, the virtual 3-D structure of mD4 superimposes onto the known crystal structure of vD4.

There is thus a need in the art for identifying novel compounds and formulations that can be used to treat and/or prevent MC infections in humans. In certain embodiments, such compounds and compositions should be formulated for topical or intradermal administration. The present invention addresses this need.

### BRIEF SUMMARY

The present disclosure provides, in one aspect, a compound of formula (I), or a salt, solvate, enantiomer, diastereoisomer, geometric isomer, or tautomer thereof:

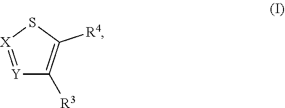

wherein X, Y, R³, and R⁴ are defined within the scope of the present disclosure.

In another aspect, the present disclosure provides a pharmaceutical composition comprising at least one compound of Formula (I) and a pharmaceutically acceptable excipient. In certain embodiments, the at least one pharmaceutically acceptable excipient is at least one selected from the group consisting of water, polyethylene glycol (PEG) 400, PEG 300, propylene glycol (PG), benzyl alcohol, polysorbate 80, diethylene glycol monoethyl ether (DEGEE), isopropyl myristate, ethanol, diisopropyl adipate, C₁₂₋₁₅ alkyl lactate, thickening agent, hydroxypropyl cellulose, and PEG 4000.

In certain embodiments, the pharmaceutical composition is formulated for topical administration. In certain embodiments, the topical administration comprises a gel or ointment

In another aspect, the present disclosure provides a method of treating, ameliorating, and/or preventing an orthopoxvirus infection in a human subject in need thereof. In certain embodiments, the method comprises administering to the subject a therapeutically effective amount of at least one pharmaceutical composition of the present disclosure, or a compound of formula (II):

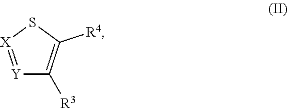

wherein X, Y, R³, and R⁴ are defined within the scope of the present disclosure.

In certain embodiments, the orthopoxvirus infection is caused by a virus selected from the group consisting of Molluscum contagiosum virus (MCV), amelpox virus, cowpox virus, mousepox virus, horsepox virus, monkeypox virus, raccoonpox virus, tanapox virus, varioloa (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus). In certain embodiments, the orthopoxvirus infection is caused by a Molluscum contagiosum virus (MCV).

In certain embodiments, the compound or composition is applied to the skin of the subject.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1A illustrates interactions of poxvirus 4 processivity factor with its A20 partner (the Processivity Factor complex) and the DNA polymerase and the DNA template. FIG. 1B illustrates requirement for D4 in DNA synthesis. The model also suggests non-limiting relevant interactions of the E9, A20, and D4 triad. The poxvirus processivity factor complex (D4 and A20) associates with its cognate DNA polymerase (E9) and tethers it to the DNA. This enables E9 Pol to synthesize extended strands by incorporating nucleotides continuously. In the absence of the processivity factor complex, the catalytic activity of E9 Pol is insufficient to synthesize extended strands.

FIG. 2A illustrates alignment of mD4 (SEQ ID NO:1) and vD4 (SEQ ID NO:2). Identical amino acids are shaded. Missing amino acids are denoted by dashes. The C-terminal residues 167-180 and 191-206 of vD4 (underlines) are important for interacting with A20. FIG. 2B illustrates superimposition of mD4 predicted structure onto vD4 crystal structure. mD4 structure was generated by homology modeling using the SWISS-MODEL.

FIG. 3A comprises a graph illustrating the finding that Compound 1 binds to D4 when assayed by DSF (Differential Scanning Fluorimetry). FIG. 3B illustrates that negative controls Cidofovir and ST-246 (Tecovirimat), as compared to Compound 1, do not exhibit binding to D4. SPR data for FIG. 3B: upper left, dose-dependent binding of Compound 1 to D4; upper right, calculation of dissociation constant of binding Compound 1 to D4; lower left and right, negative controls showing CDV and ST-246 fail to bind D4.

FIG. 4 comprises images relating to Drug Affinity Responsive Target Stability DARTS studies, demonstrating that exemplary Compound 1 binds to the D4 target protein as evidenced by protection against proteolysis.

FIG. 5 illustrates Processivity Factor—Dependent DNA Synthesis Assay. The illustration depicts the assay used to evaluate activity of Molluscum D4 processivity protein (mD4) for DNA synthesis. Each individual reaction contains all of the components necessary to permit processive DNA synthesis of non-radioactive DNA. In this assay, a 100-nucleotide template contains a biotin moiety on its 5′ end and a 15-nucleotide primer annealed to its 3′ end. The annealed primer template is attached to streptavidin-coated wells of a 96-well plate. An oligonucleotide primer (15mer) is annealed to the 3′ end of the biotin-labeled oligonucleotide template. Addition of DNA Pol, D4 and A20 enables incorporation of dNTPs and dig-dUTP that is recognized by digoxigenin (DIG) antibody coupled to HRP for colorimetric quantitation (405 nm). In the presence of a D4 antiviral therapeutic compound (not shown), nucleotide incorporation fails and DNA synthesis does not occur as determined by lack of colorimetric detection. This assay has been fully described (Lin & Ricciardi, 2000, J. Virol Methods 88:219-225; U.S. Pat. No. 6,204,028, which is incorporated herein in its entirety by reference).

FIG. 6 comprises a graph illustrating that Compound 1 blocks mD4-dependent processive DNA synthesis in vitro in the Processivity Factor—Dependent DNA Synthesis Assay.

FIG. 7 comprises a graph illustrating that Compound 1 blocks mD4 hybrid-poxvirus infection (potency expressed as EC₅₀; upper image), and actual viral plaque reduction (lower image). Tissue culture plates of stained cells—clear circles on the left plate are infection centers of cells destroyed in the presence of DMSO carrier (control) and solid staining on the right plate represent plates protected by Compound 1.

FIGS. 8A-8B comprise graphs demonstrating that compounds of the invention bind to D4 with specificity. FIG. 8A comprises a graph illustrating that Compound 1 does not block Herpes Simplex Virus-1 (HSV-1) DNA synthesis. FIG. 8B comprises a graph illustrating that Compound 1 does not block Herpes Simplex Virus-1 (HSV-1) ability to infect cells.

FIG. 9 comprises a chart summarizing biological activity for selected compounds of the invention. Those compounds were found to target mD4 and specifically block mD4 dependent processive DNA synthesis and infection of the Molluscum mD4 hybrid-virus. Similar results were obtained for Vaccinia Virus.

FIGS. 10A-10B comprise graphs that reveal protein dynamic features of D4 with respect to (FIG. 10A) tryptophan fluorescence and (FIG. 10B) sensitivity to proteolysis. FIG. 10A: Tryptophan fluorescence of D4 and MBP in presence of the indicated concentrations of DMSO. The insets depict different spectra of proteins in absence or presence of the highest concentrations of DMSO studied. FIG. 10B: Sensitivity to proteolysis. Pronase was prepared as a 10 mg/ml stock and diluted accordingly. Proteins are depicted with their corresponding tags for probing by Western blotting. His-tagged D4 (˜27 kDa) was probed with anti-His antibody, while MBP fusions of human estrogen receptor beta (ERβ-N; ˜54 kDa), MBP (introduced with a C-terminal Hiss; ˜44 kDa), and the N-terminal 63-aa acid stretch of A20 (A20₆₃; ˜52 kDa) were probed with anti-MBP antibody.

FIGS. 11A-11C comprise graphs illustrating certain compound selection tests. FIG. 11A: Evaluation for ability to inhibit in vitro DNA synthesis at a single dose of 500 μM. FIG. 11B: Screening selected compound for their abilities to block VACV infection at a single dose of 50 μM. FIG. 11C: Screening compounds that block viral infection (≥50% antiviral activity) for their abilities to block processive DNA synthesis.

FIGS. 12A-12D comprises graphs that assess compound binding to D4 based on (FIG. 12A) DSF, (FIG. 12B) SPR, and (FIGS. 12C-12D) DARTS. FIG. 12B: For SPR, the sensor chip NTA was crosslinked with MBP onto the reference flow cell and D4 onto the active flow cell. R_{eq}=response from steady-state, shown as dashed lines in the sensogram overlay; CDV=cidofovir; and ST-246=tecovirimat. FIG. 12C: Binding of Compound 1 to various proteins as measured by DARTS. Compound 1 was incubated with diluted crude lysates expressing the indicated proteins, and proteolysis was achieved by addition of Pronase at 1:37.5 dilution for determination of MBP, 1:150 for A20₆₃, 1:300 for D4, and 1:2400 for ERβ-N. FIG. 12D: Binding of poxvirus drugs to D4 as measured by DARTS. Pronase was used at 1:300 dilution.

FIG. 13 illustrates heat traces to evaluate binding of Compound 1 to DNA. Heat traces are shown with the use of random duplex DNA (sheared DNA, shown resolved on 1% agarose gel) or single-stranded DNA with the use of d(TC) 15-mer (*). Heat traces are rescaled in order to permit comparison, and uncorrected heats (Q) are shown without further deconvolution.

FIGS. 14A-14D shows inhibition of DNA synthesis by compounds 29 (FIG. 14A), 99 (FIG. 14B), 186 (FIG. 14C), and 187 (FIG. 14D) in the in vitro mD4-dependent processive DNA synthesis assay

FIGS. 15A-15D shows inhibition of mD4-VV DNA infection by compounds 29 (FIG. 15A), 99 (FIG. 15B), 186 (FIG. 15C), and 187 (FIG. 15D). mD4-VV surrogate virus infected BSC-1 cells were treated with increasing amounts of each compound and viral plaques were quantitated 24 h post infection. As a control, HSV1 plaque formation in the presence of the compounds was analyzed at 53 h post infection on Vero cells. The data represents mean SD of plaque numbers from at least two independent experiments in duplicate.

FIG. 16 provides several compounds of the present disclosure and corresponding biological activities and/or physical properties.

FIGS. 17A-17B display the average cumulative amounts of compound recovered in each of the samples, including stratum corneum, epidermis, dermis and receptor media. FIG. 17A: average cumulative amount of compound 111 released. FIG. 17B: average cumulative amount of compound 99 released.

FIGS. 18A-18B provide average epidermal and dermal skin concentrations observed during an in vitro skin penetration study of compounds 111 (FIG. 18A) and 99 (FIG. 18B).

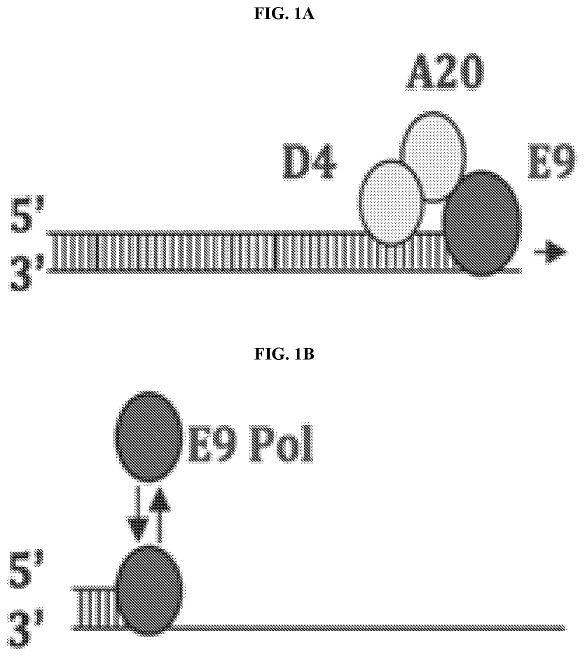

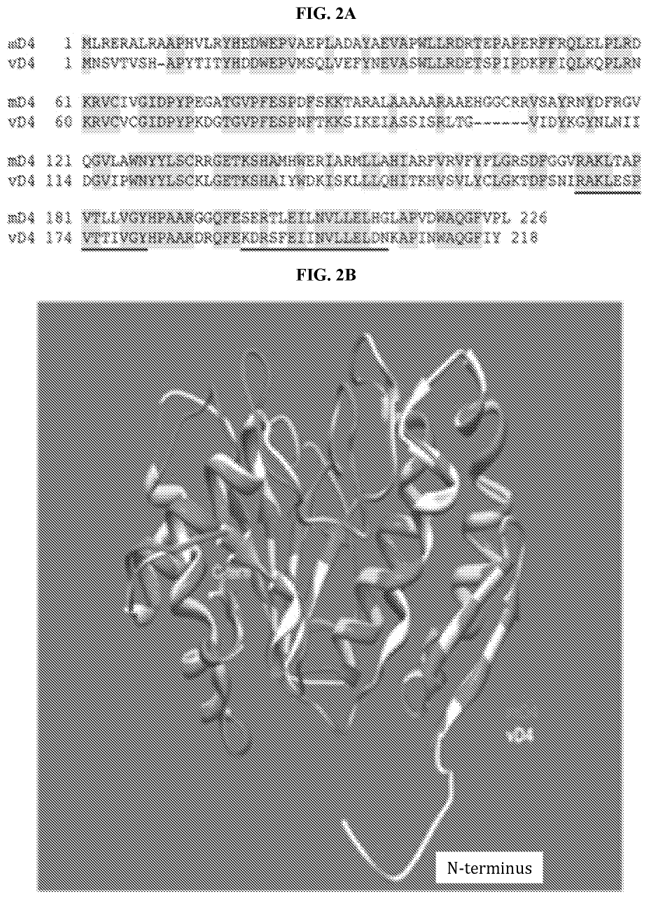

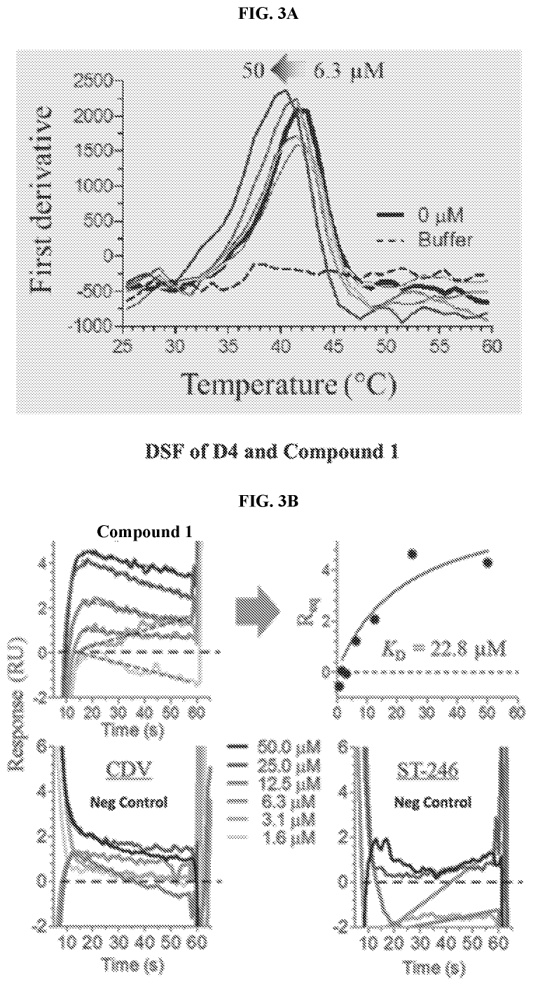

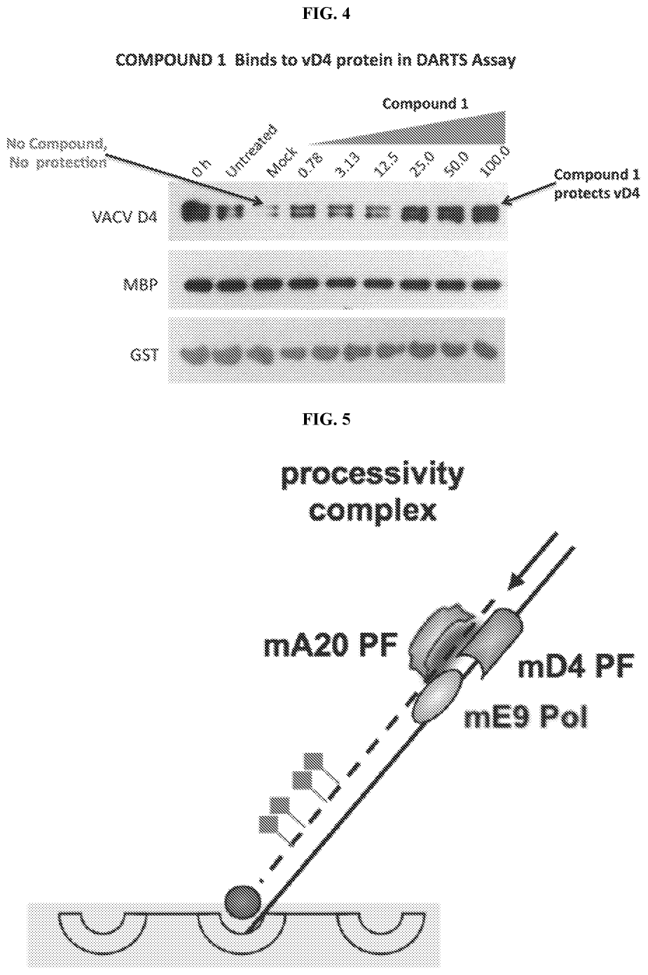

### DETAILED DESCRIPTION

The present invention relates in part to the unexpected discovery of novel inhibitors of Molluscum contagiosum virus (MCV) infection in a human. Molluscum contagiosum virus (MCV) infects humans only. In humans, the virus infection is confined to the skin and is not systemic. In certain embodiments, all the inhibitors described herein also block vaccinia, the prototypic poxvirus. In other embodiments, other poxviruses such as, but not limited to camelpox virus, cowpox virus, ectromelia virus, horsepox virus, monkeypox virus, racoonpox virus, turkeypoxvirus, variola smallpox virus, Yoka poxvirus, deer poxvirus, fowl poxvirus, myxoma virus, Orf virus, swinepox virus, and Yaba-like disease virus can be inhibited the compounds described herein.

In certain embodiments, the compounds of the invention, or any compositions comprising the same, treat, prevent, and/or ameliorate MCV infection when applied to the skin of an infected human. In yet other embodiments, the compounds of the invention, or any compositions comprising the same, are applied to at least one MCV lesion on the skin of the infected human.

The poxvirus D4 processivity factor is essential for viral replication. The viral D4 and A20 proteins form a complex that serves to tether to the viral polymerase to the template, enabling it to synthesize long-extended strands of DNA. Processivity factors are compelling drug targets based on specificity for their cognate DNA polymerases.

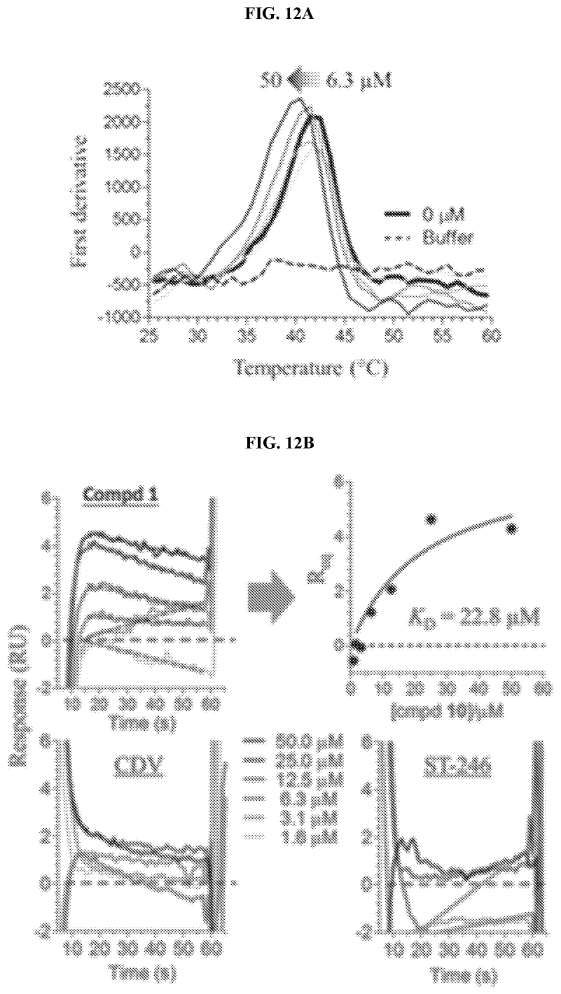

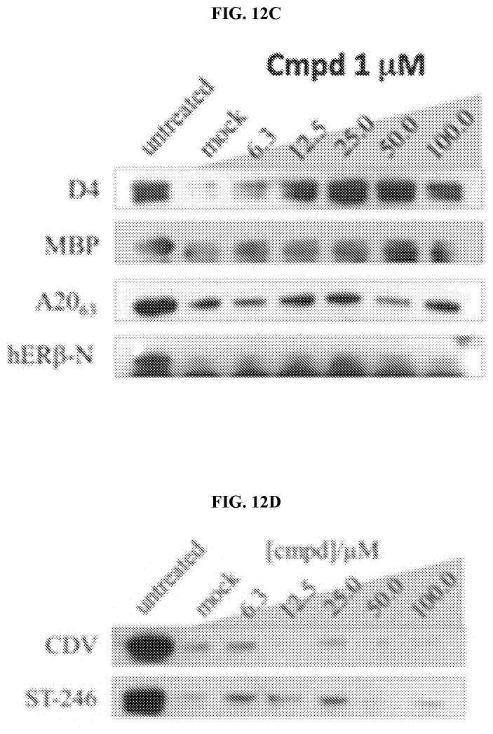

Interaction of Compound 1 with D4 protein was demonstrated by three independent biophysical measurements: DARTS (Drug Affinity Responsive Target Stability); DSF (Differential Scanning Fluorimetry); and SPR (Surface Plasma Resonance; FIGS. 12A-12D).

Together, these biophysical studies reveal that binding of D4 by Compound 1 leads to protein destabilization, which as a consequence prevents processive DNA synthesis in vivo, and prevents poxvirus from infecting cells in culture.

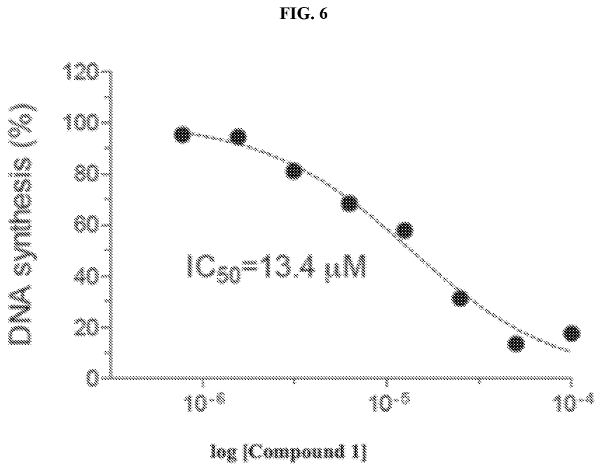

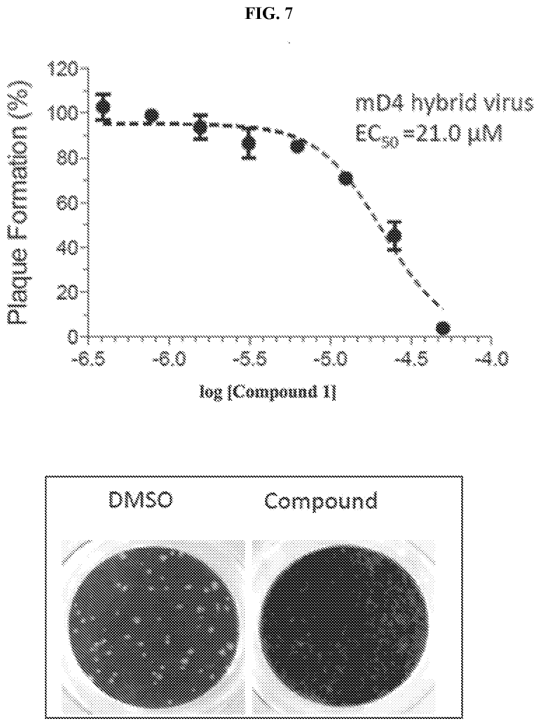

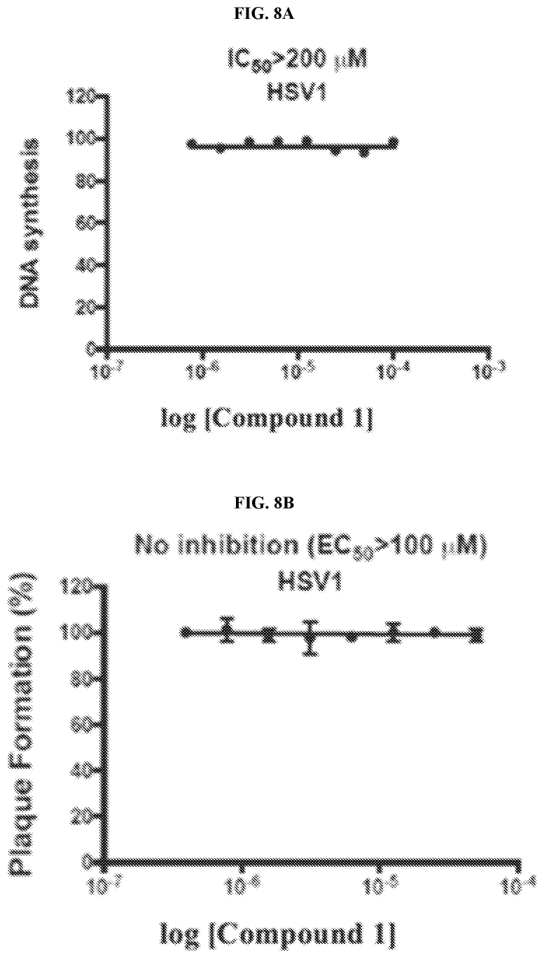

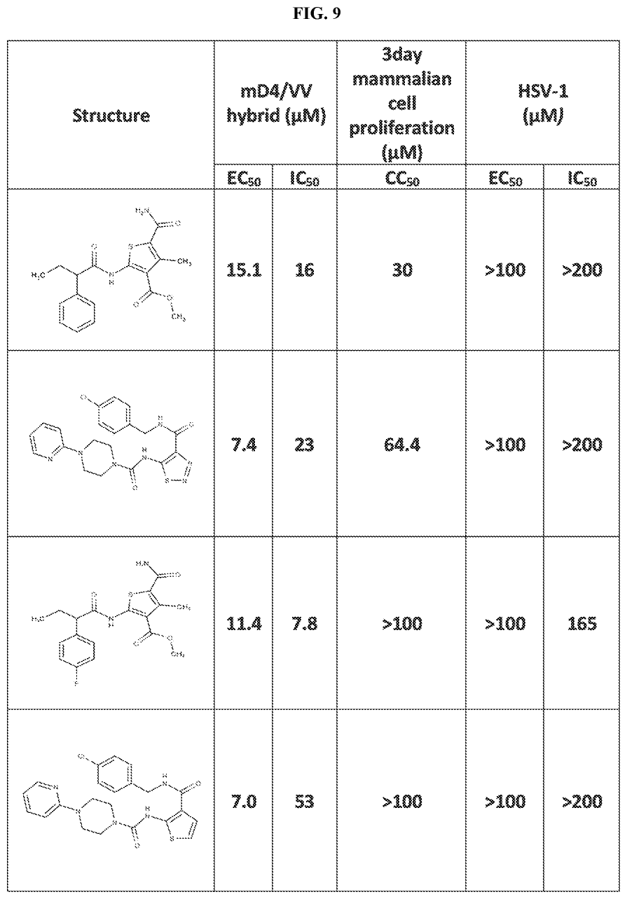

An in vitro processivity DNA synthesis assay (Lin &Ricciardi, 2000, J. Virol Methods 88:219-225; U.S. Pat. No. 6,204,028) (FIG. 5) demonstrated that Compound 1 is able to block molluscum mD4-dependent processive DNA synthesis (FIG. 6). Compound 1 was shown to be capable of blocking poxvirus infection in a standard cellular Plaque Reduction Assay (FIG. 7). Compound 1 was further shown to demonstrate specificity as it was unable to block Herpes Simplex Virus-1 (HSV-1) processive DNA synthesis (FIG. 8A) and was also unable to block HSV-1 infection (FIG. 8B). Compound 1 binding to D4 disables viral DNA synthesis and viral infection. Additional analogs (Compounds 2-4) were synthesized and analyzed for inhibition of mD4-dependent DNA synthesis (IC₅₀); infection (IE₅₀); cell-proliferation and specificity (failure to block HSV-1).

### Definitions

As used herein, each of the following terms has the meaning associated with it in this section.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

Generally, the nomenclature used herein and the laboratory procedures in pharmaceutical science and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or ±10%, in certain other embodiments±5%, in other embodiments±1%, and in yet other embodiments±0.1% from the specified value, as such variations are appropriate to perform the disclosed methods.

As used herein, the term “D4” refers to D4 processivity factor. Further, as used herein, the term “mD4” refers to Molluscum D4 processivity factor.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “ED₅₀” or “ED₅₀” refers to the effective dose of a formulation that produces about 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition.

As used herein, a “patient” or “subject” may be a human or non-human mammal or a bird. Non-human mammals include, for example, livestock and pets, such as ovine, bovine, porcine, canine, feline and murine mammals. In certain other embodiments, the subject is human.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention.

Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “solvate,” as used herein, refers to a compound formed by solvation, which is a process of attraction and association of molecules of a solvent with molecules or ions of a solute. As molecules or ions of a solute dissolve in a solvent, they spread out and become surrounded by solvent molecules.

The term “treat,” “treating” or “treatment,” as used herein, means reducing the frequency or severity with which symptoms of a disease or condition are experienced by a subject by virtue of administering an agent or compound to the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C3-C6)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “alkenylene”, employed alone or in combination with other terms, means, unless otherwise stated, a stable mono-unsaturated or di-unsaturated straight chain or branched chain hydrocarbon group having the stated number of carbon atoms wherein the group has two open valencies.

As used herein, the term “alkynylene”, employed alone or in combination with other terms, means, unless otherwise stated, a stable straight chain or branched chain hydrocarbon group with a triple carbon-carbon bond, having the stated number of carbon atoms wherein the group has two open valencies.

As used herein, the term “substituted alkyl”, “substituted cycloalkyl”, “substituted alkenyl”, “substituted alkynyl”, “substituted alkylene”, “substituted alkenylene”, “substituted alkynylene”, “substituted heteroalkyl”, “substituted heteroalkenyl”, “substituted heteroalkynyl”, “substituted aryl”, “substituted heteroaryl” or “substituted heterocycloalkyl” means alkyl, cycloalkyl, alkenyl, alkynyl, alkylene, alkenylene, alkynylene, heteroalkyl, heteroalkenyl, heteroalkynyl, aryl, heteroaryl, or heterocycloalkyl as defined above, substituted by one, two or three substituents selected from the group consisting of C₁-C₁₀ alkyl, halogen, perhaloakyl, ═O, —OH, alkoxy, tetrahydro-2-H-pyranyl, —NH₂, —N(CH₃)₂, phenyl, benzyl, (1-methyl-imidazol-2-yl), pyridin-2-yl, pyridin-3-yl, pyridin-4-yl, —C(═O)OH, trifluoromethyl, —C—N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain other embodiments, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3 dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles include indolyl (such as, but not limited to, 3-, 4-5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group. Non-limiting examples of “substituted” groups include C₁-C₁₀ alkyl, halogen, perhaloakyl, ═O, —OH, alkoxy, —NH₂, —N(CH₃)₂, phenyl, benzyl, (1-methyl-imidazol-2-yl), pyridin-2-yl, pyridin-3-yl, pyridin-4-yl, —C(═O)OH, —C—N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain other embodiments, the substituents vary in number between one and four. In other embodiments, the substituents vary in number between one and three. In yet other embodiments, the substituents vary in number between one and two. In yet other embodiments, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred. The term “substituted heterocycle” and “substituted heteroaryl” as used herein refers to a heterocycle or heteroaryl group having one or more substituents including halogen, CN, OH, NO₂, amino, alkyl, cycloalkyl, carboxyalkyl (C(O)Oalkyl), trifluoroalkyl such as CF₃, aryloxy, alkoxy, aryl, or heteroaryl. A substituted heterocycle or heteroaryl group may have 1, 2, 3, or 4 substituents.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The invention includes a compound of formula (I), or a salt, solvate, enantiomer, diastereoisomer, geometric isomer, or tautomer thereof:

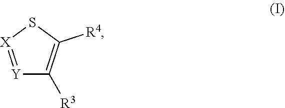

wherein:
X is CR¹ or N;
Y is CR² or N;
R¹ is H, optionally substituted C₁-C₆ alkyl (including optionally substituted benzyl), —C(═O)NR⁶R⁶, —C(═O)OR⁶, or R⁸;
R² is H or optionally substituted C₁-C₆ alkyl (including optionally substituted benzyl);
R³ is H, —CN, —C(═O)OR⁶, —C(═O)NR⁶R⁶, optionally substituted phenyl, or optionally substituted C₁-C₆ alkyl (including optionally substituted benzyl);
R⁴ is —C(═O)OR⁶ or R⁸;
each occurrence of R⁵ is independently optionally substituted C₁-C₆ alkyl (including optionally substituted benzyl), or optionally substituted phenyl;
each occurrence of R⁶ is independently H or optionally substituted C₁-C₆ alkyl (including optionally substituted benzyl), or two R⁶ bound to the same N form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷;
each occurrence of R⁷ is independently optionally substituted C₁-C₆ alkyl (including optionally substituted benzyl), optionally substituted cycloalkyl, CH (optionally substituted heterocyclyl)(R⁵), CH(R⁵)(R⁵), or optionally substituted 4-7 membered heterocyclyl,
   or R⁶ and R⁷ combine with the N atom to which both are bound to form optionally substituted 4-7 membered heterocyclyl;
with the proviso that (I) comprises a single R⁸.

In certain embodiments, the compound of formula (I) is selected from the group consisting of:

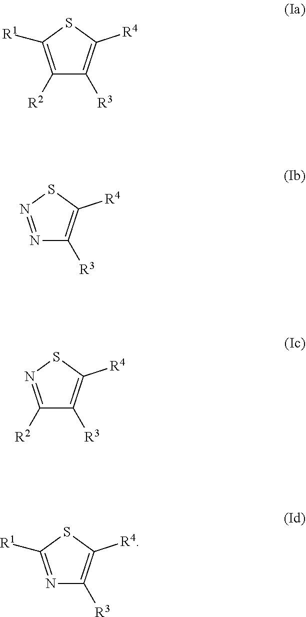

In certain embodiments, R⁴ is R⁸.

In certain embodiments, R⁸ is —NR⁶C(═O)R⁷, R⁶ is H and R⁷ is CH(CH₂CH₃)Ph.

In certain embodiments, R⁸ is —NR⁶C(═O)R⁷, R⁶ is H and R⁷ is CH(CH₂CH₃)(4-F-Ph).

In certain embodiments, R⁸ is —NR⁶C(═O)NR⁶R⁷, R⁶ is H and NR⁶R⁷=4-phenyl-piperazine-1-yl.

In certain embodiments, R⁸ is —NR⁶C(═O)NR⁶R⁷, R⁶ is H and NR⁶R⁷ is 4-(2-pyridyl)-piperazine-1-yl.

In certain embodiments, R³ is —C(═O)OR⁶ and R⁶ is CH₃.

In certain embodiments, R³ is —C(═O)OR⁶ and R⁶ is CH₂CH₃.

In certain embodiments, R³ is —C(═O)NR⁶R⁶, wherein NR⁶R⁶ is NH(CH₂-aryl), wherein the aryl is selected from the group consisting of phenyl, 4-fluorophenyl, 4-chlorophenyl, and 4-trifluoromethylphenyl.

In certain embodiments, R¹ is selected from the group consisting of:

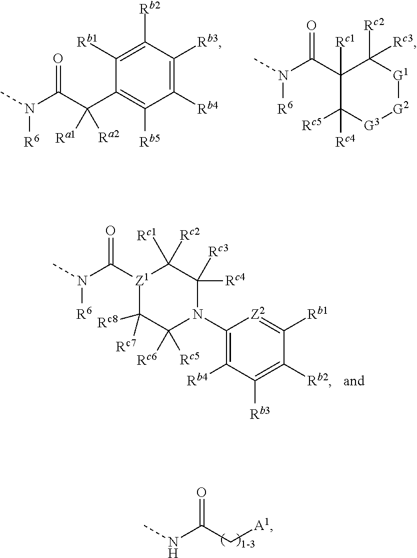

wherein:
R^{a1} and R^{a2}, if present, are each independently selected from the group consisting of H and optionally substituted C₁-C₆ alkyl;
R^{b1}, R^{b2}, R^{b3}, R^{b4}, and R^{b5}, if present, are each independently selected from the group consisting of H, optionally substituted C₁-C₆ alkyl, optionally substituted C₁-C₆ alkoxy, optionally substituted C₁-C₆ haloalkyl, halogen, CN, and NO₂,
   wherein two vicinal substituents selected from the group consisting of R^{b1}, R^{b2}, R^{b3}, R^{b4}, and R^{b5} can combine with the atoms to which they are bound to form an optionally substituted C₂-C₁₀ heterocycloalkyl;
R^{c1}, R^{c2}, R^{c3}, R^{c4}, R^{c5}, R^{c6}, R^{c7}, R^{c8}, R^{c9}, R^{c10}, and R^{c11}, if present, are each independently selected from the group consisting of H, optionally substituted C₁-C₆ alkyl, optionally substituted C₁-C₆ alkoxy, optionally substituted C₁-C₆ haloalkyl, halogen,
   wherein two vicinal substituents selected from the group consisting of R^{c1}, R^{c2}, R^{c3}, R^{c4}, R^{c5}, R^{c6}, R^{c7}, R^{c8}, R^{c9}, R^{c10}, and R¹¹ can combine with the atoms to which they are bound to form an optionally substituted C₆-C₁₀ aryl;
A¹ is selected from the group consisting of optionally substituted C₂-C₁₀ heteroaryl and optionally substituted C₂-C₁₀ heterocycloalkyl;
G¹ is selected from the group consisting of a bond and C(R^{c6})(R^{c7});
G² is selected from the group consisting of a bond and C(R^{c8})(R^{c9});
G³ is selected from the group consisting of a bond and C(R^{c10})(R^{c11});
Z¹ is selected from the group consisting of N and CR^{c9}; and
Z² is selected from the group consisting of N and CR^{b5}.

In certain embodiments, R⁴ is selected from the group consisting of:

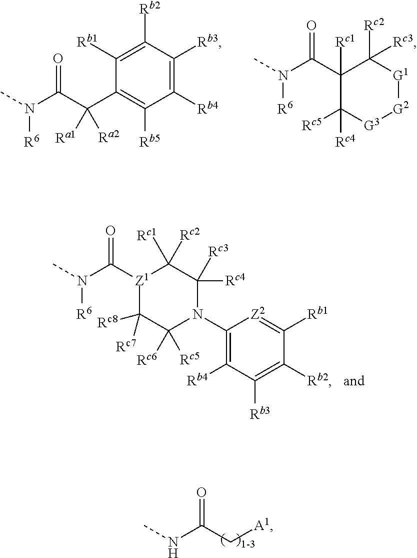

wherein:
R^{a1} and R^{a2}, if present, are each independently selected from the group consisting of H and optionally substituted C₁-C₆ alkyl;
R^{b1}, R^{b2}, R^{b3}, R^{b4}, and R^{b5}, if present, are each independently selected from the group consisting of H, optionally substituted C₁-C₆ alkyl, optionally substituted C₁-C₆ alkoxy, optionally substituted C₁-C₆ haloalkyl, halogen, CN, and NO₂,
   wherein two vicinal substituents selected from the group consisting of R^{b1}, R^{b2}, R^{b3}, R^{b4}, and R^{b5} can combine with the atoms to which they are bound to form an optionally substituted C₂-C₁₀ heterocycloalkyl;
R^{c1}, R^{c2}, R^{c3}, R^{c4}, R^{c5}, R^{c6}, R^{c7}, R^{c8}, R^{c9}, R^{c10}, and R^{c11}, if present, are each independently selected from the group consisting of H, optionally substituted C₁-C₆ alkyl, optionally substituted C₁-C₆ alkoxy, optionally substituted C₁-C₆ haloalkyl, halogen,
   wherein two vicinal substituents selected from the group consisting of R, R^{c2}, R^{c3}, R^{c4}, R^{c5}, R^{c6}, R^{c7}, R^{c8}, R^{c9}, R^{c10}, and R¹¹ can combine with the atoms to which they are bound to form an optionally substituted C₆-C₁₀ aryl;
A¹ is selected from the group consisting of optionally substituted C₂-C₁₀ heteroaryl and optionally substituted C₂-C₁₀ heterocycloalkyl;
G¹ is selected from the group consisting of a bond and C(R^{c6})(R^{c7});
G² is selected from the group consisting of a bond and C(R^{c8})(R^{c9});
G³ is selected from the group consisting of a bond and C(R^{c10})(R^{c11});
Z¹ is selected from the group consisting of N and CR^{c9}; and
Z² is selected from the group consisting of N and CR^{b5}.

In certain embodiments, R^{a1} is H and R^{a2} is ethyl. In certain embodiments, Ra is ethyl and R² is H.

In certain embodiments, R^{b1} is H. In certain embodiments, R^{b1} is methyl. In certain embodiments, R^{b1} is OMe. In certain embodiments, R^{b1} is F. In certain embodiments, R^{b1} is Cl. In certain embodiments, R^{b1} is CF₃. In certain embodiments, R^{b1} is CN. In certain embodiments, R^{b1} is NO₂.

In certain embodiments, R^{b2} is H. In certain embodiments, R^{b2} is methyl. In certain embodiments, R^{b2} is OMe. In certain embodiments, R^{b2} is F. In certain embodiments, R^{b2} is Cl. In certain embodiments, R^{b2} is CF₃. In certain embodiments, R^{b2} is CN. In certain embodiments, R^{b2} is NO₂.

In certain embodiments, R^{b3} is H. In certain embodiments, R^{b3} is methyl. In certain embodiments, R^{b3} is OMe. In certain embodiments, R^{b3} is F. In certain embodiments, R^{b3} is Cl. In certain embodiments, R^{b3} is CF₃. In certain embodiments, R^{b3} is CN. In certain embodiments, R^{b3} is NO₂.

In certain embodiments, R^{b4} is H. In certain embodiments, R^{b4} is methyl. In certain embodiments, R^{b4} is OMe. In certain embodiments, R^{b4} is F. In certain embodiments, R^{b4} is Cl. In certain embodiments, R^{b4} is CF₃. In certain embodiments, R^{b4} is CN. In certain embodiments, R^{b4} is NO₂.

In certain embodiments, R^{b5} is H. In certain embodiments, R^{b5} is methyl. In certain embodiments, R^{b5} is OMe. In certain embodiments, R^{b5} is F. In certain embodiments, R^{b5} is Cl. In certain embodiments, R^{b5} is CF₃. In certain embodiments, R^{b5} is CN. In certain embodiments, R^{b5} is NO₂.

In certain embodiments, two vicinal substituents selected from the group consisting of R^{b1}, R^{b2}, R^{b3}, R^{b4}, and R^{b5} combine to form a methylenedioxy.

In certain embodiments, R^{c1} is H. In certain embodiments, R^{c1} is Ph. In certain embodiments, R^{c2} is H. In certain embodiments, R^{c2} is Ph. In certain embodiments, R^{c3} is H. In certain embodiments, R^{c3} is Ph. In certain embodiments, R^{c4} is H. In certain embodiments, R^{c4} is Ph. In certain embodiments, R^{c5} is H. In certain embodiments, R^{c5} is Ph. In certain embodiments, R^{c6} is H. In certain embodiments, R^{c6} is Ph. In certain embodiments, R^{c7} is H. In certain embodiments, R^{c7} is Ph. In certain embodiments, R^{c8} is H. In certain embodiments, R⁸ is Ph. In certain embodiments, R^{c9} is H. In certain embodiments, R^{c9} is Ph. In certain embodiments, R^{c10} is H. In certain embodiments, R^{c10} is Ph. In certain embodiments, R^{c11} is H. In certain embodiments, R^{c11} is Ph.

In certain embodiments, two vicinal substituents selected from the group consisting of R^{c1}, R^{c2}, R^{c3}, R^{c4}, R^{c5}, R^{c6}, R^{c7}, R^{c8}, R^{c9}, R^{c10}, and R^{c11} combine to form a fused-phenyl.

In certain embodiments, none of G¹, G², and G³ is a bond. In certain embodiments, one of G¹, G², and G³ is a bond. In certain embodiments, two of G¹, G², and G³ are a bond.

In certain embodiments, each of G¹, G², and G³ is a bond.

In certain embodiments, A¹ is

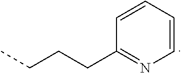

In certain embodiments, A¹ is

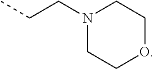

In certain embodiments, R² is optionally substituted C₁-C₆ alkyl, R³ is C(═O)OR⁶, one of R¹ and R⁴ is

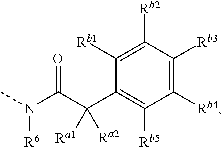

and at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy.

In certain embodiments, R³ is optionally substituted C₁-C₆ alkyl, R² is C(═O)OR⁶, one of R¹ and R⁴ is

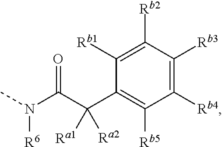

and at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy.

In certain embodiments, R² is optionally substituted C₁-C₆ alkyl, R³ is CN, one of R¹ and R⁴ is

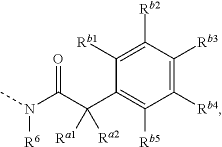

and at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy.

In certain embodiments, R² is C(═O)OR⁶, one of R¹ and R⁴ is

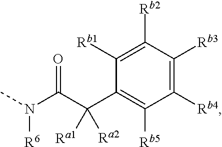

and no more than one of R³ and R¹ or R⁴ is C₁-C₆ alkyl.

In certain embodiments, R¹ is H, R² is H, and R³ is —C(═O)NR⁶R⁶, and no more than one occurrence of R⁶ is H.

In certain embodiments, R¹ is

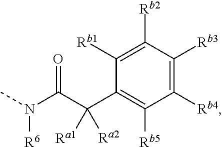

R² is methyl, R³ is H, and R⁴ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl.

In certain embodiments, R⁴ is

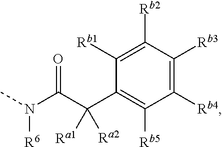

R³ is methyl, R² is H, and R¹ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl).

In certain embodiments, R¹ is C(═O)NH₂, R⁴ is

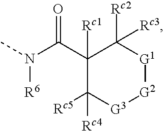

one or less of G¹, G², and G³ is a bond, and a pair of vicinal substituents selected from the group consisting of R^{c2}, R^{c3}, R^{c4}, R^{c5}, R^{c6}, R^{c7}, R^{c8}, R^{c9}, R^{c10}, and R^{c11} combine with the atoms to which they are bound to form a C₆-C₁₀ aryl.

In certain embodiments, R¹ is C(═O)NH₂, R⁴ is

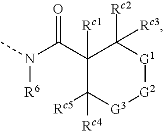

one or less of G¹, G², and G³ is a bond, and a pair of vicinal substituents selected from the group consisting of R^{c2}, R^{c3}, R^{c4}, R^{c5}, R^{c6}, R^{c7}, R^{c8}, R^{c9}, R^{c10}, and R¹¹ combine with the atoms to which they are bound to form a C₆-C₁₀ aryl.

In certain embodiments, Y is N, X is CR¹, one of R¹ and R⁴ is

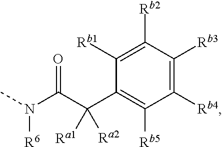

and at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy.

In certain embodiments, X is N, Y is CR², R⁴ is

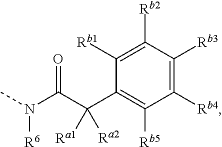

and at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy.

In certain embodiments, X is N, Y is N, R⁴ is

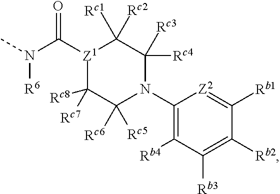

and Z¹ is CR^{c9}.

In certain embodiments, X is N, Y is N, R⁴ is

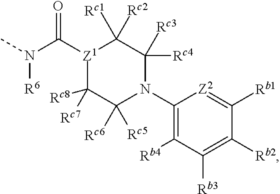

and Z² is CR^{b5}, and at least one selected from the group consisting of R^{b1}, R^{b2}, R^{b3}, R^{b4}, and R^{b5} is selected from the group consisting of halogen, C₁-C₆ alkyl, NO₂, and CN.

In certain embodiments, R¹ is H. In certain embodiments, R¹ is Me. In certain embodiments, R¹ is C(═O)NH₂. In certain embodiments, R¹ is C(═O)NMe₂. In certain embodiments, R¹ is C(═O)NEt₂. In certain embodiments, R¹ is C(═O)OEt. In certain embodiments, R¹ is C(═O)OMe. In certain embodiments, R¹ is C(═O)OEt. In certain embodiments, R¹ is C(═O)Ot-Bu. In certain embodiments, R¹ is C(═O)O(CH₂)₂NH₂. In certain embodiments, R¹ is C(═O)NH(CH₂)₃NH₂. In certain embodiments, R¹ is

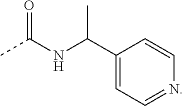

In certain embodiments, R¹ is

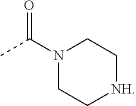

In certain embodiments, R¹ is

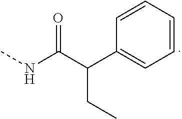

In certain embodiments, R¹ is

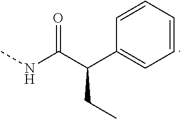

In certain embodiments, R¹ is

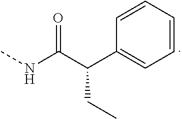

In certain embodiments, R¹ is

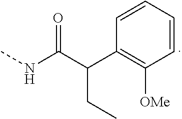

In certain embodiments, R¹ is

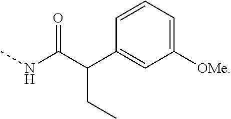

In certain embodiments, R¹ is

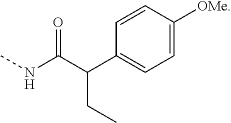

In certain embodiments, R¹ is

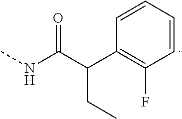

In certain embodiments, R¹ is

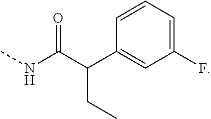

In certain embodiments, R¹ is

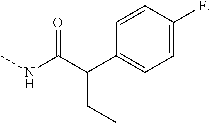

In certain embodiments, R¹ is

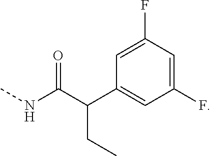

In certain embodiments, R¹ is

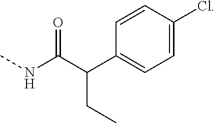

In certain embodiments, R¹ is

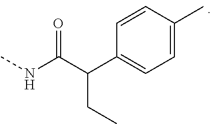

In certain embodiments, R¹ is

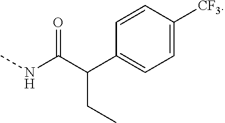

In certain embodiments, R¹ is

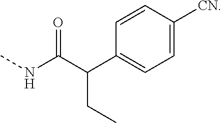

In certain embodiments, R¹ is

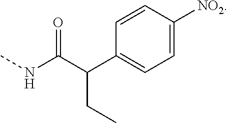

In certain embodiments, R¹ is

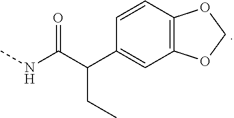

In certain
embodiments, R¹ is

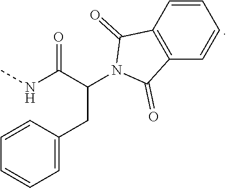

In certain embodiments, R¹ is

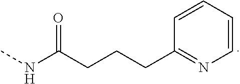

In certain embodiments, R¹ is

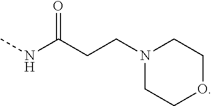

In certain embodiments, R¹ is

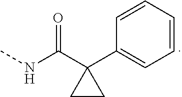

In certain embodiments, R¹ is

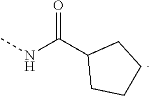

In certain embodiments, R¹ is

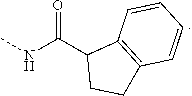

In certain embodiments, R¹ is

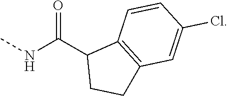

In certain embodiments, R¹ is

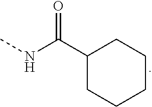

In certain embodiments, R¹ is

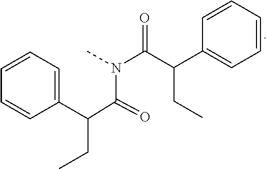

In certain embodiments, R¹ is

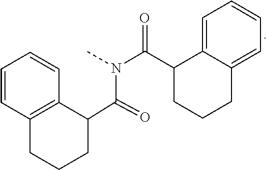

In certain embodiments, R¹ is

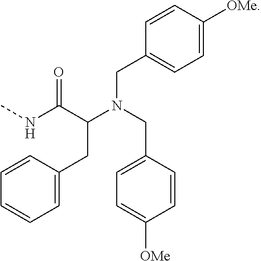

In certain embodiments, R¹ is

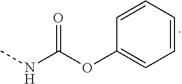

In certain embodiments, R¹ is

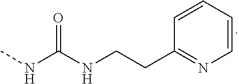

In certain embodiments, R¹ is

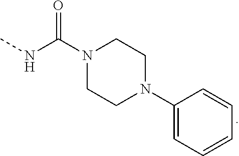

In certain embodiments, R¹ is

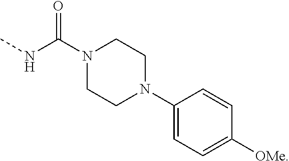

In certain embodiments, R¹ is

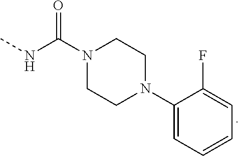

In certain embodiments, R¹ is

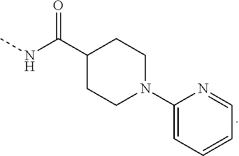

In certain embodiments, R¹ is

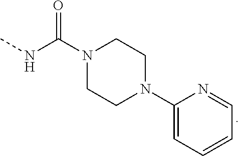

In certain embodiments, R⁴ is Me. In certain embodiments, R⁴ is C(═O)NH₂. In certain embodiments, R⁴ is C(═O)NMe₂. In certain embodiments, R⁴ is C(═O)NEt₂. In certain embodiments, R⁴ is C(═O)OEt. In certain embodiments, R⁴ is C(═O)OMe. In certain embodiments, R⁴ is C(═O)OEt. In certain embodiments, R⁴ is C(═O)Ot-Bu. In certain embodiments, R⁴ is C(═O)O(CH₂)₂NH₂. In certain embodiments, R⁴ is C(═O)NH(CH₂)₃NH₂. In certain embodiments, R⁴ is

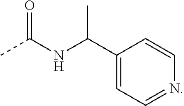

In certain embodiments, R⁴ is

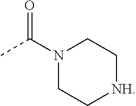

In certain embodiments, R⁴ is

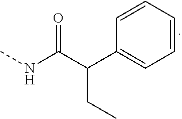

In certain embodiments, R⁴ is

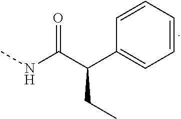

In certain embodiments, R⁴ is

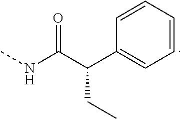

In certain embodiments, R⁴ is

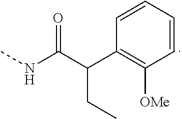

In certain embodiments, R⁴ is

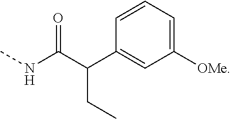

In certain embodiments, R⁴ is

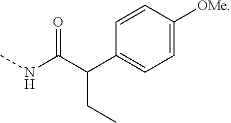

In certain embodiments, R⁴ is

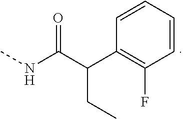

In certain embodiments, R⁴ is

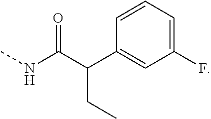

In certain embodiments, R⁴ is

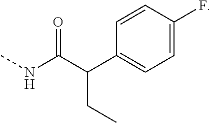

In certain embodiments, R⁴ is

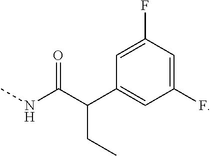

In certain embodiments, R⁴ is

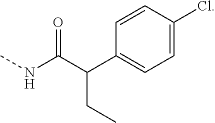

In certain embodiments, R⁴ is

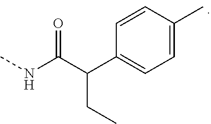

In certain embodiments, R⁴ is

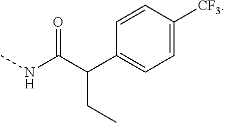

In certain embodiments, R⁴ is

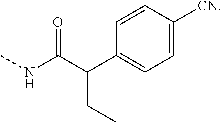

In certain embodiments, R⁴ is

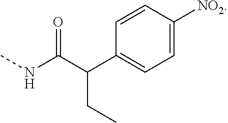

In certain embodiments, R⁴ is

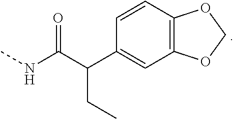

In certain embodiments, R⁴ is

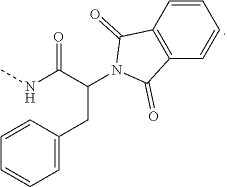

In certain embodiments, R⁴ is

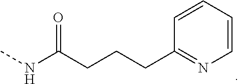

In certain embodiments, R⁴ is

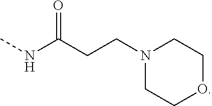

In certain embodiments, R⁴ is

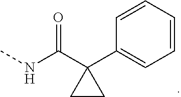

In certain embodiments, R⁴ is

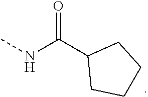

In certain embodiments, R⁴ is

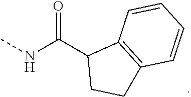

In certain embodiments, R⁴ is

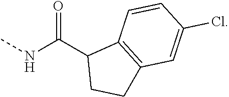

In certain embodiments, R⁴ is

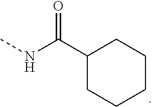

In certain embodiments R⁴ is

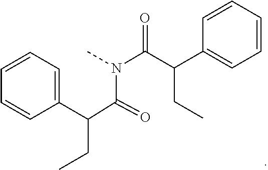

In certain embodiments, R⁴ is

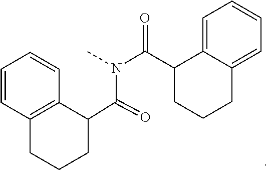

In certain embodiments, R⁴ is

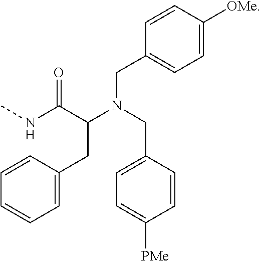

In certain embodiments, R⁴ is

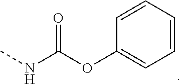

In certain embodiments, R⁴ is

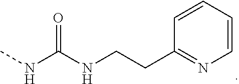

In certain embodiments, R⁴ is

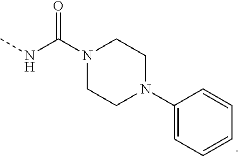

In certain embodiments, R⁴ is

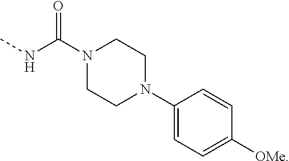

In certain embodiments, R⁴ is

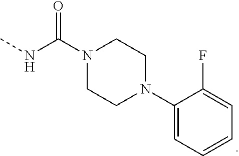

In certain embodiments, R⁴ is

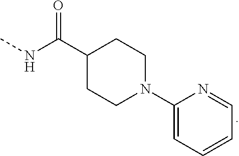

In certain embodiments, R⁴ is

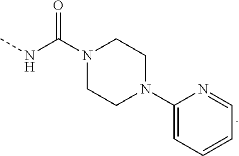

In certain embodiments, R² is H. In certain embodiments, R² is Me. In certain embodiments, R² is Et.

In certain embodiments, R³ is H. In certain embodiments, R³ is Me. In certain embodiments, R³ is Et. In certain embodiments, R³ is C(═O)OMe. In certain embodiments, R³ is C(═O)OEt. In certain embodiments, R³ is C(═O)NH₂. In certain embodiments, R³ is CN. In certain embodiments, R³ is Ph. In certain embodiments, R³ is 4-trifluoromethylphenyl. In certain embodiments, R³ is 4-fluorophenyl. In certain embodiments, R³ is

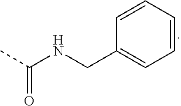

In certain embodiments, R³ is

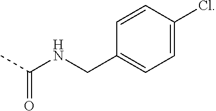

In certain embodiments R³ is

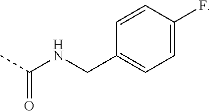

In certain embodiments, R³ is

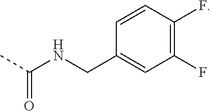

In certain embodiments, R³ is

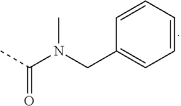

In certain embodiments, R³ is

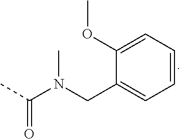

In certain embodiments, R³ is

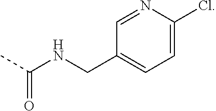

In certain other embodiments, the compound is not selected from the group consisting of 1, 2, 4, 5, 8, 9, 15, 16, 17, 19, 24, 26, 29, 31, 40, 66, 72, 76, 77, 78, 83, 84, and 86.

In certain other embodiments, the compound is selected from the group consisting of 1, 2, 4, 5, 8, 9, 15, 16, 17, 19, 24, 26, 31, 40, 66, 72, 76, 77, 78, 83, 84, and 86, 99, and 111.

In certain other embodiments, the compound is selected from the group consisting of 22, 29, 32, 33, 34, 35, 36, 39, 47, 50, 57, 68, 75, 82, 87, 89, 90, 95, 96, 97, 98, 99, 106, 109, 110, 111, 112, 114, 115, 116, 118, 119, 123, 124, 127, 130, 131, 132, 134, 135, 144, 153, 154, 155, 159, 160, 161, 162, 163, 165, 167, 168, 169, 170, 172, 173, 174, 175, 179, 180, 181, 182, 183, 184, 185, 186, 187, 188, 189, 190, and 191.

The compounds described herein can form salts with acids and/or bases, and such salts are included in the present invention. In certain other embodiments, the salts are pharmaceutically acceptable salts. The term “salts” embraces addition salts of free acids and/or bases that are useful within the methods of the invention. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hemisulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, ammonium, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound. Salts may be comprised of a fraction of less than one, one, or more than one molar equivalent of acid or base with respect to any compound of the invention. In certain other embodiments, the at least one compound of the invention is a component of a pharmaceutical composition further including at least one pharmaceutically acceptable carrier.

The compounds of the invention may possess one or more stereocenters, and each stereocenter may exist independently in either the (R) or (S) configuration. In certain other embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain other embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain other embodiments, the compounds described herein exist in solvated forms with pharmaceutically acceptable solvents such as water, and ethanol. In other embodiments, the compounds described herein exist in unsolvated form.

In certain other embodiments, the compounds of the invention exist as tautomers. All tautomers are included within the scope of the compounds recited herein.

In certain other embodiments, compounds described herein are prepared as prodrugs. A “prodrug” is an agent converted into the parent drug in vivo. In certain other embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain other embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain other embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain other embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain other embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and in the art. General methods for the preparation of compound as described herein are modified by the use of appropriate reagents and conditions, for the introduction of the various moieties found in the formula as provided herein.

### Pharmaceutical Compositions

In one aspect, the present disclosure provides a pharmaceutical composition comprising at least one compound of the invention and at least one pharmaceutically acceptable carrier and/or excipient.

In one aspect, the present disclosure provides a pharmaceutical composition comprising at least one compound selected from the group consisting of compounds 1, 2, 4, 5, 8, 9, 15, 16, 17, 19, 24, 26, 29, 31, 40, 66, 72, 76, 77, 78, 83, 84, 86, 99, and 111 and at least one pharmaceutically acceptable excipient.

In certain embodiments, the at least one compound is compound 111.

In certain embodiments, the at least one pharmaceutically acceptable excipient is at least one selected from the group consisting of water, polyethylene glycol (PEG) 400, PEG 300, propylene glycol (PG), benzyl alcohol, polysorbate 80, diethylene glycol monoethyl ether (DEGEE), isopropyl myristate, ethanol, diisopropyl adipate, C₁₂₋₁₅ alkyl lactate, thickening agent, hydroxypropyl cellulose, and PEG 4000.

In certain embodiments, the thickening agent comprises concentrated dispersion of acrylamide and sodium acryloyldimethyl taurate copolymer in isohexadecane. In certain embodiments, the thickening agent is SEPINEO™ P600.

In certain embodiments, the pharmaceutical composition comprises at least one of:
(a) compound 111, which comprises about 0.1% to about 10.0% (w/w) of the pharmaceutical composition;
(b) water, which comprises about 10% to about 15% (w/w) of the pharmaceutical composition;
(c) PEG 400, which comprises about 20% to about 40% (w/w) of the pharmaceutical composition;
(d) PEG 300, which comprises about 35% to about 60% (w/w) of the pharmaceutical composition;
(e) PG, which comprises about 5% to about 15% (w/w) of the pharmaceutical composition;
(f) benzyl alcohol, which comprises about 0.1% to about 5% (w/w) of the pharmaceutical composition;
(g) polysorbate 80, which comprises about 1% to about 10% (w/w) of the pharmaceutical composition;
(h) DEGEE, which comprises about 1% to about 15% (w/w) of the pharmaceutical composition;
(i) isopropyl myristate, which comprises about 0.1% to about 5% (w/w) of the pharmaceutical composition;
(j) ethanol, which comprises about 5% to about 15% (w/w) of the pharmaceutical composition;
(k) diisopropyl adipate, which comprises about 5% to about 15% (w/w) of the pharmaceutical composition;
(l) C₁₂₋₁₅ alkyl lactate, which comprises about 5% to about 15% (w/w) of the pharmaceutical composition
(m) thickening agent, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

In certain embodiments, the pharmaceutical composition comprises, consists of, or consists essentially of 111 (about 1.0% w/w), PEG 400 (about 24.3% w/w), PEG 300 (about 40.0% w/w), PG (about 10.0% w/w), benzyl alcohol (about 2.7% w/w), polysorbate 80 (about 5.0% w/w), DEGEE (about 5.0% w/w), isopropyl myristate (about 2.0% w/w), and PEG 4000 (about 10% w/w).

In certain embodiments, the pharmaceutical composition comprises, consists of, or consists essentially of 111 (about 1.0% w/w), PEG 400 (about 23.6% w/w), PEG 300 (about 40.0% w/w), PG (about 10.0% w/w), benzyl alcohol (about 2.7% w/w), polysorbate 80 (about 5.0% w/w), DEGEE (about 5.0% w/w), and PEG 4000 (about 10.0% w/w).

In certain embodiments, the pharmaceutical composition comprises, consists of, or consists essentially of 111 (about 1.0% w/w), PEG 400 (about 34.0% w/w), PEG 300 (about 40.0% w/w), PG (about 10% w/w), benzyl alcohol (about 2.0% w/w), polysorbate 80 (about 5.0% w/w), DEGEE (about 5.0% w/w), isopropyl myristate (about 2.0% w/w), and hydroxypropyl cellulose (about 1.0% w/w).

In certain embodiments, the pharmaceutical composition comprises, consists of, or consists essentially of 111 (about 1.0% w/w), water (about 13.3% w/w), PEG 300 (about 57.0% w/w), PG (about 10% w/w), benzyl alcohol (about 2.7% w/w), polysorbate 80 (about 5.0% w/w) DEGEE (about 5.0% w/w), isopropyl myristate (about 2.0% w/w), and thickening agent (about 4% w/w).

In certain embodiments, the pharmaceutical composition comprises, consists of, or consists essentially of 111 (about 1.0% w/w), PEG 300 (about 48.0% w/w), PG (about 10% w/w), benzyl alcohol (about 1.5% w/w), DEGEE (about 10% w/w), ethanol (about 8.5% w/w), diisopropyl adipate (about 10% w/w), C₁₂₋₁₅ alkyl lactate (about 10% w/w), and hydroxypropyl cellulose (about 1.0% w/w).

In certain embodiments, the at least one compound is compound 99.

In certain embodiments, the at least one pharmaceutically acceptable excipient is at least one selected from the group consisting of water, PEG 400, PG, benzyl alcohol, polysorbate 80, DEGEE, isopropyl myristate, ethanol, diisopropyl adipate, C₁₂₋₁₅ alkyl lactate, dimethyl isosorbide, PEG 40 hydrogenated castor oil (HCO), hydroxypropyl cellulose, and PEG 4000.

In certain embodiments, the pharmaceutical composition comprises at least one of:
(a) compound 99, which comprises about 0.1% to about 10.0% (w/w) of the pharmaceutical composition;
(b) water, which comprises about 1% to about 10% (w/w) of the pharmaceutical composition;
(c) PEG 400, which comprises about 25% to about 35% (w/w) of the pharmaceutical composition;
(d) PG, which comprises about 10% to about 30% (w/w) of the pharmaceutical composition;
(e) benzyl alcohol, which comprises about 0.1% to about 5% (w/w) of the pharmaceutical composition;
(f) polysorbate 80, which comprises about 1% to about 10% (w/w) of the pharmaceutical composition;
(g) DEGEE, which comprises about 10% to about 50% (w/w) of the pharmaceutical composition;
(h) isopropyl myristate, which comprises about 0.1% to about 5% (w/w) of the pharmaceutical composition;
(i) ethanol, which comprises about 20% to about 35% (w/w) of the pharmaceutical composition;
(j) diisopropyl adipate, which comprises about 5% to about 15% (w/w) of the pharmaceutical composition;
(k) C₁₂₋₁₅ alkyl lactate, which comprises about 1% to about 15% (w/w) of the pharmaceutical composition;
(l) dimethyl isosorbide, which comprises about 5% to about 15% (w/w) of the pharmaceutical composition;
(m) PEG 40 HCO, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

In certain embodiments, the pharmaceutical composition comprises, consists of, or consists essentially of 99 (about 1.0% w/w), PEG 400 (about 28.3% w/w), PG (about 20.0% w/w), benzyl alcohol (about 2.7% w/w), polysorbate 80 (about 5.0% w/w), DEGEE (about 25.0% w/w), isopropyl myristate (about 2.0% w/w), dimethyl isosorbide (about 10.0% w/w), PEG 40 HCO (about 5.0% w/w), and hydroxypropyl cellulose (about 1.0% w/w).

In certain embodiments, the pharmaceutical composition comprises, consists of, or consists essentially of 99 (about 1.0% w/w), PEG 400 (about 30.30% w/w), PG (about 20.0% w/w), benzyl alcohol (about 2.7% w/w), polysorbate (about 5.0% w/w), DEGEE (about 25.0% w/w), dimethyl isosorbide (about 10.0% w/w), PEG 40 HCO (about 5.0% w/w), and hydroxypropyl cellulose (about 1.0% w/w).

In certain embodiments, the pharmaceutical composition comprises, consists of, or consists essentially of 99 (about 1.0% w/w), PEG 400 (about 19.30% w/w), PG (about 20.0% w/w), benzyl alcohol (about 2.7% w/w), polysorbate (about 5.0% w/w), DEGEE (about 25.0% w/w), isopropyl myristate (about 2.0% w/w), dimethyl isosorbide (about 10.0% w/w), PEG 40 HCO (about 5.0% w/w), and PEG 4000 (about 10.0% w/w).

In certain embodiments, the pharmaceutical composition comprises, consists of, or consists essentially of 99 (about 1.0% w/w), DEGEE (about 40.0% w/w), ethanol (about 28.0% w/w), diisopropyl adipate (about 10.0% w/w), C₁₂₋₁₅ alkyl lactate (about 10.0% w/w), dimethyl isosorbide (about 10.0% w/w), and hydroxypropyl cellulose (about 1.0% w/w).

In certain embodiments, the pharmaceutical composition comprises, consists of, or consists essentially of 99 (about 1.0% w/w), water (about 5.0% w/w), DEGEE (about 42.5% w/w), ethanol (about 25.0% w/w), diisopropyl adipate (about 10.0% w/w), C₁₂₋₁₅ alkyl lactate (about 5.0% w/w), dimethyl isosorbide (about 10.0% w/w), and hydroxypropyl cellulose (about 1.5% w/w).

In certain embodiments, the pharmaceutical composition is formulated for topical administration. In certain embodiments, the topical formulation comprises a gel or ointment.

### Methods

The invention includes methods of treating, ameliorating, and/or preventing an orthopoxvirus infection in a human subject. In certain embodiments, the orthopoxvirus infection is caused by Molluscum contagiosum virus (MCV). In certain embodiments, the orthopoxvirus infection is caused by camelpox virus. In certain embodiments, the orthopoxvirus infection is caused by cowpox virus. In certain embodiments, the orthopoxvirus infection is caused by mousepox virus. In certain embodiments, the orthopoxvirus infection is caused by horsepox virus. In certain embodiments, the orthopoxvirus infection is caused by monkeypox virus. In certain embodiments, the orthopoxvirus infection is caused by raccoonpox virus. In certain embodiments, the orthopoxvirus infection is caused by tanapox virus. In certain embodiments, the orthopoxvirus infection is caused by varioloa (smallpox virus). In certain embodiments, the orthopoxvirus infection is caused by Yoka poxvirus. In certain embodiments, the orthopoxvirus infection is caused by cervidpoxvirus (deerpox). In certain embodiments, the orthopoxvirus infection is caused by avipoxvirus (fowlpox). In certain embodiments, the orthopoxvirus infection is caused by capripoxvirus (goatpox). In certain embodiments, the orthopoxvirus infection is caused by leporipoxvirus (myxoma virus). In certain embodiments, the orthopoxvirus infection is caused by parapoxvirus (orf virus). In certain embodiments, the orthopoxvirus infection is caused by suipoxvirus (swinepox). In certain embodiments, the orthopoxvirus infection is caused by vatapoxvirus (Yaba-like disease virus). In certain embodiments, the method comprises administering to the subject a therapeutically effective amount of a compound of the invention, or pharmaceutically acceptable salts, solvates, enantiomers, diastereoisomers, geometric isomers, or tautomers thereof.

In certain embodiments, the compound of the present invention is a compound of formula (II):

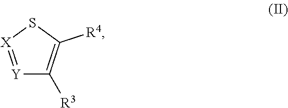

X is CR¹ or N;
Y is CR² or N;
R¹ is H, optionally substituted C₁-C₆ alkyl, —C(═O)NR⁶R⁶, —C(═O)OR⁶, or R⁸;
R² is H or optionally substituted C₁-C₆ alkyl;
R³ is H, —CN, —C(═O)OR⁶, —C(═O)NR⁶R⁶, optionally substituted phenyl, or optionally substituted C₁-C₆ alkyl;
R⁴ is —C(═O)OR⁶ or R⁸;
R⁵ is optionally substituted C₁-C₆ alkyl or optionally substituted phenyl; each occurrence of R⁶ is independently H or optionally substituted C₁-C₆ alkyl, or two R⁶ bound to the same N form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷, wherein R⁷ is optionally substituted C₁-C₆ alkyl, optionally substituted cycloalkyl, CH (optionally substituted heterocyclyl)(R⁵), CH(R⁵)(R⁵), or optionally substituted 4-7 membered heterocyclyl, or R⁶ and R⁷ bound to the same N form optionally substituted 4-7 membered heterocyclyl;
with the proviso that (I) comprises a single R⁸;

In certain embodiments, the compound, or any composition comprising the same, is applied to the skin of an infected human. In other embodiments, the compound, or any composition comprising the same, is applied to at least one MCV lesion on the skin of the infected human. In yet other embodiments, the compound is formulated as a topical pharmaceutical composition. In certain embodiments, the topical pharmaceutical composition comprises a gel or ointment. In yet other embodiments, the compound, or any composition comprising the same, is administered topically to the infected human.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain other embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating, ameliorating, and/or preventing diseases and disorders discussed herein) in the composition. In yet other embodiments, the compound of the invention is the only biologically active agent (i.e., capable of treating, ameliorating, and/or preventing diseases and disorders discussed herein) in therapeutically effective amounts in the composition.

In certain other embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 300 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therein between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain other embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include intravitreal, oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravitreal, intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intravitreal, intraperitoneal, intramuscular, intrasternal injection, and kidney dialytic infusion techniques.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically- or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Controlled Release Formulations and Drug Delivery Systems

In certain other embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. In certain embodiments, the compounds of the invention can be formulated for sustained release over a period of 3-12 months.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain other embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### General Experimental Details

Reactions were not carried out under an inert atmosphere unless specified and all solvents and commercial reagents were used as received.

### Cell Handling

BSC-1 and Vero cells were cultured in DMEM supplemented with 5% FBS, penicillin, and streptomycin (growth medium). Plaque reduction assays were performed in 48-well plates and cytotoxicity studies in 96-well plates.

### Compounds

All compounds were declared as >95% pure by the vendors or by analysis. Unless noted, compounds were prepared as 10 mM solutions in DMSO.

LC/MS data (ESI+) were determined with a Waters Alliance 2695 HPLC/MS (Waters Symmetry C18, 4.6×75 mm, 3.5 m) or (Phenomenex C18, 4.6×75 mm, 3.0 m) with a 2996 diode array detector from 210-400 nm; the solvent system is 5-95% MeCN in water (with 0.1% TFA) over nine minutes using a linear gradient, and retention times are in minutes. Mass spectrometry was performed on a Waters ZQ using electrospray in positive mode. Preparative reversed phase HPLC was performed on a Waters Sunfire column (19×50 mm, C18, 5 m) with a 10 min mobile phase gradient of 10% acetonitrile/water to 90% acetonitrile/water with 0.1% TFA as buffer using 214 and 254 nm as detection wavelengths. Injection and fraction collection were performed with a Gilson 215 liquid handling apparatus using Trilution LC software. ¹H NMR were recorded on Varian Oxford 300 MHz. Chemical shifts (δ) are expressed in ppm downfield from tetramethylsilane (TMS) unless otherwise noted.

### Plaque Reduction Assay, In Vitro Processive DNA Synthesis, and Cytotoxicity

Plaque reduction assay was performed on BSC-1 cells for VACV infection and Vero cells for HSV-1 infection. Assessment of processive DNA synthesis was by way of the ELISA-based Rapid Plate Assay. Cytotoxicity was examined by cell proliferation by seeding BSC-1 cells at ˜10-15% confluence in a 96-well plate and treating with 2-fold serially diluted compound solutions for 3 d. DMSO was held at 1% throughout. Cell viability was measured by the CyQUANT GR dye according to the manufacture's recommendation (Invitrogen, USA). For 24 h-treatment, BSC-1 cells were seeded at ˜80% confluence and monitored for ATP production. Cells were removed of growth media and lysed with the addition of 100 μL of 1% Triton X-100 in PBS per well by incubating at room temperature for 10 min. Five microliter of the lysate was used for the luciferase/luminescence assay according to the manufacture's recommendation (Invitrogen, USA).

### Protein Expression, Purification, and Handling

Proteins were recombinantly expressed in Rosetta2pLysS, and purified by Ni-NTA resins and gel filtration column chromatography. Unless noted, D4 proteins were removed of the N-terminal His-tag by TEV protease and maintained in the eluting column buffer: 20 mM sodium phosphate, pH 6.8, 0.2 M NaCl, and 15% w/v glycerol.

### Differential Scanning Fluorimetry

A typical experiment combined compound with 0.5 μM of D4 in column buffer containing 2.5 mM DTT, 1% DMSO, and 0.005% Tween-20 (reaction buffer) in a 100-μL reaction volume. After 20-min incubation at 25° C., 4 μL of 25× Sypro Orange (diluted in the same buffer, yielding a final concentration of ˜0.96×) was added to the reaction mix and centrifuged at 15,000 rpm for 1 min to remove particulates. Twenty microliter of the supernatant was used for measurement and analysis.

### Drug Affinity Responsive Target Stability (DARTS)

DARTS experiments were adapted from Lomenick et al. (Lomenick, et al., 2009, PNAS USA 106(51):21984-21989). Bacterial cells expressing either N-terminal His (D4 and MBP-His₈) or MBP tag were induced with 0.1 mM IPTG overnight at room temperature, and 3-5 mL of cell suspension was pelleted and used for an experiment. Cells pellets were resuspended in 500 μL of 20 mM sodium phosphate, pH 6.8, 0.2 M NaCl, and 0.5% Triton X-100 and lysed by sonication. After 5-min centrifugation at 15,000 rpm, the cleared lysate was diluted 40-fold into reaction buffer, giving an effective Triton X-100 concentration 0.012%. A typical reaction mix was carried out in 100 μL volume. Proteolysis was achieved with the addition of 5 μL Pronase (Calbiochem, USA; prepared as 10 mg/mL stock in 50 mM Tris, pH 8, and 40 mM Ca²⁺) and incubation at 30° C. for 30 min. For compound binding, the diluted lysates were combined with 2-fold serial dilutions of compounds (prepared as 10 mM stocks in DMSO) and incubated for 1 h at 25° C. Reactions were stopped with the addition of 20 μL of 5×SDS-PAGE loading buffer and heated at 90° C. for 5 min. Fifteen microliter of the mix was loaded onto a 4-12% Bis-Tris gel, resolved with MOPS buffer, and transferred onto a PVDF membrane at 20 V for 2 h. Western blotting was accomplished with 1:500 anti-His (GE Healthcare, USA) or 1:2000 anti-MBP (New England Biolabs, USA) antibody.

### Maleimide Dye Conjugation

Cysteine substitutions were introduced at positions 219 (Δ219C), and −19 (−19C; 19-aa upstream of Met of D4 and immediately after the start codon) to permit conjugation to fluorescein-5-maleimide (AnaSpec Inc., USA) and N-(1-pyrene)maleimide (ThermoFisher Scientific, USA). Dyes were prepared as 10 mg/mL stocks in DMSO. During the gel filtration step of protein purification, proteins were treated with 10 mM DTT for 30 min at room temperature prior to loading onto the column. Protein eluates were maintained at <25 μM concentrations and immediately added with four molar equivalents of dyes in column buffer containing 0.01% Triton X-100 and 5 mM EDTA. The reactions were left overnight at 4° C. away from light. The protein/dye mixes were then centrifuged to remove particulates and the supernatants passed twice through Bio-Beads SM2 resins (Bio-Rad, USA) to remove the Triton X-100. The eluates were further concentrated and purified by gel filtration to remove unbound dyes and EDTA.

### Isothermal Titration Calorimetry

Fish sperm DNA (USB Scientific, USA) was dissolved in water and fragmented by sonication in order to generate a random DNA mix. The fragmented DNA was then dialyzed into column buffer absence of glycerol. The 15-mer oligomer comprised of poly d(TC) (Integrated DNA Technologies, USA) was directly prepared in the same buffer without the dialysis step. DNA and compounds were prepared in the same dialysis buffer with added 2.5 mM DTT, 1% DMSO, and 0.005% Tween-20. DNA binding was then assessed by isothermal titration calorimetry (ITC) on a MicroCal iTC200 microcalorimeter (Malvern Instruments, United Kingdom) by titrating 3 μL/injection of 1.2 mg/mL of random DNA (with unknown molar concentration) or 5.3 mg/L of single-stranded DNA into the sample consisting of 40 μM of compound. Experiments were performed at 25° C. with 800 rpm stirring and 3-min spacing in order to allow equilibration. Ethidium bromide (Sigma-Aldrich, USA) was freshly prepared in water. Since the molar concentrations of the DNA ligands were undefined, only raw heat values are depicted.

### Steady-State Fluorescence

Protein fluorescence was carried out on a PTI photon counter equipped with a Model 810 detection system (HORIBA Scientific, USA). D4 was prepared at 0.5 μM in column buffer added with 2.5 mM DTT, and 0.005% Tween-20 and measured in a quartz cuvette. Tryptophan emission was monitored at 329 nm after 295 nm excitation. For the examination of the effect of DMSO on tryptophan, DMSO was added last, mixed, and the emission recorded for 30 min at 1 data point/s. All spectra were buffer-subtracted to remove background fluorescence and scattering.

### Surface Plasmon Resonance

Experiments were performed on a Biacore X100 (GE Healthcare Life Sciences, USA) using filtered and degassed buffers at 25° C. Both flow cells of an NTA biosensor chip were charged with 0.5 mM Ni²⁺ for 120 s at 5 μL/min and activated for 7 min with a 1:1 mixture of 391 and 100 mM carbodiimide hydrochloride and N-hydroxysuccinimide, respectively, in 10 mM HEPES (pH 8), 200 mM NaCl, and 0.005% Tween-20. His-tagged MBP and D4 proteins were prepared in the same buffer containing 15% glycerol at 0.01 mg/mL concentrations, and injected onto the reference and active cells, respectively, for >30 min in order to achieve maximum crosslinked proteins. Approximately 1000 and 3000 response unit (RU) of MBP and D4, respectively, were obtained after quenching with 0.5 M ethanolamine for 15 min. For compound-binding studies, the running buffer was 10 mM HEPES (pH 6.8), 200 mM NaCl, 0.5 mg/mL carboxymethyl dextran, 1% DMSO, 0.01% sodium azide, and 0.005% Tween-20. Compounds were dissolved in running buffer, sonicated for 10 min, and centrifuged at 15000 rpm for 5 min. A typical experiment consisted of the injection of compound for 60 s at 10 μL/min and monitored for 180 s, followed by the same procedure for 1% DMSO injection alone. Data were analyzed by the included BIA evaluation software (GE Healthcare Life Sciences, USA). Each 1% DMSO injection was used for double-referencing for the corresponding compound injection, and the equilibrium response (R_{eq}) values were obtained by linear regression at steady-state, whereby negative equilibrium values were not used for fitting. Experiments were repeated twice.

### Processive DNA Synthesis Assays

Processive DNA synthesis was assessed using the Rapid Plate Assay (Lin & Ricciardi, 2000, J. Virol. Methods 88:219-225). A 5′-biotinylated 100-nucleotide template that contains adenines only at its 5′ distal end was annealed with a 15-nucleotide primer to its 3′ end and attached to streptavidin-coated 96-plate wells. DNA synthesis was carried out in 50 μL reaction mixture containing 100 mM (NH)₂SO₄, 20 mM Tris-HCl (pH 7.5), 3 mM MgCl₂, 0.1 mM EDTA, 0.5 mM DTT, 2% glycerol, 40 μg/ml BSA, 5 μM dATP, 5 μM dCTP, 5 μM dGTP, 1 μM digoxigenin-11-dUTP, and E9/A20/D4 proteins. The TNT reticulocyte lysate or in vitro translated luciferase was used as a negative control. After incubation at 37° C. for 30 min, the plate was washed extensively with phosphate-buffered saline (PBS). The wells were then incubated with anti-digoxigenin-peroxidase antibody for 1 h at 37° C., followed by washing with PBS. The substrate 2,2′-azino-bis(3-ethylbenzthiazoline)-sulfonate was added, and plates were gently rocked to allow color development. DNA synthesis was quantified by measuring the absorbance of each reaction at 405 nm with a microplate reader. Experiments were conducted in triplicate and independently repeated at least twice.

### Thermal Shift Assay

Thermal shift (differential scanning fluorimetry) assay was performed as previously described (Nuth, et al., 2011, J. Med. Chem. 54:3260-3267). Briefly, 5 μM purified 6His-mD4 was mixed with compounds in thin-wall PCR 96-plate wells at 20 μL total volume containing 25 mM phosphate buffer (pH 6.8), 0.2 M NaCl, 2.5% glycerol, 2% DMSO, 0.005% (w/v) Triton-X100, and 1× Sypro Orange. Fluorescence intensities were monitored using the Applied Biosystems 7500 Fast Real-Time PCR system at 582 nm from 25-80° C. at a rate of 1° C./min. To generate melting temperature (Tₘ), protein melting curves were plotted on GraphPad Prism and fitted to the Boltzmann sigmoidal model. Thermal shift (ΔTₘ) is the difference between the 2% DMSO mock-treatment and inhibitor treatment. All experiments were duplicated and repeated independently.

### Viral Plaque Reduction and Cytotoxicity Assays

Viral plaque reduction assay was performed using BSC-1 cells (Nuth, et al., 2011, J. Med. Chem. 54:3260-3267) in triplicate. Briefly, cells were infected by adsorbing virus at 80 PFU/well in 100 μL of growth medium for 1 h in 48-well plate, followed by 16 h treatment with compounds. Cells were stained and plaques counted under dissecting microscope and data was plotted on GraphPad Prism.

### Franz Cell Apparatus

The Franz cell apparatus and receptor medium is equilibrated to 35° C.±1° C. Dermatomed human cadaver torso skin is thawed and its integrity is checked by measuring the trans-epidermal water loss with a Vapometer. Following assembly of the apparatus, the skin and media are equilibrated for 30 mins. Temperature of the skin is verified with an infrared laser thermometer and a pre-dose (T=0) sample is taken from the receptor medium. Mixed formulations (10 mg API/mL) are applied to the skin surface (0.01 mL over an area of 1 cm²) and dosing time is recorded. Next, 0.5 mL samples of receptor medium are taken at 2, 4, 6, 21 and 24 h post-dose and sample volumes are replenished with fresh warm medium. After the final timepoint, the surface of the skin is washed with 0.5 mL PBS to remove residual formulation and the surface is blotted dry with a cotton swab. The washing procedure is repeated twice. The skin is removed from the apparatus, spread on a flat surface and the stratum corneum is removed by tape stripping (typically 4 strips). The stratum corneum is recovered by rinsing the tape strips. The stripped skin is then placed on a heat block (pre-warmed to 60° C.) for 1-2 min to heat separate the skin layers. The epidermis is peeled away using forceps and the epidermal and dermal skin layers are weighed and homogenized in 1×PBS/4% BSA. Protein-extracted samples are stored at −20° C. until further analysis by LC-MS/MS.

### Example 1: Protein Dynamics

Previous thermodynamic results suggested that D4 contains protein flexibility that contributed to overall protein dynamics. This protein dynamics was speculated to be necessary for protein function, as well as being responsible for promoting the observed protein sizing heterogeneity in vitro. Therefore, compounds that can disrupt the dynamics can be used as design leads. As such, it was necessary to establish D4 was indeed exhibiting dynamic properties.

As an initial approach, effects of DMSO, the preferred organic solvent for compound preparation, on overall intrinsic tryptophan fluorescence were investigated. Since tryptophan emission is sensitive to solvent exposure, such an approach can provide insights into the environment surrounding the five intrinsic tryptophan residues. Moreover, it was also important to ensure that DMSO was not adversely affecting protein structure, since the addition of ethanol was shown to readily promote protein aggregation, thus raising the concern that compound-binding experiments using DMSO could be hampered by the protein heterogeneity.

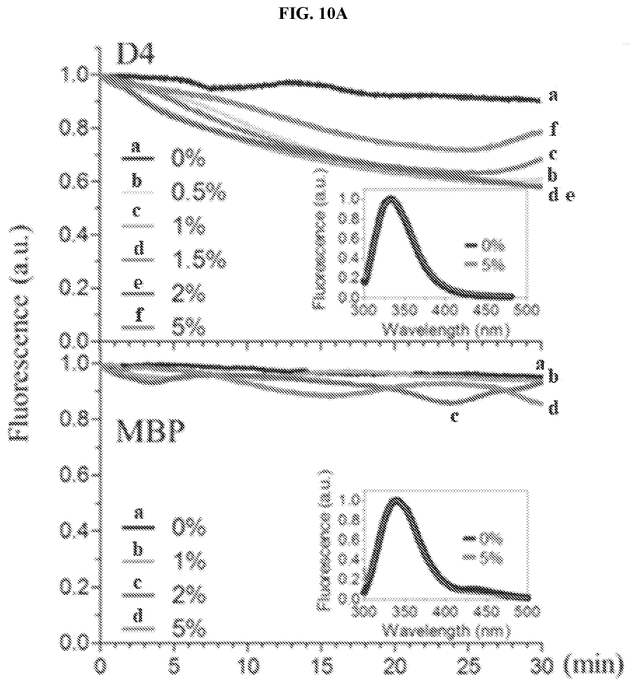

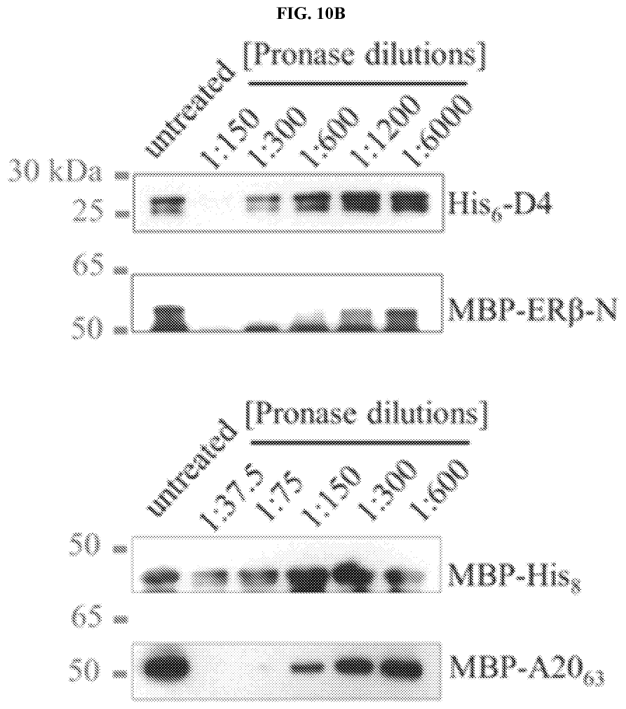

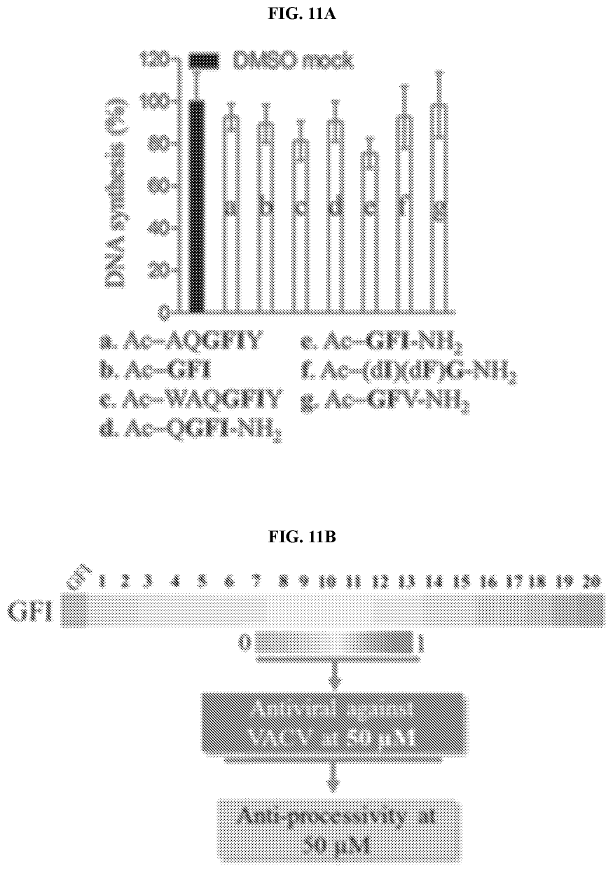

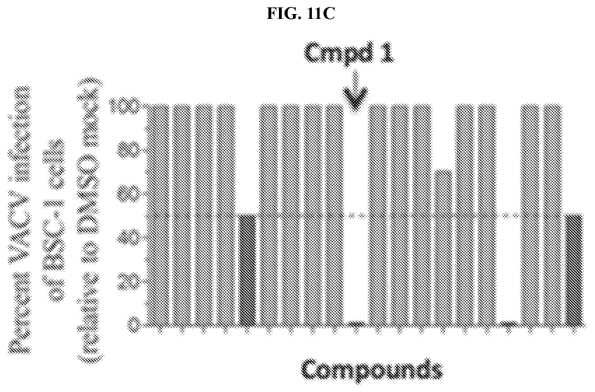

Tryptophan emission was monitored over a 30-min period in the presence of increasing DMSO concentrations, a time-frame typical for compound incubation with protein. As shown in FIG. 10A, significant decrease in emission was observed for 0.5-5% DMSO. By contrast, the well-folded maltose binding protein (MBP) lacked a similar trend. Given that tryptophan quenching is mediated through a solvent-stabilized charge-transfer of the ring-to-peptide backbone, this suggests that the observed fluctuation in fluorescence likely arose from the DMSO-H₂O exchange at the protein surface, which can conceivably be accelerated by a flexible protein. Examination of the fluorescence spectra for both D4 and MBP at 5% DMSO in comparison to 0% DMSO showed no evidence of spectral shifts, thus ruling out disruption of protein fold by DMSO (FIG. 10A, insets).

In certain embodiments, a protein with exhibited flexibility/dynamics is more prone to digestion by a protease due to the transient exposure of buried sites. In order to test this hypothesis, bacterial lysates expressing proteins of interest were exposed to varying concentrations of the nonspecific protease, Pronase, according to the drug affinity responsive target stability (DARTS) set-up and probed by Western blotting. Specifically, the protein examples were chosen on the basis of their relative protein folds. As shown in FIG. 10B, D4 was largely undetected at the tested 1:150 Pronase dilution (which corresponded to 3.2 μg/mL of protease in the reaction mix). By comparison, the well-folded MBP showed reasonable protection to proteolysis even at 1:37.5 Pronase dilution (FIG. 10B, corresponding to 12.7 μg/mL of protease). The addition of the N-terminal 63-amino acid portion of A20 onto MBP subsequently rendered the protein more susceptible to proteolysis under the same Pronase dilutions (FIG. 10B), lending credence to the suggested disorder/unfolded state of A20₆₃. In order to compare the susceptibility of a protein with known flexibility/dynamics to Pronase digestion, a MBP fusion protein of the N-terminal 103-aa of human estrogen receptor beta (ERβ-N) was constructed; in the absence of a carrier protein, it was expressed as an inclusion body that can be refolded in vitro into an intrinsically disordered structure. As an MBP fusion, ERβ-N was detected as a minor product in the crude cell lysate and showed strong susceptibility to protease digestion even at 1:1200 Pronase dilution (FIG. 10B). Taken together, the solvent exposure and DARTS results provided consistent demonstration of protein dynamics and showed D4 neither exhibited properties equivalent to a well-folded protein such as MBP nor that with defined disorder.

### Example 2: Compound 1 Inhibits Poxvirus DNA Synthesis

In vitro processive DNA synthesis was assessed by combining DNA polymerase with processivity factor in an ELISA-based assay. For the examination of VACV, proteins of DNA polymerase, A20, and D4 were combined, while UL30 (DNA polymerase) and UL42 (processivity factor) were combined for HSV-1. In accordance with the lack of antiviral activity against HSV-1 infection by Compound 1 (FIG. 8B), no inhibition of DNA synthesis was observed using HSV-1 proteins (FIG. 8A). By comparison, Compound 1 was capable of inhibiting DNA synthesis using poxvirus proteins. Specifically, when D4 of molluscum contagiosum virus (MCV) was substituted into the VACV reaction (i.e., DNA polymerase and A20, absent of VACV D4), inhibition of DNA synthesis was observed (IC₅₀=13.4 μM) (FIG. 6). Nearly identical inhibition was observed for VACV.

### Example 3: D4 is a Target of Compound 1

In order to investigate D4 as the intended target of compound 1, protein binding was initially examined by incubating compound with purified D4 proteins and measured by differential scanning fluorimetry (DSF). With increasing compound, a dose-dependent decrease in thermal shift was observed (ΔTₘ=−0.86 and −1.55 for 25 and 50 μM treatments, respectively), with the fluorescence signals for all curves for up to 50 μM compound treatment showing comparable maxima, indicating minimal assay interference (e.g., protein precipitation) by the compound (FIG. 12A, Table 1). By comparison, the drugs cidofovir (CDV) and tecovirimat (also known as ST-246), both of which shown to be effective against various poxviruses, displayed no thermal shifts (within experimental errors) for up to 50 μM (Table 1).

Next, compound-binding was examined by surface plasmon resonance (SPR). Using a senor chip NTA, his-tagged D4 was captured onto the Ni-charged active flow cell and crosslinked, while his-tagged MBP was similarly prepared for the reference flow cell to serve as a matching and unrelated protein surface. As shown in FIG. 12B, a dose-response was observed for compound 1 binding to D4, yielding a binding affinity K_{D}=22.8 μM as estimated by steady-state analysis. By comparison, near-baseline signals for up to 50 μM compound and a lack of dose-response were observed for both CDV and ST-246, with CDV showing slight binding to the active flow cell at 25 and 50 μM concentrations (FIG. 12B). In order to ensure the specificity of compound 1 for D4, compound-binding was further investigated by DARTS by incubating test compounds with crude cell lysates expressing D4 or control proteins. Given that the extent of protein folding dictated the efficiency of the proteolysis (FIG. 10B), Pronase was variably added as to permit a discernible difference (typically >50%) in proteolysis between the untreated and mock treatments (FIGS. 12C-12D). Since the binding of a compound impedes the proteolytic degradation of the intended target, the observed increase in D4 protein levels, in comparison to the mock treatment, with increasing concentrations of compound 1 was consistent with D4 as the target (FIG. 12C). By contrast, CDV and ST-246 did not affect the protein levels of D4 at concentrations up to 100 μM (FIG. 12D). Similarly, the inability of compound 1 to impede the levels of the unrelated proteins MBP and ERβ-N further provided evidence of specificity for D4 (FIG. 12C).

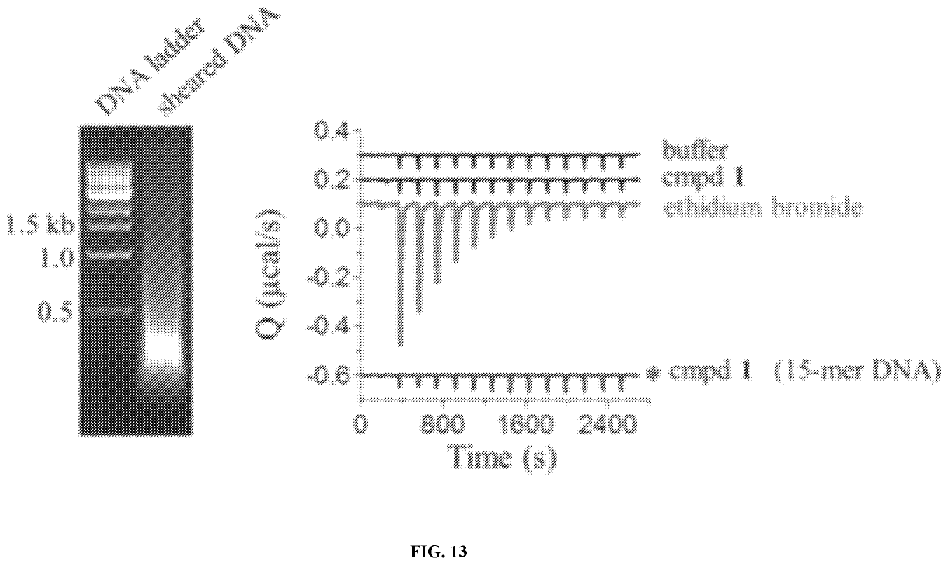

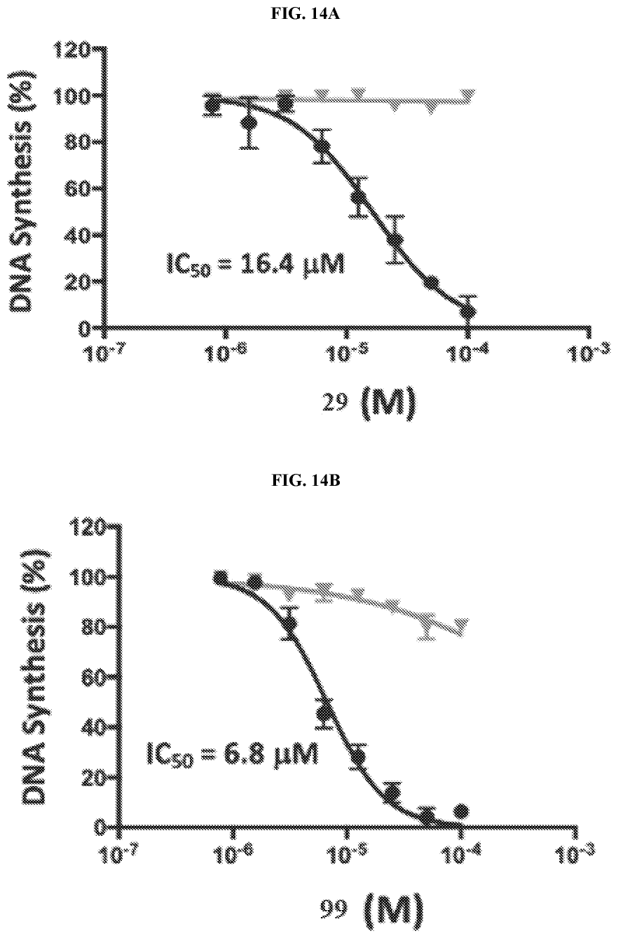

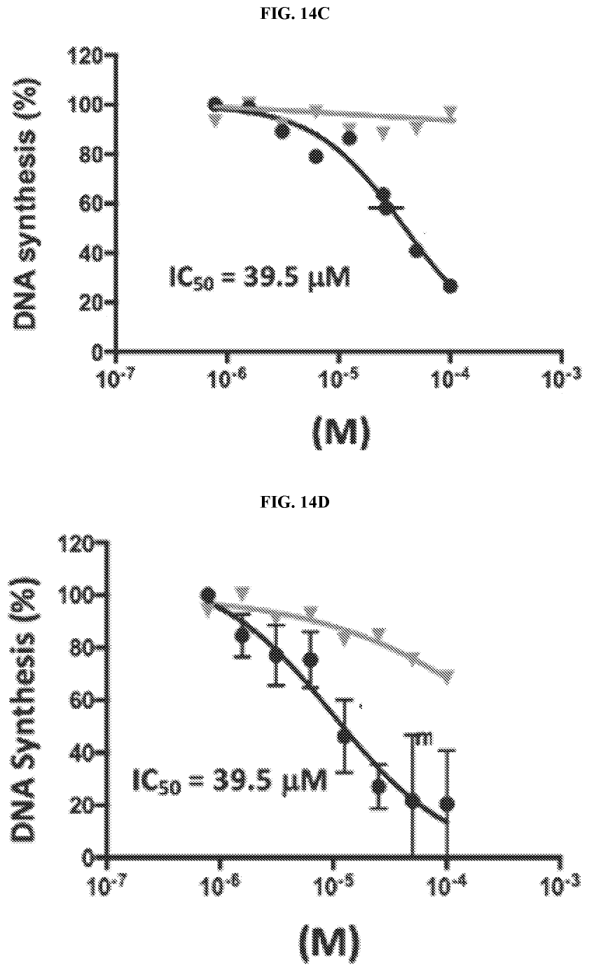

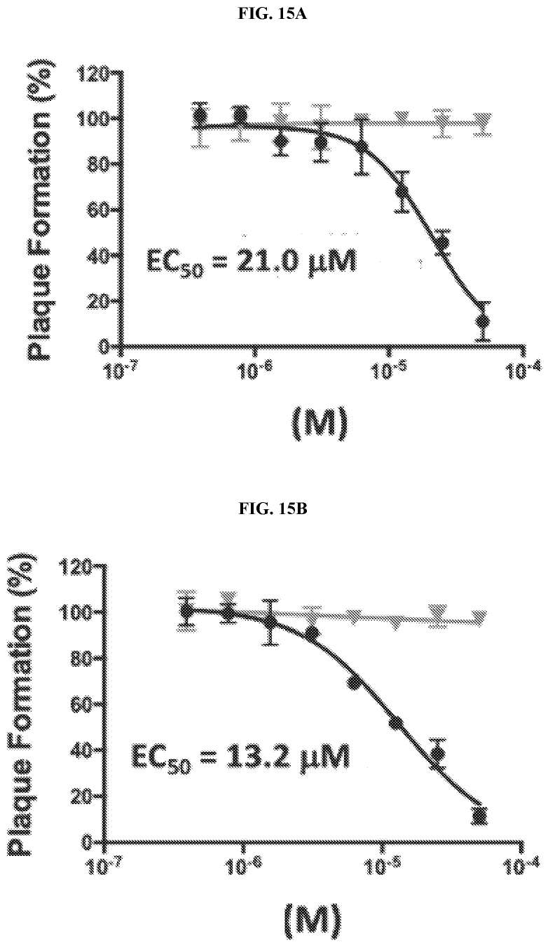

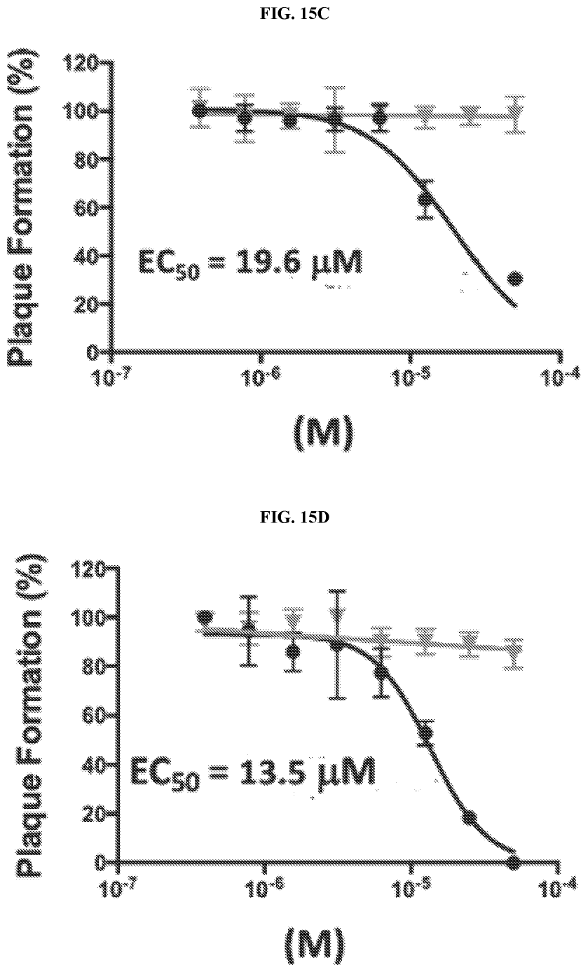

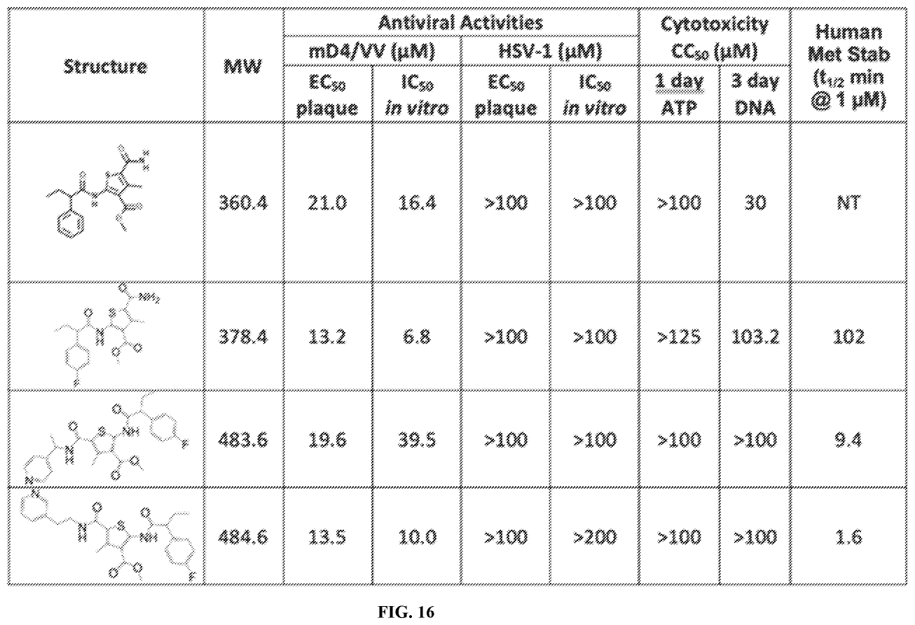

Compound 1 was examined for promiscuous DNA-binding, as this could have falsely contributed to the observed inhibition of the in vitro DNA synthesis. Measured by isothermal titration calorimetry (ITC), the use of sheared DNA served as a source of random duplex DNA and a 15-mer poly d(TC) oligomer as a single-stranded DNA source. The injections of either duplex or single-stranded DNA into the sample containing compound 1 produced heat traces that showed no appreciable total heats and a lack of signal saturation, both attributes equivalent to the heats of dilution generated by the buffer injections alone (FIG. 13), thus ruling out DNA-binding by compound 1 to both DNA types. As a positive control, appreciable heats and saturating signals can be observed for the DNA-intercalating ethidium bromide binding to duplex DNA (FIG. 13). Taken together, various approaches have supported the binding of compound 1 to D4, in addition to demonstrating the lack of binding promiscuity to unrelated proteins and DNA.

### Example 4: Synthesis of Selected Compounds of the Invention

### Compound 22. 3-Methyl-5-(2-phenyl-butyrylamino)-thiophene-2-carboxylic acid methyl ester

2-Phenylbutyric acid (0.178 g, 1.08 mmol) was dissolved in anhydrous dichloromethane (2 mL). Oxalyl chloride (2.0 M in dichloromethane, 0.54 mL, 1.08 mmol) followed by one drop of dimethylformamide were added. The reaction was stirred at room temperature for two hours then evaporated to dryness. The residue was dissolved in anhydrous pyridine (2 mL). 5-Amino-3-methyl-thiophene-2-carboxylic acid methyl ester hydrochloride (0.075 g, 0.36 mmol) and 4-dimethylaminopyridine (0.004 g, 0.036 mmol) were added. The reaction was stirred for 16 hours at room temperature. It was then diluted with brine and extracted with ethyl acetate. The extracts were concentrated and chromatographed (12 g column, 10 to 40% ethyl acetate in hexanes) to give 109 mg of the product as a white solid. MS: [M+H]⁺ 318.15.

### Compound 29. Methyl 2-(2-phenylbutanamido)-5-carbamoyl-4-methylthiophene-3-carboxylate

2-Phenylbutanoic acid (80 mg, 0.44 mmol) was taken up into 3 mL dry DCM under a N2 atmosphere. 1.2 equivalents of oxalyl chloride (45 μL, 53 mmol) was added to the solution along with 1 drop of dry DMF. The reaction stirred at room temperature for one hour. The solvent was removed under vacuum and the material placed under high vacuum for one hour. The acyl chloride was taken up into 3 mL pyridine and 5-amino-3-methyl-4-propionylthiophene-2-carboxamide (25 mg, 0.13 mmol) was added. A catalytic amount of DMAP was added, and the reaction stirred at room temperature overnight. EtOAc was added and then washed with 1N HCl, sat. sodium bicarbonate, and brine. The solvent was removed under vacuum and purified by normal phase chromatography (0-10% MeOH/DCM). 9 mg (19% yield) of the solid was produced. MS [M+H]⁺=361.

### Compound 32. 4-Methyl-2-(2-phenyl-butyrylamino)-thiophene-3-carboxylic acid methyl ester

A mixture of 2-phenyl-butyric acid (266 mg, 1.62 mmol) in 2M oxalyl chloride in dichloromethane (0.77 mL, 1.54 mmol) was treated with DMF (3 drops). The reaction was stirred for 2 hours, then was concentrated. The residue was treated with pyridine (0.5 mL), 2-amino-4-methyl-thiophene-3-carboxylic acid ethyl ester (100 mg, 0.54 mmol), and DMAP (5 mg). The mixture was stirred for 16 hours. The mixture was treated with ethyl acetate (30 mL), then was washed with 1N HCl (2×20 mL), water (5 mL), and brine (1 mL), dried (Na₂SO₄), and concentrated. The crude material was purified by column on silica (0-10% ethyl acetate: hexane). The mostly pure material was dissolved in methanol (2 mL), and was treated with 6N NaOH (0.2 mL, 1.2 mmol). The reaction was stirred for 2 hours, then was concentrated. The residue was treated with water (5 mL), and ethyl acetate (20 mL). The organic layer was washed with brine (1 mL), dried (Na2SO₄), and concentrated. The crude material was purified by column on silica (0-10% ethyl acetate: hexane), to give 4-methyl-2-(2-phenyl-butyrylamino)-thiophene-3-carboxylic acid methyl ester (28 mg, 16%) as a clear tacky gum. MS: [M+H]⁺ 318.

### Compound 33. 5-Carbamoyl-2-[2-(1,3-dioxo-1,3-dihydro-isoindol-2-yl)-3-phenyl-propionylamino]-4-methyl-thiophene-3-carboxylic acid methyl ester

2-(1,3-Dioxo-1,3-dihydro-isoindol-2-yl)-3-phenyl-propionic acid (0.413 g, 1.40 mmol) was dissolved in anhydrous dichloromethane (6 mL). Oxalyl chloride (2.0 M in dichloromethane, 0.70 mL, 1.40 mmol) followed by one drop of dimethylformamide were added. The reaction was stirred at room temperature for one hour then evaporated to dryness. The residue was dissolved in anhydrous pyridine (5 mL). 2-Amino-5-carbamoyl-4-methyl-thiophene-3-carboxylic acid methyl ester (0.100 g, 0.467 mmol) and 4-dimethylaminopyridine (0.006 g, 0.047 mmol) were added. The reaction was stirred for 16 hours at room temperature. It was then diluted with saturated aqueous sodium bicarbonate and extracted with ethyl acetate. The extracts were concentrated and chromatographed (12 g column, 10 to 40% ethyl acetate in hexanes) to give a white solid. The solid was dissolved in diethyl ether and hydrogen chloride (1.0 M in diethyl ether) was added. The resulting suspension was evaporated to dryness to give the hydrochloride salt as a white solid (58 mg). MS: [M+H]⁺ 492.6.

### Compound 34. N-(4-methyl-thiophen-2-yl)-2-phenyl-butyramide

A mixture of 2-phenyl-butyric acid (73 mg, 0.44 mmol), EDC·HCl (126 mg, 0.66 mmol), and HOAt (90 mg, 0.66 mmol) in DMF (2 mL) was stirred for 5 minutes, then 4-methyl-thiophen-2-ylamine (50 mg, 0.44 mmol) was added. The reaction was stirred for 3 days. The mixture was treated with water (30 mL), then was extracted with ethyl acetate (2×20 mL). The combined organic extracts was washed with water (2×10 mL), and brine (1 mL), dried (Na2SO₄), and concentrated. The crude material was purified by column on silica (0-30% ethyl acetate: hexane), to give N-(4-methyl-thiophen-2-yl)-2-phenyl-butyramide (15 mg, 13%) as a gray solid. MS: [M+H]⁺ 260.

### Compound 35. 5-Carbamoyl-2-[(indane-1-carbonyl)-amino]-4-methyl-thiophene-3-carboxylic acid methyl ester

A mixture of indan-1-carboxylic acid (131 mg, 0.81 mmol) in 2M oxalyl chloride in dichloromethane (0.39 mL, 0.78 mmol) was treated with DMF (2 drops). The reaction was stirred for 2 hours, then was concentrated. The residue was treated with pyridine (0.5 mL), 2-amino-5-carbamoyl-4-methyl-thiophene-3-carboxylic acid methyl ester (58 mg, 0.27 mmol), and DMAP (5 mg). The mixture was stirred for 16 hours. The mixture was treated with ethyl acetate (30 mL), then was washed with 1N HCl (2×20 mL), water (5 mL), and brine (1 mL), dried (Na2SO₄), and concentrated. The crude material was purified by column on silica (0-100% ethyl acetate: hexane), to give 5-carbamoyl-2-[(indane-1-carbonyl)-amino]-4-methyl-thiophene-3-carboxylic acid methyl ester (47 mg, 49%) as a white solid. MS: [M+H]⁺ 359.

### Compound 36. 5-Carbamoyl-4-methyl-2-[(1-phenyl-cyclopropanecarbonyl)-amino]-thiophene-3-carboxylic acid methyl ester

A mixture of 1-phenyl-cyclopropanecarboxylic acid (262 mg, 1.62 mmol) in 2M oxalyl chloride in dichloromethane (0.77 mL, 1.54 mmol) was treated with DMF (3 drops). The reaction was stirred for 2 hours, then was concentrated. The residue was treated with pyridine (0.5 mL), 2-amino-5-carbamoyl-4-methyl-thiophene-3-carboxylic acid methyl ester (116 mg, 0.54 mmol), and DMAP (5 mg). The mixture was stirred for 16 hours. The mixture was treated with ethyl acetate (30 mL), then was washed with 1N HCl (2×20 mL), water (5 mL), and brine (1 mL), dried (Na2SO₄), and concentrated. The crude material was purified by column on silica (0-100% ethyl acetate: hexane), to give 5-carbamoyl-4-methyl-2-[(1-phenyl-cyclopropanecarbonyl)-amino]-thiophene-3-carboxylic acid methyl ester (13 mg, 7%) as an off-white solid. MS: [M+H]⁺ 359.

### Compound 39. N-(3-Cyano-4,5-dimethyl-thiophen-2-yl)-2-phenyl-N-(2-phenyl-butyryl)-butyramide

A mixture of 2-phenyl-butyric acid (266 mg, 1.62 mmol) in 2M oxalyl chloride in dichloromethane (0.77 mL, 1.54 mmol) was treated with DMF (3 drops). The reaction was stirred for 2 hours, then was concentrated. The residue was dissolved in dichloromethane (2.5 mL), then was treated with diisopropylethylamine (137 mg, 1.08 mmol), and 2-amino-4,5-dimethyl-thiophene-3-carbonitrile (82 mg, 0.54 mmol). The reaction was stirred for 16 hours. The mixture was treated with water (30 mL), then was extracted with ethyl acetate (2×20 mL). The combined organic extracts were washed with water (2×10 mL), and brine (1 mL), dried (Na2SO₄), and concentrated. The crude material was purified by column on silica (0-30% ethyl acetate: hexane), to give N-(3-cyano-4,5-dimethyl-thiophen-2-yl)-2-phenyl-N-(2-phenyl-butyryl)-butyramide (25 mg, 10%) as a gray solid. MS: [M+H]⁺ 445.

### Compound 47. 5-[Bis-(1,2,3,4-tetrahydro-naphthalene-1-carbonyl)-amino]-4-cyano-3-methyl-thiophene-2-carboxylic acid amide

A mixture of 1,2,3,4-tetrahydro-naphthalene-1-carboxylic acid (211 mg, 1.20 mmol) in 2M oxalyl chloride in dichloromethane (0.60 mL, 1.20 mmol) was treated with DMF (3 drops). The reaction was stirred for 2 hours, then was concentrated. The residue was treated with pyridine (0.5 mL), 5-amino-4-cyano-3-methyl-thiophene-2-carboxylic acid amide (72 mg, 0.40 mmol), and DMAP (5 mg). The mixture was stirred for 16 hours. The mixture was treated with ethyl acetate (30 mL), then was washed with 1N HCl (2×20 mL), water (10 mL), and brine (1 mL), dried (Na₂SO₄), and concentrated. The crude material was purified by column on silica (0-100% ethyl acetate: hexane), to give 5-[bis-(1,2,3,4-tetrahydro-naphthalene-1-carbonyl)-amino]-4-cyano-3-methyl-thiophene-2-carboxylic acid amide (11 mg, 6%) as a clear tacky gum. MS: [M+H]⁺ 498.

### Compound 50. 5-[(5-Chloro-indane-1-carbonyl)-amino]-4-cyano-3-methyl-thiophene-2-carboxylic acid amide

A mixture of 6-chloro-indan-1-carboxylic acid (125 mg, 0.64 mmol) in 2M oxalyl chloride in dichloromethane (0.32 mL, 0.64 mmol) was treated with DMF (2 drops). The reaction was stirred for 2 hours, then was concentrated. The residue was treated with pyridine (0.35 mL), 5-amino-4-cyano-3-methyl-thiophene-2-carboxylic acid amide (58 mg, 0.32 mmol), and DMAP (5 mg). The mixture was stirred for 16 hours. The mixture was treated with water, then was extracted with ethyl acetate (2×20 mL). The combined organic extracts was washed with water (2×10 mL), and brine (1 mL), dried (Na₂SO₄), and concentrated. The crude material was purified by column on silica (0-100% ethyl acetate: hexane), to give 5-[(5-chloro-indane-1-carbonyl)-amino]-4-cyano-3-methyl-thiophene-2-carboxylic acid amide (101 mg, 88%) as a white solid. MS: [M+H]⁺ 360.

### Compound 57. 5-(2-{Bis[(4-methoxyphenyl)methyl]amino}-3-phenylpropanamido)-4-cyano-3-methylthiophene-2-carboxamide

tert-Butyl 2-amino-3-phenylpropanoate·HCl (600 mg, 2.32 mmol), 1-(bromomethyl)-4-methoxybenzene (0.85 mL, 5.8 mmol), and DIEA (1.4 mL, 8.1 mmol) were taken up into 4 mL DMF and stirred at room temperature overnight. EtOAc was added and washed with sat. NaHCO₃ then brine. Removed solvent under vacuum then purified with normal phase chromatography (0-30% EtOAc/Hex). 900 mg (84% yield) produced.

tert-Butyl 2-(bis(4-methoxybenzyl)amino)-3-phenylpropanoate (900 mg, 2.0 mmol) was taken up into 3 mL DCM and 3 mL TFA. The reaction was stirred at room temperature overnight. The solvent was removed under vacuum and taken up into EtOAc. The organic layer was washed with sat. NaHCO₃ then brine. The solvent was removed under vacuum to produce 740 mg of 2-(bis(4-methoxybenzyl)amino)-3-phenylpropanoic acid (335 mg, 0.83 mmol), which was treated with fluoro-N,N,N′,N′-tetramethylformamidinium hexafluorophosphate (219 mg, 0.83 mmol), and triethylamine (0.58 mL, 4.2 mmol) in 4 mL DCM at room temperature for one hour. The solvent was removed under vacuum and taken up into 3 mL pyridine. 5-Amino-4-cyano-3-methylthiophene-2-carboxamide (50 mg, 0.28 mmol) and a catalytic amount of DMAP were added and the reaction stirred overnight. EtOAc was added and then washed with 1N HCl, sat. sodium bicarbonate, and brine. The solvent was removed under vacuum and purified by normal phase chromatography (0-10% MeOH/DCM). 85 mg (53% yield). MS [M+H]⁺=569.

### Compound 68. N-(4-Cyano-3-methyl-isothiazol-5-yl)-2-phenyl-butyramide

A mixture of 5-amino-3-methyl-isothiazole-4-carbonitrile (50 mg, 0.36 mmol), and DMAP (5 mg) in pyridine (0.5 mL), was treated with 2-phenyl-butyryl chloride (66 mg, 0.36 mmol. The mixture was stirred for 16 hours. The mixture was treated with ethyl acetate (30 mL), then was washed with 1N HCl (2×20 mL), water (10 mL), and brine (1 mL), dried (Na₂SO₄), and concentrated. The crude material was purified by column on silica (0-100% ethyl acetate: hexane), to give N-(4-cyano-3-methyl-isothiazol-5-yl)-2-phenyl-butyramide (60 mg, 59%) as a white solid. MS: [M+H]⁺ 286.

### Compound 75. 5-(2-(4-Nitrophenyl)butanamido)-4-cyano-3-methylthiophene-2-carboxamide

2-(4-Nitrophenyl)butanoic acid (0.20 g, 1.0 mmol) was taken up into 5 mL dry DCM under a N2 atmosphere. 1.2 eq of oxalyl chloride (0.10 mL, 1.2 mmol) was added to the solution along with 1 drop of dry DMF. The reaction stirred at room temperature for one hour. The solvent was removed under vacuum and the material placed under high vacuum for one hour. The acyl chloride was taken up into 3 mL pyridine and 5-amino-4-cyano-3-methylthiophene-2-carboxamide (0.14 g, 0.80 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified by normal phase chromatography (0-10% MeOH/DCM). 95 mg (32% yield) of the solid was produced. MS [M+H]⁺=373.

### Compound 82. Methyl 5-(2-phenylbutanamido)-4-cyanothiophene-2-carboxylate

Methyl 5-amino-4-cyanothiophene-2-carboxylate (100 mg, 0.55 mmol) was taken up into 3 mL pyridine. 2-phenylbutanoyl chloride (118 μL, 0.72 mmol) was added along with a catalytic amount of DMAP. The reaction stirred overnight. EtOAc was added and then washed with 1N HCl, sat. sodium bicarbonate, and brine. The solvent was removed under vacuum and purified by HPLC. 75 mg (43% yield) of methyl 5-(2-phenylbutanamido)-4-cyanothiophene-2-carboxylate as a solid TFA salt was produced. MS [M+H]⁺=329.

### Compound 87. Indan-1-carboxylic acid (4-cyano-3-methyl-isothiazol-5-yl)-amide

A mixture of indan-1-carboxylic acid (26 mg, 0.16 mmol), 5-amino-3-methyl-isothiazole-4-carbonitrile (45 mg, 0.32 mmol), and trimethylamine (50 mg, 0.48 mmol) in ethyl acetate (1.5 mL) was treated with a 50% solution of 1-propanephosphonic acid cyclic anhydride in ethyl acetate (0.19 mL, 0.32 mmol). The reaction was heated in a microwave reactor at 160° C. for 15 minutes. The reaction was diluted with ethyl acetate (15 mL), then was washed with saturated aqueous sodium bicarbonate (10 mL), water (5 mL), and brine (1 mL), dried (Na₂SO₄), and concentrated. The crude material was purified by preparative HPLC, to give indan-1-carboxylic acid (4-cyano-3-methyl-isothiazol-5-yl)-amide as the TFA salt (40 mg, 52%) as a pale yellow solid. MS: [M+H]⁺ 284.

### Compound 89. 3-Methyl-5-(2-phenyl-butyrylamino)-isothiazole-4-carboxylic acid methyl ester

A mixture of 5-amino-3-methyl-isothiazole-4-carboxylic acid methyl ester (67 mg, 0.36 mmol), and DMAP (5 mg) in pyridine (0.5 mL), was treated with 2-phenyl-butyryl chloride (66 mg, 0.36 mmol. The mixture was stirred for 16 hours. The mixture was treated with ethyl acetate (30 mL), then was washed with 1N HCl (2×10 mL), water (5 mL), and brine (1 mL), dried (Na₂SO₄), and concentrated. The crude material was purified by column on silica (0-50% ethyl acetate: hexane). The mostly pure material was dissolved in methanol (1 mL), and was treated with 6N NaOH (50 μL). The reaction was stirred for 2 hours, then was concentrated. The residue was treated with ethyl acetate (20 mL), then was washed with saturated aqueous sodium carbonate (10 mL), water (5 mL), and brine (1 mL), dried (Na₂SO₄), and concentrated. The crude material was purified by preparative HPLC, to give 3-methyl-5-(2-phenyl-butyrylamino)-isothiazole-4-carboxylic acid methyl ester (8 mg, 7%) as a beige solid. MS: [M+H]⁺ 319.

### Compound 90. N-(4-Cyano-isothiazol-5-yl)-2-phenyl-butyramide

A mixture of 5-amino-isothiazole-4-carbonitrile (45 mg, 0.36 mmol), and DMAP (5 mg) in pyridine (0.5 mL), was treated with 2-phenyl-butyryl chloride (66 mg, 0.36 mmol. The mixture was stirred for 16 hours. The mixture was treated with ethyl acetate (30 mL), then was washed with 1N HCl (2×20 mL), water (10 mL), and brine (1 mL), dried (Na₂SO₄), and concentrated. The crude material was purified by column on silica (0-100% ethyl acetate: hexane), to give N-(4-cyano-isothiazol-5-yl)-2-phenyl-butyramide (81 mg, 83%) as a white solid. MS: [M+H]⁺ 272.

### Compound 95. Methyl 2-(2-(2-methoxyphenyl)butanamido)-5-carbamoyl-4-methylthiophene-3-carboxylate

2-(2-Methoxyphenyl)acetic acid (0.70 g, 4.2 mmol) was taken up into 16 mL of dry THF. The solution was cooled to −78° C. via dry ice/acetone bath. 2.1 equivalents of 2.0 M n-butyl lithium (4.4 mL, 0.010 mol) in hexanes was added dropwise. The solution was stirred for two hours at 0° C. 1.5 eq. of iodoethane (0.50 mL, 6.3 mmol) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 420 mg (51% yield) of a white solid was produced.

2-(2-Methoxyphenyl)butanoic acid (70 mg, 0.36 mmol) was taken up into 1 mL dry DCM under a N2 atmosphere. 1.2 eq of oxalyl chloride (34 μL, 0.4 mmol) was added to the solution along with 1 drop of dry DMF. The reaction stirred at room temperature for one hour. The solvent was removed under vacuum and the material placed under high vacuum for one hour. The acyl chloride was taken up into 2 mL pyridine and methyl 2-amino-5-carbamoyl-4-methylthiophene-3-carboxylate (40 mg, 0.18 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified by HPLC. 5 mg (8% yield) of methyl 2-(2-(2-methoxyphenyl) butanamido)-5-carbamoyl-4-methylthiophene-3-carboxylate as a solid TFA salt was produced. MS [M+H]⁺=391.

### Compound 96. Methyl 2-(2-(3-methoxyphenyl)butanamido)-5-carbamoyl-4-methylthiophene-3-carboxylate

2-(3-Methoxyphenyl)acetic acid (0.70 g, 4.2 mmol) was taken up into 16 mL of dry THF. The solution was cooled to −78° C. via dry ice/acetone bath. 2.1 equivalents of 2.0 M n-Butyl lithium (4.4 mL, 0.010 mol) in hexanes was added dropwise. The solution was stirred for two hours at 0° C. 1.5 eq. of iodoethane (0.50 mL, 6.3 mmol) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 420 mg (51% yield) of 2-(3-methoxyphenyl)butanoic acid as a white solid was produced.

2-(3-Methoxyphenyl)butanoic acid (70 mg, 0.36 mmol) was taken up into 1 mL dry DCM under a N2 atmosphere. 1.2 eq of oxalyl chloride (34 μL, 0.4 mmol) was added to the solution along with 1 drop of dry DMF. The reaction stirred at room temperature for one hour. The solvent was removed under vacuum and the material placed under high vacuum for one hour. The acyl chloride was taken up into 2 mL pyridine and methyl 2-amino-5-carbamoyl-4-methylthiophene-3-carboxylate (40 mg, 0.18 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified to afford 28 mg (44% yield) of methyl 2-(2-(3-methoxyphenyl) butanamido)-5-carbamoyl-4-methylthiophene-3-carboxylate as a solid TFA salt. MS [M+H]⁺=391.

### Compound 97. Methyl 2-(2-(4-methoxyphenyl)butanamido)-5-carbamoyl-4-methylthiophene-3-carboxylate

2-(4-Methoxyphenyl)butanoic acid (70 mg, 0.36 mmol) was taken up into 1 mL dry DCM under a N2 atmosphere. 1.2 eq of oxalyl chloride (34 μL, 0.4 mmol) was added to the solution along with 1 drop of dry DMF. The reaction was stirred at room temperature for one hour. The solvent was removed under vacuum and the material placed under high vacuum for one hour. The acyl chloride was taken up into 2 mL pyridine and methyl 2-amino-5-carbamoyl-4-methylthiophene-3-carboxylate (40 mg, 0.18 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified by HPLC. 14 mg (22% yield) of methyl 2-(2-(4-methoxyphenyl) butanamido)-5-carbamoyl-4-methylthiophene-3-carboxylate as a solid TFA salt was produced. MS [M+H]⁺=391.

### Compound 98. Methyl 2-(2-(3-fluorophenyl)butanamido)-5-carbamoyl-4-methylthiophene-3-carboxylate

2-(3-Fluorophenyl)acetic acid (0.50 g, 3.2 mmol) was taken up into 16 mL of dry THF. The solution was cooled to −78° C. via dry ice/acetone bath. 2.1 equivalents of 2.0 M n-butyllithium (3.4 mL, 0.067 mol) in hexanes was added dropwise. The solution was stirred for two hours at 0° C. 1.5 eq. of iodoethane (0.39 mL, 4.8 mmol) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 310 mg (53% yield) of 2-(3-flourophenyl)butanoic acid as a solid was produced.

2-(3-Flourophenyl)butanoic acid (68 mg, 0.36 mmol) was taken up into 1 mL dry DCM under a N2 atmosphere. 1.2 eq of oxalyl chloride (34 μL, 0.4 mmol) was added to the solution along with 1 drop of dry DMF. The reaction stirred at room temperature for one hour. The solvent was removed under vacuum and the material placed under high vacuum for one hour. The acyl chloride was taken up into 2 mL pyridine and methyl 2-amino-5-carbamoyl-4-methylthiophene-3-carboxylate (40 mg, 0.18 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified by HPLC. 14 mg (22% yield) of methyl 2-(2-(3-fluorophenyl) butanamido)-5-carbamoyl-4-methylthiophene-3-carboxylate as a solid TFA salt was produced. MS [M+H]⁺=379.

### Compound 99. 5-Carbamoyl-2-[2-(4-fluoro-phenyl)-butyrylamino]-4-methyl-thiophene-3-carboxylic acid methyl ester

2-(4-Fluorophenyl)acetic acid (6.16 g, 0.040 mol) was taken up into 160 mL of dry THF. The solution was cooled to −78° C. via dry ice/acetone bath. 2.1 equivalents of 2.5 M n-butyllithium (33.6 mL, 0.084 mol) in hexanes was added dropwise. The solution was stirred for two hours at 0° C. 1.2 eq. of iodoethane (3.8 mL, 0.048 mol) was added slowly and the reaction was stirred at room temperature overnight. The reaction was quenched with 40 mL water and the volatiles were removed under vacuum. 1 N HCl was added to the solution and the product was extracted with diethyl ether twice. The crude material was purified using normal phase chromatography (0-5% MeOH/DCM). 5.8 g (80% yield) of a white solid, 2-(4-fluorophenyl) butanoic acid, was produced.

2-(4-Fluorophenyl)butanoic acid (2.55 g, 0.014 mol) was taken up into 50 mL dry DCM under a N2 atmosphere. 1.1 eq of oxalyl chloride (1.32 mL, 0.0154 mol) was added to the solution along with 4 drops of dry DMF. The reaction stirred at room temperature for one hour. The reaction was complete by quenching one drop in MeOH and observing the methyl ester only by LC/MS. The solvent was removed under vacuum and the material placed under high vacuum for one hour. The product, 2-(4-fluorophenyl)butanoyl chloride, was taken on as is.

2-(4-Fluorophenyl)butanoyl chloride (2.8 g, 0.014 mol) and methyl 2-amino-5-carbamoyl-4-methylthiophene-3-carboxylate (2.35 g, 0.011 mol) were taken up into 50 mL pyridine. A catalytic amount of DMAP was added and the reaction stirred at room temperature overnight. 300 mL of EtOAc was added and then washed with 1N HCl, sat. sodium bicarbonate, and brine. The solvent was remove under vacuum and purified by normal phase chromatography (0-50% EtOAc/DCM). 1.6 g (30% yield) of 5-carbamoyl-2-[2-(4-fluoro-phenyl)-butyrylamino]-4-methyl-thiophene-3-carboxylic acid methyl ester as a tan solid was produced.

### Compound 106. 5-(2-Phenyl-butyrylamino)-[1,2,3]thiadiazole-4-carboxylic acid ethyl ester

Ethyl 5-amino-1,2,3-thiadiazole-4-carboxylate (0.169 g, 0.98 mmol) was dissolved in pyridine (5 mL) and then 2-phenylbutyryl chloride (0.196 g, 1.07 mmol) was added. The mixture was stirred at room temperature for one hour and then diluted with water and extracted with dichloromethane. The extracts were concentrated and chromatographed (12 g column, 10 to 40% ethyl acetate in hexanes) to give a white solid (172 mg). MS: [M+H]⁺ 320.05.

### Compound 109. 2-Phenyl-N-[4-(4-trifluoromethyl-phenyl)-[1,2,3]thiadiazol-5-yl]-butyramide

4-(4-Trifluoromethyl-phenyl)-[1,2,3]thiadiazol-5-ylamine (0.073 g, 0.30 mmol) was dissolved in a mixture of dichloromethane (2 mL) and pyridine (0.5 mL). 2-Phenylbutyryl chloride (0.065 g, 0.36 mmol) was added and the mixture was stirred for one hour at room temperature. The reaction was diluted with water and extracted with dichloromethane. The extracts were concentrated and chromatographed (12 g column, 0 to 30% ethyl acetate in hexanes) to give a white foam (48 mg). MS: [M+H]⁺ 392.

### Compound 110. 2-(2-Phenylbutanamido)-N-(4-chlorobenzyl)thiophene-3-carboxamide

Methyl 2-(2-phenylbutanamido)thiophene-3-carboxylate (0.45 g, 1.5 mmol) was taken up into 15 mL MeOH/THF/H₂O (1/1/1) and lithium hydroxide (623 mg, 15 mmol) was added. The reaction stirred at room temperature overnight. The volatiles were removed under vacuum and the solution was acidified with 4 mL 1N HCl. The precipitate was filtered off, and 320 mg of the resulting 2-(2-phenylbutanamido)thiophene-3-carboxylic acid (50 mg, 0.17 mmol) was treated with 4-chlorobenzylamine (42 μL, 0.34 mmol), EDCI (67 mg, 0.34 mmol), HOBt (53 mg, 0.34 mmol), and DIEA (89 μL, 0.51 mmol) in 1.0 mL DMF and stirred at room temperature overnight. The reaction was purified by preparative HPLC to yield 2-(2-phenylbutanamido)-N-(4-chlorobenzyl)thiophene-3-carboxamide (45 mg, 64% yield) as a TFA salt. MS [M+H]⁺=413.

### Compound 111. 4-Pyridin-2-yl-piperazine-1-carboxylic acid [3-(4-chloro-benzyl carbamoyl)-thiophen-2-yl]-amide

Methyl 2-aminothiophene-3-carboxylate (2.47 g, 0.016 mol) and phenyl chloroformate (2.4 mL, 0.019 mol) were taken up into 70 mL dry THF under N2 atmosphere. Pyridine (1.9 mL, 0.023) and a catalytic amount of DMAP were added and the reaction stirred at room temperature overnight. 300 mL of EtOAc was added and then washed with 1N HCl, sat. sodium bicarbonate, and brine. The solvent was removed under vacuum. 4.35 g (quantitative) of phenyl 3-(methoxycarbonyl)thiophen-2-ylcarbamate was produced and taken on as is.

Phenyl 3-(methoxycarbonyl)thiophen-2-ylcarbamate (4.35 g, 0.016 mol) and 1-(pyridin-2-yl)piperazine (2.95 mL, 0.020 mol) were taken up into 60 mL DMF. DIEA (6.4 mL, 0.037 mol) was added and the reaction was stirred at room temperature overnight. EtOAc was added and washed with sat. NaHCO₃ and brine. The crude material was purified by normal phase chromatography (0-10% MeOH/DCM). 4.8 g (86% yield) of the solid, methyl 2-(4-(pyridin-2-yl)piperazine-1-carboxamido)thiophene-3-carboxylate, was produced.

Methyl 2-(4-(pyridin-2-yl)piperazine-1-carboxamido)thiophene-3-carboxylate (4.75 g, 0.014 mol) was taken up into 60 mL MeOH/THF/H₂O (1/1/1) and LiOH·H₂O (5.8 g, 0.14 mol) was added. The reaction was stirred at room temperature overnight. The volatiles were removed under vacuum and 150 mL 1 N HCl was added. The product 2-(4-(pyridin-2-yl)piperazine-1-carboxamido)thiophene-3-carboxylic acid precipitated out of solution, and was filtered and washed with water (3.5 g, 77% yield).

2-(4-(Pyridin-2-yl)piperazine-1-carboxamido)thiophene-3-carboxylic acid (3.0 g, 9.04 mmol), 4-chloro benzyl amine (1.21 mL, 9.9 mmol), EDCI (2.1 g, 10.8 mmol), HOBt (1.65 g, 10.8 mmol), and DIEA (3.15 mL, 18 mmol) were taken up into 35 mL DMF and stirred at room temperature overnight. EtOAc was added and washed with sat. NaHCO₃ and brine. The crude material was purified by normal phase chromatography (0-10% MeOH/DCM). 3.0 g (73% yield) was produced. The solid was converted to the HCl salt by adding 3.5 mL of 4N HCl in dioxane to the material in 5 mL dioxane. The solution was stirred for 4 hours and then frozen. The solvent was removed by lyophilization to produce 3.25 g of 4-pyridin-2-yl-piperazine-1-carboxylic acid [3-(4-chloro-benzylcarbamoyl)-thiophen-2-yl]-amide.

### Compound 112. Methyl 2-(2-phenylbutanamido)thiophene-3-carboxylate

Methyl 2-aminothiophene-3-carboxylate (250 mg, 1.6 mmol) was taken up into 8 mL pyridine. 2-Phenylbutanoyl chloride (221 μL, 1.6 mmol) was added along with a catalytic amount of DMAP. The reaction stirred overnight. EtOAc was added and then washed with 1N HCl, sat. sodium bicarbonate, and brine. The solvent was removed under vacuum and purified by normal phase chromatography. 450 mg (93% yield) of methyl 2-(2-phenylbutanamido) thiophene-3-carboxylate as a solid was produced. MS [M+H]⁺=304.

### Compound 114. Methyl 2-(4-(pyridin-2-yl)piperazine-1-carboxamido)thiophene-3-carboxylate

Methyl 2-aminothiophene-3-carboxylate (2.47 g, 0.016 mol) and phenyl chloroformate (2.4 mL, 0.019 mol) were taken up into 70 mL dry THF under N2 atmosphere. Pyridine (1.9 mL, 0.023) and a catalytic amount of DMAP were added and the reaction stirred at room temperature overnight. EtOAc (300 mL) was added and the solution was washed with 1N HCl, sat. sodium bicarbonate, and brine. The solvent was remove under vacuum to yield phenyl 3-(methoxycarbonyl)thiophen-2-ylcarbamate (4.35 g, quantitative) which was taken on as is.

Phenyl 3-(methoxycarbonyl)thiophen-2-ylcarbamate (4.35 g, 0.016 mol) and 1-(pyridin-2-yl)piperazine (2.95 mL, 0.020 mol) were taken up into 60 mL DMF. DIEA (6.4 mL, 0.037 mol) was added and the reaction was stirred at room temperature overnight. EtOAc was added and washed with sat. NaHCO₃ and brine. The crude material was purified by normal phase chromatography (0-10% MeOH/DCM). 4.8 g (86% yield) of methyl 2-(4-(pyridin-2-yl)piperazine-1-carboxamido)thiophene-3-carboxylate as a solid was produced. MS [M+H]⁺=347.

### Compound 115. 2-(2-Phenylbutanamido)-N3-(4-chlorobenzyl)-4-methylthiophene-3,5-dicarboxamide

Methyl 2-(2-phenylbutanamido)-5-carbamoyl-4-methylthiophene-3-carboxylate (100 mg, 0.28 mmol) was taken up into 9 mL MeOH/THF/H₂O (1/1/1) and lithium hydroxide (116 mg, 2.8 mmol) was added. The reaction stirred at room temperature overnight. The volatiles were removed under vacuum and the solution was acidified with 4 mL 1N HCl. The precipitate was filtered off and 60 mg of 2-(2-phenylbutanamido)-5-carbamoyl-4-methylthiophene-3-carboxylic acid was taken on as is.

2-(2-Phenylbutanamido)-5-carbamoyl-4-methylthiophene-3-carboxylic acid (30 mg, 0.09 mmol), 4-chlorobenzylamine (15 mg, 0.11 mmol), EDCI (25 mg, 0.14 mmol), HOBt (21 mg, 0.14 mmol), and DIEA (47 μL, 0.23 mmol) were taken up into 0.5 mL DMF and stirred at room temperature overnight. The reaction was purified by preparative HPLC to yield 2-(2-phenylbutanamido)-N3-(4-chlorobenzyl)-4-methylthiophene-3,5-dicarboxamide (20 mg, 47% yield) as a TFA salt. MS [M+H]⁺=470.

### Compound 116. Methyl 2-(4-(pyridin-2-yl)piperazine-1-carboxamido)-5-carbamoyl-4-methylthiophene-3-carboxylate

2-Amino-5-carbamoyl-4-methylthiophene-3-carboxylate (50 mg, 0.23 mmol) was taken up into 3 mL pyridine. Phenyl chloroformate (95 μL, 0.75 mmol) was added along with a catalytic amount of DMAP. The reaction was stirred overnight. EtOAc was added and then washed with 1N HCl, sat. sodium bicarbonate, and brine. The solvent was removed under vacuum and purified by normal phase chromatography (0-10% MeOH/DCM) to afford phenyl 3-(methoxycarbonyl)-5-carbamoyl-4-methylthiophen-2-ylcarbamate (77 mg, quant yield).

Phenyl 3-(methoxycarbonyl)-5-carbamoyl-4-methylthiophen-2-ylcarbamate (77 mg, 0.23 mmol), 1-(pyridin-2-yl)piperazine (67 μL, 0.46 mmol), and triethyl amine (120 μL, 0.69 mmol) were taken up into 1 mL DMF and stirred at room temperature overnight. EtOAc was added and then washed with sat. sodium bicarbonate and brine. The solvent was removed under vacuum and purified by normal phase chromatography (0-10% MeOH/DCM) to yield methyl 2-(4-(pyridin-2-yl)piperazine-1-carboxamido)-5-carbamoyl-4-methylthiophene-3-carboxylate (85 mg, 90% yield). MS [M+H]⁺=404.

### Compound 118. 4-Pyridin-2-yl-piperazine-1-carboxylic acid [4-(4-trifluoromethyl-phenyl)-[1,2,3]thiadiazol-5-yl]-amide

Trifluoromethyl-phenyl)-[1,2,3]thiadiazol-5-ylamine (0.120 g, 0.49 mmol) was dissolved in a mixture of dichloromethane (5 mL) and pyridine (1 mL). Phenyl chloroformate (0.085 g, 0.54 mmol) was added and the mixture was stirred for two hours at room temperature. The reaction was diluted with water and extracted with dichloromethane. The extracts were concentrated and then dissolved in tetrahydrofuran (2 mL) in a pressure tube. 1-Pyridin-2-yl-piperazine (0.40 g, 0.49 mmol) was added, the tube was sealed, and then the mixture was heated to about 80° C. overnight. The reaction mixture was concentrated and the residue was chromatographed (12 g column, 10 to 40% ethyl acetate in hexanes) to give 4-pyridin-2-yl-piperazine-1-carboxylic acid [4-(4-trifluoromethyl-phenyl)-[1,2,3]thiadiazol-5-yl]-amide as white solid (52 mg). MS: [M+H]⁺ 434.96.

### Compound 119. 4-Pyridin-2-yl-piperazine-1-carboxylic acid (4-benzylcarbamoyl-[1,2,3]thiadiazol-5-yl)-amide

5-[(4-Pyridin-2-yl-piperazine-1-carbonyl)-amino]-[1,2,3]thiadiazole-4-carboxylic acid (0.046 g, 0.14 mmol) was suspended in anhydrous dimethylformamide. Diisopropylethylamine (0.089 g, 0.69 mmol) was added followed by benzotriazol-1-yl-oxytripyrrolidinophosphonium hexafluorophosphate (0.143 g, 0.275 mmol). The mixture was stirred for 15 minutes at room temperature and then benzylamine (0.029 g, 0.275 mmol) was added. The mixture was stirred for 16 hours at room temperature, diluted with brine, and extracted with ethyl acetate. The extracts were concentrated and chromatographed (12 g column, 25 to 50% ethyl acetate in hexanes) to give 4-pyridin-2-yl-piperazine-1-carboxylic acid (4-benzylcarbamoyl-[1,2,3]thiadiazol-5-yl)-amide as a white solid (5 mg). MS: [M+H]⁺ 423.96.

### Compound 123. 4-Phenyl-piperazine-1-carboxylic acid (4-benzylcarbamoyl-[1,2,3]thiadiazol-5-yl)-amide

5-Phenoxycarbonylamino-[1,2,3]thiadiazole-4-carboxylic acid ethyl ester (0.331 g, 1.13 mmol), 1-phenylpiperazine hydrochloride (0.247 g, 1.24 mmol), and diisopropylethylamine (0.219 g, 1.70 mmol) were dissolved in anhydrous tetrahydrofuran in a pressure tube. The tube was sealed and heated to about 80° C. for 16 hours. The reaction was cooled, diluted with saturated aqueous sodium bicarbonate, and extracted with ethyl acetate. The extracts were concentrated and chromatographed (12 g column, 20 to 50% ethyl acetate in hexanes) to give a white solid, 5-[(4-phenyl-piperazine-1-carbonyl)-amino]-[1,2,3]thiadiazole-4-carboxylic acid ethyl ester (398 mg).

5-[(4-Phenyl-piperazine-1-carbonyl)-amino]-[1,2,3]thiadiazole-4-carboxylic acid ethyl ester (0.393 g, 1.09 mmol) was dissolved in tetrahydrofuran (15 mL). Lithium hydroxide hydrate (0.228 g, 5.44 mmol) was added followed by enough water to dissolve most of the solid, about 4 mL. The reaction was stirred at room temperature for 16 hours. Aqueous hydrochloric acid (0.1 N) was added and the mixture was extracted repeatedly with ethyl acetate. The extracts were concentrated to a white solid which was chromatographed (12 g column, 2 to 10% methanol in dichloromethane) to give a white solid, 5-[(4-phenyl-piperazine-1-carbonyl)-amino]-[1,2,3]thiadiazole-4-carboxylic acid (212 mg).

5-[(4-Phenyl-piperazine-1-carbonyl)-amino]-[1,2,3]thiadiazole-4-carboxylic acid (0.051 g, 0.15 mmol) was suspended in anhydrous dimethylformamide. Diisopropylethylamine (0.040 g, 0.31 mmol) was added followed by benzotriazol-1-yl-oxytripyrrolidinophosphonium hexafluorophosphate (0.124 g, 0.23 mmol). The mixture was stirred for 15 minutes at room temperature and then benzylamine (0.025 g, 0.23 mmol) was added. The mixture was stirred for 16 hours at room temperature, diluted with brine, and extracted with ethyl acetate. The extracts were concentrated and chromatographed (12 g column, 25 to 50% ethyl acetate in hexanes) to give 4-phenyl-piperazine-1-carboxylic acid (4-benzylcarbamoyl-[1,2,3]thiadiazol-5-yl)-amide as a white solid (27 mg). MS: [M+H]⁺ 422.94.

### Compound 124. 4-Pyridin-2-yl-piperazine-1-carboxylic acid [4-(4-methyl-benzylcarbamoyl)-[1,2,3]thiadiazol-5-yl]-amide

5-[(4-Phenyl-piperazine-1-carbonyl)-amino]-[1,2,3]thiadiazole-4-carboxylic acid (0.051 g, 0.15 mmol) was suspended in anhydrous dimethylformamide. Diisopropylethylamine (0.040 g, 0.31 mmol) was added followed by benzotriazol-1-yl-oxytripyrrolidinophosphonium hexafluorophosphate (0.124 g, 0.23 mmol). The mixture was stirred for 15 minutes at room temperature and then 4-methylbenzylamine (0.028 g, 0.23 mmol) was added. The mixture was stirred for 16 hours at room temperature, diluted with brine, and extracted with ethyl acetate. The extracts were concentrated and chromatographed (12 g column, 25 to 50% ethyl acetate in hexanes) to give 4-pyridin-2-yl-piperazine-1-carboxylic acid [4-(4-methyl-benzylcarbamoyl)-[1,2,3]thiadiazol-5-yl]-amide as a white solid (31 mg) MS: [M+H]⁺ 437.96.

### Compound 127. Ethyl 5-(4-(pyridin-2-yl)piperazine-1-carboxamido)thiazole-4-carboxylate

Ethyl 5-aminothiazole-4-carboxylate (500 mg, 02.9 mmol) was taken up into 30 mL THF. Phenyl chloroformate (403 μL, 3.1 mmol) was added along with pyridine (290 μL, 3.5 mmol. The reaction stirred overnight. EtOAc was added and then washed with 1N HCl, sat. sodium bicarbonate, and brine. The solvent was removed under vacuum and purified by normal phase chromatography (0-10% MeOH/DCM). 850 mg (quant yield) of the solid was produced.

Phenyl 4-(ethoxycarbonyl)thiazol-5-ylcarbamate (400 mg, 1.3 mmol), 1-(pyridin-2-yl)piperazine (210 μL, 1.4 mmol), and DIEA (450 μL, 2.6 mmol) were taken up into 8 mL DMF and stirred at room temperature overnight. EtOAc was added and then washed with sat. sodium bicarbonate and brine. The solvent was removed under vacuum and purified by normal phase chromatography (0-10% MeOH/DCM) to give ethyl 5-(4-(pyridin-2-yl)piperazine-1-carboxamido)thiazole-4-carboxylate (430 mg, 92% yield) as a solid. MS [M+H]⁺=362.

### Compound 130. N-(3-(Benzylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.10 mmol), benzylamine (25 μL, 0.11 mmol), EDCI (38 mg, 0.20 mmol), HOBt (38 mg, 0.20 mmol), and DIEA (52 μL, 0.30 mmol) were taken up into 0.5 mL DMF and stirred at room temperature overnight. The reaction was purified by preparative HPLC to give N-(3-(benzylcarbamoyl) thiophen-2-yl)-4-(pyridin-2-yl)piperazine-1-carboxamide (40 mg, 75% yield) as a TFA salt. MS [M+H]⁺=422.

### Compound 131. 2-(4-(pyridin-2-yl)piperazine-1-carboxamido)-N3-benzyl-4-methylthiophene-3,5-dicarboxamide

2-(4-(Pyridin-2-yl)piperazine-1-carboxamido)-5-carbamoyl-4-methylthiophene-3-carboxylic acid (38 mg, 0.10 mmol), benzylamine (25 μL, 0.11 mmol), EDCI (38 mg, 0.20 mmol), HOBt (38 mg, 0.20 mmol), and DIEA (52 μL, 0.30 mmol) were taken up into 0.5 mL DMF and stirred at room temperature overnight. The reaction was purified by preparative HPLC to give 2-(4-(pyridin-2-yl)piperazine-1-carboxamido)-N3-benzyl-4-methylthiophene-3,5-dicarboxamide (44 mg, 74% yield) as the TFA salt. MS [M+H]⁺=479.

### Compound 132. [4-(4-Chloro-benzylcarbamoyl)-[1,2,3]thiadiazol-5-yl]-carbamic acid phenyl ester

Ethyl 5-amino-1,2,3-thiadiazole-4-carboxylate (0.328 g, 1.89 mmol) and triethylamine (0.287 g, 2.84 mmol) were dissolved in anhydrous dichloromethane (25 ml). Di-tert-butyl dicarbonate (0.413 g, 1.89 mmol) was added and then the reaction was stirred at room temperature for 16 hours. The mixture was diluted with saturated aqueous sodium bicarbonate and extracted with dichloromethane. The extracts were concentrated and chromatographed (20 g column, 10-50% ethyl acetate in hexanes) to give 5-tert-butoxycarbonylamino-[1,2,3]thiadiazole-4-carboxylic acid ethyl ester (352 mg).

5-tert-Butoxycarbonylamino-[1,2,3]thiadiazole-4-carboxylic acid ethyl ester (0.352 g, 1.29 mmol) and lithium hydroxide hydrate (0.162 g, 3.86 mmol) were combined in tetrahydrofuran (40 mL). Enough water was added to bring most of the solid into solution (8 mL). The mixture was stirred at room temperature overnight. TLC indicated incomplete reaction, so it was heated to reflux for 24 hours. The reaction was then acidified with 0.1 N aqueous hydrochloric acid and extracted with ethyl acetate. The extracts were concentrated and chromatographed (20 g column, 2 to 10% methanol in dichloromethane) to give 5-tert-butoxycarbonylamino-[1,2,3]thiadiazole-4-carboxylic acid (270 mg).

5-tert-Butoxycarbonylamino-[1,2,3]thiadiazole-4-carboxylic acid (0.270 g, 1.10 mmol) and diisopropylethylamine (0.284 g, 2.20 mmol) were dissolved in anhydrous dimethylformamide (10 mL). Benzotriazol-1-yl-oxytripyrrolidinophosphonium hexafluorophosphate (0.859 g, 1.65 mmol) was added and the mixture was stirred for 15 minutes at room temperature. 4-Chlorobenzylamine (0.234 g, 1.65 mmol) was added. The mixture was stirred for 16 hours at room temperature, diluted with brine, and extracted with ethyl acetate. The extracts were concentrated and chromatographed (40 g column, 100% dichloromethane) to give a white solid, [4-(4-chloro-benzylcarbamoyl)-[1,2,3]thiadiazol-5-yl]-carbamic acid tert-butyl ester (404 mg).

[4-(4-Chloro-benzylcarbamoyl)-[1,2,3]thiadiazol-5-yl]-carbamic acid tert-butyl ester (0.404 g, 1.10 mmol) was dissolved in a mixture of dichloromethane (25 mL) and trifluoroacetic acid (5 mL) and the solution was stirred at room temperature for 16 hours. The reaction was carefully basified with saturated aqueous sodium bicarbonate and then extracted with dichloromethane. The extracts were concentrated and chromatographed (40 g column, 0 to 5% methanol in dichloromethane) to give a white solid, 5-amino-[1,2,3]thiadiazole-4-carboxylic acid 4-chloro-benzylamide (291 mg).

5-Amino-[1,2,3]thiadiazole-4-carboxylic acid 4-chloro-benzylamide (0.291 g, 1.08 mmol) and triethylamine (0.164 g, 1.19 mmol) were dissolved in anhydrous dichloromethane. Phenyl chloroformate (0.187 g, 1.19 mmol) was added and the mixture was stirred at room temperature for 6 hours. Saturated aqueous sodium bicarbonate was added and the mixture was extracted with dichloromethane. The extracts were concentrated and chromatographed (20 g column, 10-50% ethyl acetate in hexanes) to give a solid, [4-(4-chloro-benzylcarbamoyl)-[1,2,3]thiadiazol-5-yl]-carbamic acid phenyl ester (351 mg).

### Compound 134. 5-(2-Phenyl-butyrylamino)-[1,2,3]thiadiazole-4-carboxylic acid 4-chloro-benzylamide

5-(2-Phenyl-butyrylamino)-[1,2,3]thiadiazole-4-carboxylic acid (0.073 g, 0.25 mmol) was suspended in anhydrous dimethylformamide (3 mL). Diisopropylethylamine (0.065 g, 0.50 mmol) was added followed by benzotriazol-1-yl-oxytripyrrolidinophosphonium hexafluorophosphate (0.196 g, 0.38 mmol). The mixture was stirred for 15 minutes at room temperature and then 4-chlorobenzylamine (0.040 g, 0.38 mmol) was added. The mixture was stirred for 16 hours at room temperature, diluted with brine, and extracted with ethyl acetate. The extracts were concentrated and chromatographed (12 g column, 20 to 50% ethyl acetate in hexanes) to give 5-(2-phenyl-butyrylamino)-[1,2,3]thiadiazole-4-carboxylic acid 4-chloro-benzylamide as a white solid (36 mg). MS: [M+H]⁺ 415.87.

### Compound 135. 5-(2-Phenyl-butyrylamino)-[1,2,3]thiadiazole-4-carboxylic acid benzylamide

Ethyl 5-amino-1,2,3-thiadiazole-4-carboxylate (0.169 g, 0.98 mmol) was dissolved in pyridine (5 mL). 2-Phenylbutyryl chloride (0.196 g, 1.07 mmol) was added. The mixture was stirred at room temperature for one hour, diluted with water, and extracted with dichloromethane. The extracts were concentrated and chromatographed (12 g column, 10 to 40% ethyl acetate in hexanes) to give a white solid, 5-(2-phenyl-butyrylamino)-[1,2,3]thiadiazole-4-carboxylic acid ethyl ester (172 mg).

5-(2-Phenyl-butyrylamino)-[1,2,3]thiadiazole-4-carboxylic acid ethyl ester (0.167 g, 0.52 mmol) was dissolved in tetrahydrofuran. Lithium hydroxide hydrate (0.110 g, 2.61 mmol) was added followed by enough water to get most of the solid to go into solution (4 mL). The mixture was heated to reflux for 16 hours, cooled to room temperature, and acidified with 0.1 N hydrochloric acid. The mixture was extracted with ethyl acetate. The extracts were concentrated and chromatographed (12 g column, 2 to 10% methanol in dichloromethane) to give a white solid 5-(2-phenyl-butyrylamino)-[1,2,3]thiadiazole-4-carboxylic acid (151 mg).

5-(2-Phenyl-butyrylamino)-[1,2,3]thiadiazole-4-carboxylic acid (0.073 g, 0.25 mmol) was suspended in anhydrous dimethylformamide (3 mL). Diisopropylethylamine (0.065 g, 0.50 mmol) was added followed by benzotriazol-1-yl-oxytripyrrolidinophosphonium hexafluorophosphate (0.196 g, 0.38 mmol). The mixture was stirred for 15 minutes at room temperature and then benzylamine (0.040 g, 0.38 mmol) was added. The mixture was stirred for 16 hours at room temperature, diluted with brine, and extracted with ethyl acetate. The extracts were concentrated and chromatographed (12 g column, 20 to 50% ethyl acetate in hexanes) to give 5-(2-phenyl-butyrylamino)-[1,2,3]thiadiazole-4-carboxylic acid benzylamide as a white solid (28 mg). MS: [M+H]⁺ 381.92.

### Compound 144. N-(3-((6-Chloropyridin-2-yl)methylcarbamoyl)thiophen-2-yl)-4-phenylpiperazine-1-carboxamide

2-(4-Phenylpiperazine-1-carboxamido)thiophene-3-carboxylic acid (33 mg, 0.1 mmol), (6-chloropyridin-3-yl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to give N-(3-((6-chloropyridin-2-yl)methyl carbamoyl)thiophen-2-yl)-4-phenylpiperazine-1-carboxamide (25 mg, 55% yield). MS [M+H]⁺=456.

### Compound 153. N-[4-(2-Fluoro-phenyl)-[1,2,3]thiadiazol-5-yl]-2-phenyl-butyramide

4-(2-Fluorophenyl)thiadiazole-5-amine (0.050 g, 0.26 mmol) and triethylamine (0.039 g, 0.39 mmol) were dissolved in anhydrous dichloromethane (2 mL). 2-Phenylbutyryl chloride (0.056 g, 0.31 mmol) was added. The reaction was stirred at room temperature for 3 hours. The reaction was diluted with saturated aqueous sodium bicarbonate and extracted with dichloromethane. The extracts were concentrated and chromatographed (12 g column, 10 to 50% ethyl acetate in hexanes) to give the product as a solid (4 mg). MS: [M+H]⁺ 342.05.

### Compound 154. N-[4-(4-Fluoro-phenyl)-[1,2,3]thiadiazol-5-yl]-2-phenyl-butyramide

4-(4-Fluorophenyl)thiadiazole-5-amine (0.050 g, 0.26 mmol) and triethylamine (0.039 g, 0.39 mmol) were dissolved in anhydrous dichloromethane (2 mL). 2-Phenylbutyryl chloride (0.056 g, 0.31 mmol) was added. The reaction was stirred at room temperature for 3 hours. The reaction was diluted with saturated aqueous sodium bicarbonate and extracted with dichloromethane. The extracts were concentrated and chromatographed (12 g column, 10 to 50% ethyl acetate in hexanes) to give the product as a solid (46 mg). MS: [M+H]⁺ 342.05.

### Compound 155. 2-Phenyl-N-(4-phenyl-[1,2,3]thiadiazol-5-yl)-butyramide

4-Phenylthiadiazole-5-amine (0.045 g, 0.26 mmol) and triethylamine (0.039 g, 0.39 mmol) were dissolved in anhydrous dichloromethane (2 mL). 2-Phenylbutyryl chloride (0.056 g, 0.31 mmol) was added. The reaction was stirred at room temperature for 3 hours. The reaction was diluted with saturated aqueous sodium bicarbonate and extracted with dichloromethane. The extracts were concentrated and chromatographed (12 g column, 10 to 50% ethyl acetate in hexanes) to give the product as a solid (12 mg). MS: [M+H]⁺ 324.06.

### Compound 159. N-(3-(3,4-Difluorobenzylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.1 mmol), (3,4-difluorophenyl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to provide N-(3-(3,4-Difluorobenzylcarbamoyl)thiophen-2-yl)-4-(pyridin-2-yl)piperazine-1-carboxamide (41 mg, 89% yield) as a TFA salt. MS [M+H]⁺=458.

### Compound 160. 2-(2-Phenylbutanamido)-N-(3,4-difluorobenzyl)thiophene-3-carboxamide

2-(2-Phenylbutanamido)thiophene-3-carboxylic acid (29 mg, 0.1 mmol), (3,4-difluorophenyl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to afford 2-(2-phenylbutanamido)-N-(3,4-difluorobenzyl)thiophene-3-carboxamide (37 mg, 89% yield) as a TFA salt. MS [M+H]⁺=415.

### Compound 161. 2-(2-Phenylbutanamido)-N-benzylthiophene-3-carboxamide

2-(2-Phenylbutanamido)thiophene-3-carboxylic acid (29 mg, 0.1 mmol), benzylamine (21 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to produce 2-(2-phenylbutanamido)-N-benzylthiophene-3-carboxamide (28 mg, 75% yield) as a TFA salt. MS [M+H]⁺=379.

### Compound 162. 5-(2-Phenylbutanamido)-N-benzylthiazole-4-carboxamide

5-(2-Phenylbutanamido)thiazole-4-carboxylic acid (29 mg, 0.1 mmol), benzylamine (21 mg, 0.20 mmol), EDCI (38 mg, 0.20 mmol), and HOBt (30 mg, 0.20 mmol) were taken up into 1.0 mL DMF and stirred at room temperature overnight. The reaction was purified by chromatograph to provide 5-(2-phenylbutanamido)-N-benzylthiazole-4-carboxamide (12 mg, 31% yield). MS [M+H]⁺=380.

### Compound 163. 5-(2-Phenylbutanamido)-N-(4-chlorobenzyl)thiazole-4-carboxamide

Ethyl 5-(2-phenylbutanamido)thiazole-4-carboxylate (140 mg, 0.44 mmol) was taken up into 5 mL MeOH/H₂O (1/1) and Lithium hydroxide (184 mg, 4.4 mmol) was added. The reaction stirred at room temperature overnight. The volatiles were removed under vacuum and the solution was acidified with 4 mL 1N HCl. The precipitate was filtered off and 100 mg of product was taken on as is.

5-(2-Phenylbutanamido)thiazole-4-carboxylic acid (29 mg, 0.1 mmol), 4-chlorobenzylamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to produce 5-(2-phenylbutanamido)-N-(4-chlorobenzyl) thiazole-4-carboxamide (13 mg, 31% yield) as a TFA salt. MS [M+H]⁺=414.

### Compound 165. Ethyl 5-(2-phenylbutanamido)thiazole-4-carboxylate

Ethyl 5-aminothiazole-4-carboxylate (250 mg, 1.45 mmol), 2-phenylbutanoyl chloride (265 μL, 1.6 mmol), and pyridine (140 μL, 1.8 mmol) were taken up into 5 mL THF. A catalytic amount of DMAP was added and the reaction stirred overnight at room temperature.

EtOAc was added and then washed with 1N HCl, sat. sodium bicarbonate, and brine. The solvent was removed under vacuum and purified by normal phase chromatography to afford ethyl 5-(2-phenyl butanamido)thiazole-4-carboxylate (140 mg, 30% yield) as a solid. MS [M+H]⁺=319.

### Compound 167. N-(4-(4-Chlorobenzylcarbamoyl)-1,2,3-thiadiazol-5-yl)-1-(pyridin-2-yl)piperidine-4-carboxamide

N-(4-Chlorobenzyl)-5-amino-1,2,3-thiadiazole-4-carboxamide (25 mg, 0.1 mmol), 1-(pyridin-2-yl)piperidine-4-carboxylic acid (21 mg, 0.10 mmol), EDCI (38 mg, 0.20 mmol), and HOBt (30 mg, 0.20 mmol) were taken up into 1.0 mL DMF and stirred at room temperature overnight. The reaction was purified by chromatography to afford N-(4-(4-chlorobenzyl carbamoyl)-1,2,3-thiadiazol-5-yl)-1-(pyridin-2-yl)piperidine-4-carboxamide (8 mg, 18% yield). MS [M+H]⁺=457.

### Compound 168. 4-(2-Fluoro-phenyl)-piperazine-1-carboxylic acid [4-(4-chloro-benzylcarbamoyl)-[1,2,3]thiadiazol-5-yl]-amide

5-Phenoxycarbonylamino-[1,2,3]thiadiazole-4-carboxylic acid ethyl ester (0.177 g, 0.603 mmol), 1-(2-fluorophenyl)piperazine hydrochloride (0.144 g, 0.664 mmol), and diisopropylethylamine (0.117 g, 0.905 mmol) were dissolved in anhydrous tetrahydrofuran (5 mL) and the solution was heated to reflux for 16 hours. The reaction was cooled to room temperature, concentrated to dryness, and chromatographed on silica gel (24 g column, 20 to 50% ethyl acetate in hexanes) to give a white solid, 5-{[4-(2-Fluoro-phenyl)-piperazine-1-carbonyl]-amino}-[1,2,3]thiadiazole-4-carboxylic acid ethyl ester (210 mg).

5-{[4-(2-Fluoro-phenyl)-piperazine-1-carbonyl]-amino}-[1,2,3]thiadiazole-4-carboxylic acid ethyl ester (0.210 g, 0.553 mmol) and lithium hydroxide hydrate (0.116 g, 2.77 mmol) were combined in tetrahydrofuran (20 mL). Water was added until most of the solid went into solution (about 5 mL). The reaction was stirred at room temperature for 3 days. Aqueous hydrochloric acid (0.1 N) was added and the mixture was extracted repeatedly with ethyl acetate. The extracts were concentrated and chromatographed (12 g column, gradient from 100% dichloromethane to 0.5:10:90 acetetic acid:methanol:dichloromethane) to give a white solid, 5-{[4-(2-Fluoro-phenyl)-piperazine-1-carbonyl]-amino}-[1,2,3]thiadiazole-4-carboxylic acid (110 mg).

5-{[4-(2-Fluoro-phenyl)-piperazine-1-carbonyl]-amino}-[1,2,3]thiadiazole-4-carboxylic acid (0.040 g, 0.11 mmol) was dissolved in anhydrous dimethylformamide (2 mL). Diisopropylethylamine (0.022 g, 0.17 mmol) was added followed by benzotriazol-1-yl-oxytripyrrolidinophosphonium hexafluorophosphate (0.071 g, 0.14 mmol). The mixture was stirred for 15 minutes at room temperature and then 4-chlorobenzylamine (0.019 g, 0.14 mmol) was added. The mixture was stirred for 16 hours at room temperature, diluted with brine, and extracted with ethyl acetate. The extracts were concentrated and chromatographed (12 g column, 0 to 40% ethyl acetate in hexanes) to give 4-(2-fluoro-phenyl)-piperazine-1-carboxylic acid [4-(4-chloro-benzylcarbamoyl)-[1,2,3]thiadiazol-5-yl]-amide as a white solid (13 mg). MS: [M+H]⁺ 475.12.

### Compound 169. 4-(2-Fluoro-phenyl)-piperazine-1-carboxylic acid (4-benzylcarbamoyl-[1,2,3]thiadiazol-5-yl)-amide

5-{[4-(2-Fluoro-phenyl)-piperazine-1-carbonyl]-amino}-[1,2,3]thiadiazole-4-carboxylic acid (0.040 g, 0.11 mmol) was dissolved in anhydrous dimethylformamide (2 mL). Diisopropylethylamine (0.022 g, 0.17 mmol) was added followed by benzotriazol-1-yl-oxytripyrrolidinophosphonium hexafluorophosphate (0.071 g, 0.14 mmol). The mixture was stirred for 15 minutes at room temperature and then benzylamine (0.015 g, 0.14 mmol) was added. The mixture was stirred for 16 hours at room temperature, diluted with brine, and extracted with ethyl acetate. The extracts were concentrated and chromatographed (12 g column, 0 to 40% ethyl acetate in hexanes) to give 4-(2-fluoro-phenyl)-piperazine-1-carboxylic acid (4-benzylcarbamoyl-[1,2,3]thiadiazol-5-yl)-amide as a white solid (3 mg). MS: [M+H]⁺ 440.94.

### Compound 170. N-(3-((6-Chloropyridin-3-yl)methylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.1 mmol), (6-chloropyridin-3-yl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC, from which 12 mg (21% yield) of the TFA salt of N-(3-((6-chloropyridin-3-yl)methylcarbamoyl)thiophen-2-yl)-4-(pyridin-2-yl)piperazine-1-carboxamide was produced. MS [M+H]⁺=457.

### Compound 172. N-(3-(N-(2-Methoxybenzyl)-N-methylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.1 mmol), (2-methoxyphenyl)-N-methylmethanamine (30 mg, 0.20 mmol), EDCI (38 mg, 0.20 mmol), and HOBt (30 mg, 0.20 mmol) were taken up into 1.0 mL DMF and stirred at room temperature overnight. The reaction was purified by chromatography, to give N-(3-(N-(2-methoxybenzyl)-N-methylcarbamoyl)thiophen-2-yl)-4-(pyridin-2-yl)piperazine-1-carboxamide (40 mg, 69% yield) as the TFA salt. MS [M+H]⁺=466.

### Compound 173. N-(3-(N-Benzyl-N-methylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.1 mmol), N-methyl(phenyl)methanamine (24 mg, 0.20 mmol), EDCI (38 mg, 0.20 mmol), and HOBt (30 mg, 0.20 mmol) were taken up into 1.0 mL DMF and stirred at room temperature overnight. The reaction was purified by chromatography to give N-(3-(N-Benzyl-N-methylcarbamoyl)thiophen-2-yl)-4-(pyridin-2-yl)piperazine-1-carboxamide (35 mg, 64% yield) as the TFA salt. MS [M+H]⁺=436.

### Compound 174. Methyl 5-(2-(4-fluorophenyl)butanoylcarbamoyl)-2-amino-4-methylthiophene-3-carboxylate

During the preparation of compound 99, a minor byproduct was observed and purified by normal phase chromatograph, and identified as methyl 5-(2-(4-fluorophenyl)butanoylcarbamoyl)-2-amino-4-methylthiophene-3-carboxylate. MS [M+H]⁺=379.

### Compound 175. 5-(4-(Pyridin-2-yl)butanamido)-N-(4-chlorobenzyl)-1,2,3-thiadiazole-4-carboxamide

N-(4-Chlorobenzyl)-5-amino-1,2,3-thiadiazole-4-carboxamide. TFA (40 mg, 0.1 mmol), 4-(pyridin-2-yl)butanoic acid (21 mg, 0.13 mmol), EDCI (29 mg, 0.15 mmol), HOBt (23 mg, 0.15 mmol), and DIEA (52 μL, 0.3 mmol) were taken up into 1.0 mL DMF and stirred at room temperature overnight. The reaction was purified o by preparative HPLC to give 5-(4-(pyridin-2-yl)butanamido)-N-(4-chlorobenzyl)-1,2,3-thiadiazole-4-carboxamide (5 mg, 12% yield) as a TFA salt. MS [M+H]⁺=416.

### Compound 179. 2-(2-Phenylbutanamido)-N-(4-fluorobenzyl)thiophene-3-carboxamide

2-(2-Phenylbutanamido)thiophene-3-carboxylic acid (29 mg, 0.10 mmol), 4-fluorobenzylamine (17 μL, 0.15 mmol), EDCI (29 mg, 0.15 mmol, and HOBt (23 mg, 0.15 mmol) were taken up into 1.0 mL DMF and stirred at room temperature overnight. The reaction was purified by preparative HPLC to give 2-(2-phenylbutanamido)-N-(4-fluorobenzyl) thiophene-3-carboxamide (37 mg, 93% yield) as a TFA salt. MS [M+H]⁺=397.

### Compound 180. N-(4-(4-Fluorobenzylcarbamoyl)-1,2,3-thiadiazol-5-yl)-4-(pyridin-2-yl)piperazine-1-carboxamide

5-(4-(Pyridin-2-yl)piperazine-1-carboxamido)-1,2,3-thiadiazole-4-carboxylic acid (33 mg, 0.10 mmol), 4-fluorobenzylamine (17 μL, 0.15 mmol), EDCI (29 mg, 0.15 mmol, and HOBt (23 mg, 0.15 mmol) were taken up into 1.0 mL DMF and stirred at room temperature overnight. The reaction was purified by preparative HPLC to yield N-(4-(4-fluorobenzylcarbamoyl)-1,2,3-thiadiazol-5-yl)-4-(pyridin-2-yl)piperazine-1-carboxamide (40 mg, 70% yield) as the TFA salt. MS [M+H]⁺=442.

### Compound 181. N-(3-(4-Fluorobenzylcarbamoyl)thiophen-2-yl)-4-(pyridin-2-yl)piperazine-1-carboxamide

2-(4-(Pyridin-2-yl)piperazine-1-carboxamido)thiophene-3-carboxylic acid (33 mg, 0.1 mmol), (4-fluorophenyl)methanamine (25 mg, 0.20 mmol), EDCI (38 mg, 0.20 mmol), and HOBt (30 mg, 0.20 mmol) were taken up into 1.0 mL DMF and stirred at room temperature overnight. The reaction was purified on the prep HPLC. 25 mg (45% yield) of the TFA salt was produced. MS [M+H]⁺=440.

### Compound 182. 1-(4-(4-Fluorobenzylcarbamoyl)-1,2,3-thiadiazol-5-yl)-3-(2-morpholinoethyl)urea

5-(3-(2-Morpholinoethyl)ureido)-1,2,3-thiadiazole-4-carboxylic acid (23 mg, 0.08 mmol), (4-fluorophenyl)methanamine (25 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to yield 1-(4-(4-fluorobenzyl carbamoyl)-1,2,3-thiadiazol-5-yl)-3-(2-morpholinoethyl)urea (21 mg, 50% yield) as the TFA salt. MS [M+H]⁺=409.

### Compound 183. 1-(4-(4-Chlorobenzylcarbamoyl)-1,2,3-thiadiazol-5-yl)-3-(2-morpholinoethyl)urea

Phenyl 4-(ethoxycarbonyl)-1,2,3-thiadiazol-5-ylcarbamate (100 mg, 0.34 mmol) and 2-morpholinoethanamine (66 mg, 0.51 mmol) were taken up into 1.5 mL DMF. DIEA (118 μL, 0.68 mol) was added and the reaction was stirred at room temperature overnight. EtOAc was added and washed with sat. NaHCO₃ and brine. The crude material was purified by normal phase chromatography (0-10% MeOH/DCM). 80 mg (72% yield) of the solid was produced.

Ethyl 5-(3-(2-morpholinoethyl)ureido)-1,2,3-thiadiazole-4-carboxylate (75 mg, 0.23 mmol) was taken up into 2 mL MeOH/H₂O (1/1) and Lithium hydroxide (100 mg, 2.4 mmol) was added. The reaction stirred at room temperature overnight. The volatiles were removed under vacuum and the solution was acidified with 4 mL 1N HCl. The precipitate was filtered off and 46 mg of 5-(3-(2-morpholinoethyl)ureido)-1,2,3-thiadiazole-4-carboxylic acid was obtained.

5-(3-(2-Morpholinoethyl)ureido)-1,2,3-thiadiazole-4-carboxylic acid (23 mg, 0.08 mmol), (4-chlorophenyl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to yield 1-(4-(4-chlorobenzyl carbamoyl)-1,2,3-thiadiazol-5-yl)-3-(2-morpholinoethyl)urea (28 mg, 65% yield) as the TFA salt. MS [M+H]⁺=425.

### Compound 184. 1-(4-(4-Fluorobenzylcarbamoyl)-1,2,3-thiadiazol-5-yl)-3-(2-(pyridin-2-yl)ethyl)urea

5-(3-(2-(Pyridin-2-yl)ethyl)ureido)-1,2,3-thiadiazole-4-carboxylic acid (30 mg, 0.1 mmol), (4-fluorophenyl)methanamine (25 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to afford 1-(4-(4-fluorobenzyl carbamoyl)-1,2,3-thiadiazol-5-yl)-3-(2-(pyridin-2-yl)ethyl)urea (48 mg, 93% yield) as the TFA salt. MS [M+H]⁺=401.

### Compound 185. 1-(4-(4-Chlorobenzylcarbamoyl)-1,2,3-thiadiazol-5-yl)-3-(2-(pyridin-2-yl)ethyl)urea

Phenyl 4-(ethoxycarbonyl)-1,2,3-thiadiazol-5-ylcarbamate (100 mg, 0.34 mmol) and 2-(pyridin-2-yl)ethanamine (62 mg, 0.51 mmol) were taken up into 1.5 mL DMF. DIEA (118 μL, 0.68 mol) was added and the reaction was stirred at room temperature overnight. EtOAc was added and washed with sat. NaHCO₃ and brine. The crude material was purified by normal phase chromatography (0-10% MeOH/DCM). 85 mg (78% yield) of the solid was produced.

Ethyl 5-(3-(2-(pyridin-2-yl)ethyl)ureido)-1,2,3-thiadiazole-4-carboxylate (77 mg, 0.24 mmol) was taken up into 2 mL MeOH/H₂O (1/1) and lithium hydroxide (100 mg, 2.4 mmol) was added. The reaction stirred at room temperature overnight. The volatiles were removed under vacuum and the solution was acidified with 4 mL 1N HCl. The precipitate was filtered off and 60 mg of product was taken on as is.

5-(3-(2-(pyridin-2-yl)ethyl)ureido)-1,2,3-thiadiazole-4-carboxylic acid (30 mg, 0.1 mmol), (4-chlorophenyl)methanamine (28 mg, 0.20 mmol), EDCI (38 mg, 0.20 mmol), and HOBt (30 mg, 0.20 mmol) were taken up into 1.0 mL DMF and stirred at room temperature overnight. The reaction was purified by preparative HPLC to yield 1-(4-(4-chlorobenzyl carbamoyl)-1,2,3-thiadiazol-5-yl)-3-(2-(pyridin-2-yl)ethyl)urea (44 mg, 83% yield) as the TFA salt. MS [M+H]⁺=417.

### Compound 188. Methyl 5-carbamoyl-4-methyl-2-(2-(p-tolyl)butanamido)thiophene-3-carboxylate

2-(p-tolyl)acetic acid (0.60 g, 4.0 mmol) was taken up into 16 mL of dry THF. The solution was cooled to −78° C. via dry ice/acetone bath. Next, n-butyllithium (3.4 mL, 0.0084 mol, 2.1 eq., 2.5 M in hexanes) hexanes was added dropwise. The solution was stirred for two hours at 0° C. Iodoethane (0.40 mL, 4.8 mmol, 1.2 eq.) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 370 mg (52% yield) of 2-(p-tolyl)butanoic acid as a white solid was produced.

2-(p-tolyl)butanoic acid (370 mg, 2.07 mmol) was taken up into 3.7 mL dry DCM under a N2 atmosphere. Oxalyl chloride (196 μL, 2.28 mmol, 1.1 eq.) was added to the solution along with 1 drop of dry DMF. The reaction stirred at room temperature for one hour. The solvent was removed under vacuum and the material placed under high vacuum for one hour. The acyl chloride was taken up into 4 mL pyridine and methyl 2-amino-5-carbamoyl-4-methylthiophene-3-carboxylate (356 mg, 1.66 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified to afford 196 mg (37% yield) of methyl 5-carbamoyl-4-methyl-2-(2-(p-tolyl)butanamido)thiophene-3-carboxylate as a solid TFA salt. MS [M+H]⁺=375.

### Compound 189. Methyl 5-carbamoyl-2-(2-(2-fluorophenyl)butanamido)-4-methylthiophene-3-carboxylate

2-(2-fluorophenyl)acetic acid (0.62 g, 4.0 mmol) was taken up into 16 mL of dry THF. The solution was cooled to −78° C. via dry ice/acetone bath. Next, n-butyllithium (3.4 mL, 0.0084 mol, 2.1 eq., 2.5 M in hexanes) was added dropwise. The solution was stirred for two hours at 0° C. Iodoethane (0.40 mL, 4.8 mmol, 1.2 eq.) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 2-(2-fluorophenyl)butanoic acid was obtained as a clear oil (260 mg, 36% yield).

2-(2-fluorophenyl)butanoic acid (260 mg, 1.42 mmol) was taken up into 2.6 mL dry DCM under a N₂ atmosphere. Oxalyl chloride (134.7 μL, 1.570 mmol, 1.1 eq.) was added to the solution along with 1 drop of dry DMF. The reaction stirred at room temperature for one hour. The solvent was removed under vacuum and the material placed under high vacuum for one hour. The acyl chloride was taken up into 4 mL pyridine and methyl 2-amino-5-carbamoyl-4-methylthiophene-3-carboxylate (245 mg, 1.14 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified to afford 203 mg (38% yield) of methyl 5-carbamoyl-2-(2-(2-fluorophenyl)butanamido)-4-methylthiophene-3-carboxylate as a solid TFA salt. MS [M+H]⁺=379.

### Compound 190. methyl 5-carbamoyl-4-methyl-2-(2-(4-(trifluoromethyl)phenyl)butanamido)thiophene-3-carboxylate

2-(4-(trifluoromethyl)phenyl)acetic acid (0.50 g, 2.4 mmol) was taken up into 10 mL of dry Toluene. The solution was cooled to −48° C. via dry ice/acetonitrile bath. Next, lithium bis(trimethylsilyl)amide (0.86 g, 5.14 mmol, 2.1 eq.) was added dropwise. The solution was stirred for one hour at −48° C. and iodoethane (0.23 mL, 2.9 mmol, 1.2 eq.) was added slowly and the reaction was stirred at room temperature for 30 mins. The reaction mixture was quenched by HCl (1M) to pH=1 and product was extracted with EtOAc. The combined organic layer was dried (Na₂SO₄) and the solvent was removed under reduced pressure. The crude material was purified using normal phase chromatography (0-5% MeOH/DCM). 296 mg (52% yield) of 2-(4-(trifluoromethyl)phenyl)butanoic acid as a white solid was produced.

2-(4-(trifluoromethyl)phenyl)butanoic acid (80 mg, 0.34 mmol) was taken up into 1 mL dry DCM under a N2 atmosphere. Oxalyl chloride (32 μL, 0.37 mmol, 1.1 eq.) was added to the solution along with 1 drop of dry DMF. The reaction stirred at room temperature for one hour. The solvent was removed under vacuum and the material placed under high vacuum for one hour. The acyl chloride was taken up into 2 mL pyridine and methyl 2-amino-5-carbamoyl-4-methylthiophene-3-carboxylate (59 mg, 0.27 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified to afford 65 mg (44% yield) of methyl 5-carbamoyl-4-methyl-2-(2-(4-(trifluoromethyl)phenyl)butanamido)thiophene-3-carboxylate as a solid TFA salt. MS [M+H]⁺=429.

### Compound 191. Methyl 5-carbamoyl-2-(2-(4-chlorophenyl)butanamido)-4-methylthiophene-3-carboxylate

2-(4-chlorophenyl)acetic acid (0.68 g, 4.0 mmol) was taken up into 16 mL of dry THF. The solution was cooled to −78° C. via dry ice/acetone bath. Next, n-butyllithium (3.4 mL, 0.0084 mol, 2.1 eq., 2.5 M in hexanes) was added dropwise. The solution was stirred for two hours at 0° C. Iodoethane (0.40 mL, 4.8 mmol, 1.2 eq.) was added slowly and the reaction was stirred at room temperature overnight. The reaction was quenched with water and the volatiles were removed under vacuum. 1 N HCl was added to the solution and the product was extracted with diethyl ether twice. The crude material was purified using normal phase chromatography (0-5% MeOH/DCM). 450 mg (63% yield) of 2-(4-chlorophenyl)butanoic acid as a clear oil was produced.

2-(4-chlorophenyl)butanoic acid (450 mg, 2.52 mmol) was taken up into 5 mL dry DCM under a N2 atmosphere. Oxalyl chloride (238 μL, 2.77 mmol, 1.1 eq.) was added to the solution along with 1 drop of dry DMF. The reaction stirred at room temperature for one hour. The solvent was removed under vacuum and the material placed under high vacuum for one hour. The acyl chloride was taken up into 6 mL pyridine and methyl 2-amino-5-carbamoyl-4-methylthiophene-3-carboxylate (432 mg, 2.02 mmol) was added. A catalytic amount of DMAP was added and the reaction stirred at room temperature overnight. EtOAc was added and then washed with 1N HCl, sat. sodium bicarbonate, and brine. The solvent was removed under vacuum and purified to afford 465 mg (35% yield) of methyl 5-carbamoyl-2-(2-(4-chlorophenyl)butanamido)-4-methylthiophene-3-carboxylate as a solid TFA salt. MS [M+H]⁺=394.87.

### Compound 192. 2-tert-butyl 4-methyl 5-(2-(benzo[d][1,3]dioxol-5-yl)butanamido)-3-methylthiophene-2,4-dicarboxylate

2-(benzo[d][1,3]dioxol-6-yl)acetic acid (0.61 g, 3.37 mmol) was taken up into 15 mL of dry THF, the solution was cooled to −78° C. via dry ice/acetone bath, then n-butyllithium (3.0 mL, 7.0 mmol, 2.1 equiv., 2.5 M) in hexanes was added dropwise. The solution was stirred for two hours at 0° C., then iodoethane (0.33 mL, 3.7 mmol, 1.1 equiv.) was added slowly, and the reaction was stirred at room temperature overnight. The reaction was quenched with 5 mL water and the volatiles were removed in vacuo. 1 N HCl was added to the resultant solution and the solution was extracted twice with diethyl ether. The crude material was purified using normal phase chromatography (0-5% MeOH/DCM) to provide the 2-(benzo[d][1,3]dioxol-6-yl)butanoic acid as a white solid (0.38 g, 54% yield).

2-(benzo[d][1,3]dioxol-6-yl)butanoic acid (0.235 g, 1.13 mmol) was taken up into 5 mL dry DCM under a N2 atmosphere. Oxalyl chloride (0.11 mL, 1.24 mmol, 1.1 equiv) was added to the solution along with 2 drops of dry DMF. The reaction stirred at room temperature for one hour. The reaction progress (i.e., formation of the acyl chloride) was assessed by quenching an aliquot of the reaction mixture with MeOH, wherein the reaction was considered complete when only the methyl ester was observed by LC/MS analysis of the treated aliquot. The solvent was removed in vacuo and the material placed under high vacuum for one hour. 2-tert-butyl 4-methyl 5-amino-3-methylthiophene-2,4-dicarboxylate (0.28 g, 1.0 mmol) was added along with 5 mL pyridine. A catalytic amount of DMAP was added and the reaction stirred at room temperature overnight. 50 mL of EtOAc was added and then washed with 1N HCl, sat. sodium bicarbonate, and brine. The solvent was removed in vacuo and the crude material was purified by normal phase chromatography (0-20% EtOAc/DCM) to obtain the title compound as a white solid (0.18 g, 35% yield). MS: [M+H]⁺ 462.

### Compound 193. Methyl 2-(2-(benzo[d][1,3]dioxol-5-yl)butanamido)-5-carbamoyl-4-methylthiophene-3-carboxylate

2-tert-butyl 4-methyl 5-(2-(benzo[d][1,3]dioxol-5-yl)butanamido)-3-methylthiophene-2,4-dicarboxylate (60 mg, 0.13 mmol) was taken up into 0.5 mL DCM and 0.25 mL TFA and stirred for 1 hr. The solvent was removed in vacuo and the material placed under high vacuum for one hour. Next, DMF (1 mL) was added along with ammonium chloride (65 mg, 1.02 mmol), EDCI (37 mg, 0.2 mmol), HOBt (30 mg, 0.2 mmol) and triethylamine (0.17 mL, 1.2 mmol). The reaction was stirred overnight at room temperature. The reaction was quenched with water and extracted with EtOAc. The crude material was purified by preparative HPLC to provide the title compound as a white powder (30 mg, 57% yield). MS: [M+H]⁺ 405.

### Compound 194. 2-tert-butyl 4-methyl 5-(2-(3,5-difluorophenyl)butanamido)-3-methylthiophene-2,4-dicarboxylate

2-(3,5-difluorophenyl)acetic acid (0.655 g, 3.8 mmol) was taken up into 15 mL of dry THF, the solution was cooled to −78° C. via dry ice/acetone bath, then n-butyllithium (3.4 mL, 8.0 mmol, 2.1 equiv. 2.5 M) in hexanes was added dropwise. The solution was stirred for two hours at 0° C., then iodoethane (0.37 mL, 4.2 mmol, 1.1 equiv.) was added slowly, and the reaction was stirred at room temperature overnight. The reaction was quenched with 5 mL water and the volatiles were removed in vacuo. 1 N HCl was added to the resultant solution and the solution was extracted twice with diethyl ether. The crude material was purified using normal phase chromatography (0-5% MeOH/DCM) to provide 2-(3,5-difluorophenyl)butanoic acid as a white solid (0.42 g, 55% yield).

2-(3,5-difluorophenyl)butanoic acid (0.42 g, 2.1 mmol) was taken up into 10 mL dry DCM under a N2 atmosphere. Oxalyl chloride (0.20 mL, 2.3 mmol, 1.1 equiv.) was added to the solution along with 2 drops of dry DMF. The reaction stirred at room temperature for one hour. The reaction progress (i.e., formation of the acyl chloride) was assessed by quenching an aliquot of the reaction mixture with MeOH, wherein the reaction was considered complete when only the methyl ester was observed by LC/MS analysis of the treated aliquot. The solvent was removed in vacuo and the material placed under high vacuum for one hour. 2-tert-butyl 4-methyl 5-amino-3-methylthiophene-2,4-dicarboxylate (0.51 g, 1.9 mmol) was added along with 5 mL pyridine. A catalytic amount of DMAP was added and the reaction stirred at room temperature overnight. EtOAc (150 mL) was added and then washed with 1N HCl, sat. sodium bicarbonate, and brine. The solvent was remove in vacuo and the crude material was purified by normal phase chromatography (0-20% EtOAc/DCM) to provide the title compound as a white solid (0.49 g, 52% yield). MS: [M+H]⁺ 454.

### Compound 195. Methyl 2-(2-(3,5-difluorophenyl)butanamido)-5-carbamoyl-4-methylthiophene-3-carboxylate

2-tert-butyl 4-methyl 5-(2-(3,5-difluorophenyl)butanamido)-3-methylthiophene-2,4-dicarboxylate (60 mg, 0.13 mmol) was taken up into 1.0 mL DCM and 0.50 mL TFA and stirred for 1 hr. The solvent was removed in vacuo and the material placed under high vacuum for one hour. DMF (2 mL) was added along with ammonium chloride (65 mg, 1.02 mmol), EDCI (37 mg, 0.2 mmol), HOBt (30 mg, 0.2 mmol) and triethylamine (0.17 mL, 1.2 mmol). The reaction stirred overnight at room temperature. The reaction was quenched with water and extracted with EtOAc. The crude material was purified by preparative HPLC to provide the title compound as a white powder (10 mg, 20% yield). MS: [M+H]⁺ 397.

### Compound 196. 2-tert-butyl 4-methyl 5-(2-(4-cyanophenyl)butanamido)-3-methylthiophene-2,4-dicarboxylate

2-(4-cyanophenyl)acetic acid (0.745 g, 4.6 mmol) was taken up into 20 mL of dry THF, the solution was cooled to −78° C. via dry ice/acetone bath, then n-butyllithium (4.0 mL, 9.7 mmol, 2.1 equiv., 2.5 M) in hexanes was added dropwise. The solution was stirred for two hours at 0° C., then iodoethane (0.45 mL, 5.5 mmol, 1.1 equiv.) was added slowly, and the reaction was stirred at room temperature overnight. The reaction was quenched with 5 mL water and the volatiles were removed in vacuo. 1 N HCl was added to the resultant solution and the solution was extracted twice with diethyl ether. The crude material was purified using normal phase chromatography (0-5% MeOH/DCM) to provide 2-(4-cyanophenyl)butanoic acid as a white solid (0.41 g, 47% yield).

2-(4-cyanophenyl)butanoic acid (0.41 g, 2.1 mmol) was taken up into 10 mL dry DCM under a N2 atmosphere. Oxalyl chloride (0.20 mL, 2.3 mmol, 1.1 equiv.) was added to the solution along with 2 drops of dry DMF. The reaction progress (i.e., formation of the acyl chloride) was assessed by quenching an aliquot of the reaction mixture with MeOH, wherein the reaction was considered complete when only the methyl ester was observed by LC/MS analysis of the treated aliquot. The solvent was removed in vacuo and the material placed under high vacuum for one hour. 2-tert-butyl 4-methyl 5-amino-3-methylthiophene-2,4-dicarboxylate (0.51 g, 1.9 mmol) was added along with 5 mL pyridine. A catalytic amount of DMAP was added and the reaction stirred at room temperature overnight. EtOAc (150 mL) was added and then washed with 1N HCl, sat. sodium bicarbonate, and brine. The solvent was remove in vacuo and purified by normal phase chromatography (0-20% EtOAc/DCM) to provide the title compound as a white solid (0.35 g, 38% yield). MS: [M+H]⁺ 443.

### Compound 197. Methyl 2-(2-(4-cyanophenyl)butanamido)-5-carbamoyl-4-methylthiophene-3-carboxylate

2-tert-butyl 4-methyl 5-(2-(4-cyanophenyl)butanamido)-3-methylthiophene-2,4-dicarboxylate (66 mg, 0.15 mmol) was taken up into 1.0 mL DCM and 0.50 mL TFA, and stirred for 1 hr. The solvent was removed in vacuo and the material placed under high vacuum for one hour. 2 mL of DMF was added along with ammonium chloride (65 mg, 1.2 mmol), EDCI (37 mg, 0.2 mmol), HOBt (30 mg, 0.2 mmol) and triethylamine (0.17 mL, 1.2 mmol). The reaction stirred overnight at room temperature. The reaction was quenched with water and extracted with EtOAc. The crude material was purified on the preparative HPLC to provide the title compound as a white powder (42 mg, 73% yield). MS: [M+H]⁺ 386.

### Compound 198. 2-tert-butyl 4-methyl 5-(2-(4-(trifluoromethyl)phenyl)butanamido)-3-methylthiophene-2,4-dicarboxylate

2-(4-(trifluoromethyl)phenyl)butanoic acid (0.965 g, 4.16 mmol) was taken up into 50 mL anhydrous DCM under a nitrogen atmosphere. Oxalyl chloride (2.0 M in DCM, 2.08 mL, 4.16 mmol) was added to the solution along with 2 drops of anhydrous DMF. The reaction stirred at room temperature for one hour. The solvent was evaporated and 2-tert-butyl 4-methyl-5-amino-3-methylthiophene-2,4-dicarboxylate (0.51 g, 1.9 mmol) was added along with 10 mL pyridine. 4-Dimethylaminopyridine (51 mg, 0.42 mmol) was added and the reaction stirred at room temperature overnight. The mixture was diluted with brine and extracted with ethyl acetate. The extracts were washed with 1 N HCl and then concentrated. The resulting solid was chromatographed (40 g silica column, EtOAc/hexanes) to provide the title compound as an of-white solid (0.61 g, 66% yield). MS: [M+H]⁺ 486.2.

### Compound 199. 4-(methoxycarbonyl)-5-(2-(4-(trifluoromethyl)phenyl)butanamido)-3-methylthiophene-2-carboxylic acid

2-tert-butyl 4-methyl 5-(2-(4-(trifluoromethyl)phenyl)butanamido)-3-methylthiophene-2,4-dicarboxylate (0.60 g, 1.25 mmol) was dissolved in DCM (18 mL) and then trifluoroacetic acid (2 mL) was added. The solution was stirred at room temperature overnight and then concentrated to provide the title compound as an off-white solid (0.53 g, quantitative yield). MS: [M+H]⁺ 430.1.

### Compound 200. Methyl 5-(2-aminoethylcarbamoyl)-2-(2-(4-(trifluoromethyl)phenyl)butanamido)-4-methylthiophene-3-carboxylate

4-(methoxycarbonyl)-5-(2-(4-(trifluoromethyl)phenyl)butanamido)-3-methylthiophene-2-carboxylic acid (0.384 g, 0.90 mmol) was dissolved in anhydrous dichloromethane (10 mL). Oxalyl chloride (2M in dichloromethane, 0.450 mL) and dimethylformamide (˜5 drops) were added. The reaction was stirred at room temperature for 1 hour. Next, tert-butyl 2-aminoethylcarbamate (0.173 g, 1.08 mmol) and diisopropylethylamine (0.289 g, 2.24 mmol) were added and the reaction was stirred at room temperature overnight. The reaction was diluted with saturated aqueous sodium bicarbonate and extracted with dichloromethane. The extracts were concentrated and chromatographed (12 g silica column, hexanes/ethyl acetate). The resulting product was dissolved in a solution of HCl/dioxane (4 N, 20 mL). After two hours, the solution was lyophilized to provide the hydrochloride salt of the title compound as an off-white powder (0.305 g, 67% yield). MS: [M+H]⁺ 472.4

### Compound 201. Methyl 2-[2-(4-fluorophenyl)butanamido]-4-methyl-5-(piperazine-1-carbonyl)thiophene-3-carboxylate

4-(methoxycarbonyl)-5-(2-(4-fluorophenyl)butanamido)-3-methylthiophene-2-carboxylic acid (0.18 g, 0.47 mmol) was dissolved in anhydrous dichloromethane (10 mL). Oxalyl chloride (2M in dichloromethane, 0.237 mL) and dimethylformamide (˜5 drops) were added. The reaction was stirred at room temperature for 1 hour. Piperazine (0.122 g, 1.41 mmol) was added and the reaction was stirred at room temperature overnight. The reaction was diluted with brine and extracted with ethyl acetate. The extract was diluted with saturated aqueous sodium bicarbonate and extracted with dichloromethane. The extracts were concentrated and chromatographed (12 g silica column, hexanes/ethyl acetate). The resulting product was dissolved in a solution of HCl/dioxane (4 N, 3 mL) and lyophilized to provide the hydrochloride salt of the title compound as an off-white powder (0.10 g, 44% yield). MS: [M+H]⁺ 448.2.

### Compound 202. Methyl 5-((3-((tert-butoxycarbonyl)amino)propyl)carbamoyl)-2-(2-(4-fluorophenyl)butanamido)-4-methylthiophene-3-carboxylate

4-(Methoxycarbonyl)-5-(2-(4-fluorophenyl)butanamido)-3-methylthiophene-2-carboxylic acid (0.090 g, 0.24 mmol) was dissolved in anhydrous dimethylformamide (2 mL), then PyBOP (0.146 g, 0.28 mmol) was added. The reaction was stirred at room temperature for 15 minutes. Next, diisopropylethylamine (0.077 g, 0.60 mmol) and tert-butyl 3-aminopropylcarbamate (0.050 g, 0.28 mmol) were added and the reaction was stirred at room temperature overnight. The reaction was diluted with brine and extracted with ethyl acetate. The extracts were concentrated and chromatographed (12 g silica column; hexanes/ethyl acetate) to provide the title compound as a white solid (0.085 g, 66% yield). MS: [M+H]⁺ 536.5.

### Compound 203. Methyl 5-(3-aminopropylcarbamoyl)-2-(2-(4-fluorophenyl)butanamido)-4-methylthiophene-3-carboxylate

Tert-butyl 3-(methyl 2-(2-(4-fluorophenyl)butanamido)-5-carbamoyl-4-methylthiophene-3-carboxyloyl)propylcarbamate (0.080 g, 0.15 mmol) was dissolved in a solution of hydrogen chloride in dioxane (4 N, 2 mL). After two hours the solution was lyophilized to provide the hydrochloride salt of the title compound as a white powder (0.073 g, quantitative yield). MS: [M+H]⁺ 436.5.

### Example 5: Biological Assays

### IC₅₀ In Vitro DNA Synthesis Assay: Processive DNA Synthesis Assay

The Rapid Plate Assay was performed as previously described (Lin & Ricciardi, 2000, J. Virol. Methods 88:219-225). Briefly, a 5′-biotinylated 100-nucleotide template that contains adenines only at its 5′-distal end was annealed with a 15-nucleotide primer to its 3′-end and attached to streptavidin-coated 96-plate wells (Roche Applied Science). DNA synthesis was carried out in 50 μL reaction mixture containing 100 mM (NH)₂SO₄, 20 mM Tris-HCl (pH 7.5), 3 mM MgCl₂, 0.1 mM EDTA, 0.5 mM DTT, 2% glycerol, 40 μg/ml BSA, 5 μM dATP, 5 M dCTP, 5 M dGTP, 1 M digoxigenin-11-dUTP, and E9/A20/D4 proteins. The TNT reticulocyte lysate containing vaccinia virus vD4 or molluscum mD4 and vEC50 and vA20 or in vitro translated luciferase was used as a negative control were added to the reaction mixture. After incubation at 37° C. for 30 min, the plate was washed extensively with phosphate-buffered saline (PBS). The wells were then incubated with anti-digoxigenin-peroxidase antibody (Roche) for 1 h at 37° C., followed by washing with PBS. The substrate 2,2′-azino-bis(3-ethylbenzthiazoline)-sulfonate (Roche) was added, and plates were gently rocked to allow color development. DNA synthesis was quantified by measuring the absorbance of each reaction at 405 nm with a microplate reader (Tecan). Experiments were conducted in triplicate and independently repeated at least twice. Increasing concentrations of compounds of interest were independently added to the total reaction mixture to obtain IC₅₀ values.

### EC₅₀ Plaque Reduction Assay: Viral Plaque Reduction

Viral plaque reduction assay was performed using BSC-1 cells as previously described (Nuth, et al., 2011, J. Med. Chem. 54:3260-3267) in triplicate, and independently repeated with compounds of interest. Cells were infected by adsorbing poxvirus at 80 PFU/well in 100 μL of growth medium for 1 h in 48-well plate, followed by 16 h treatment with compounds. Cells were stained and plaques counted and data was plotted on GraphPad Prism.

TABLE 1
Summary of DSF experiments at 25
and 50 μM compound treatments

| | ΔTₘ (° C.) |
|---|---|
| Compound | 25 μM, 50 μM |
| 1 | −0.86 ± 0.43; |
| | −1.55 ± 0.16 |
| CDV | −0.37 ± 0.14; |
| | −0.27 ± 0.37 |
| ST-246 | −0.37 ± 0.08; |
| | −0.26 ± 0.10 |

Tₘ = 42.24 ± 0.66° C. for D4. Values for compound 10 reflect n = 9 and n = 3 for cidofovir (CDV) and tecovirimat (ST-246).

TABLE 2
Compounds and biological activity

| | | | VV | VV | mD4 | mD4 |
|---|---|---|---|---|---|---|
| | | | EC₅₀ | IC₅₀ | EC₅₀ | IC₅₀ |
| Cmpd | Structure | Formula | (μM) | (μM) | (μM) | (μM) |
| 1 | 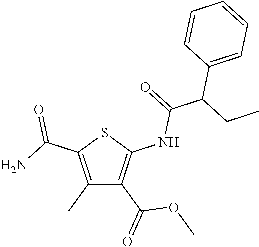 [SMILES: CCC(C(=O)Nc1sc(C(N)=O)c(C)c1C(=O)OC)c1ccccc1] | C₁₈H₂₀N₂O₄S | 15.1 | 16 | 22.9 | 16.3 |
| | methyl 5-carbamoyl-4-methyl-2-(2- | | | | | |
| | phenylbutanamido)thiophene-3- | | | | | |
| | carboxylate | | | | | |
| 2 | 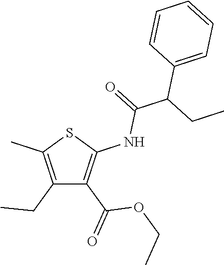 [SMILES: CCOC(=O)c1c(NC(=O)C(CC)c2ccccc2)sc(C)c1CC] | C₂₀H₂₅NO₃S | 36.6 | ~100 | | |
| | ethyl 4-ethyl-5-methyl-2-(2- | | | | | |
| | phenylbutanamido)thiophene-3- | | | | | |
| | carboxylate | | | | | |
| 4 | 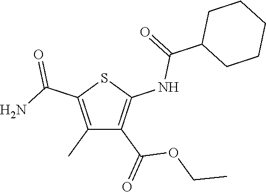 [SMILES: CCOC(=O)c1c(NC(=O)C2CCCCC2)sc(C(N)=O)c1C] | C₁₆H₂₂N₂O₄S | 48.9 | ~100 | | |
| | ethyl 5-carbamoyl-2- | | | | | |
| | (cyclohexanecarboxamido)-4- | | | | | |
| | methylthiophene-3-carboxylate | | | | | |
| 5 | 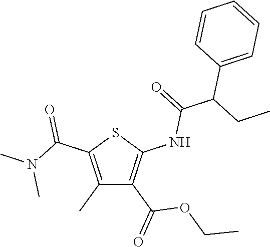 [SMILES: CCOC(=O)c1c(NC(=O)C(CC)c2ccccc2)sc(C(=O)N(C)C)c1C] | C₂₀H₂₄N₂O₄S | 29.7 | ~100 | | |
| | ethyl 5-(dimethylcarbamoyl)-4- | | | | | |
| | methyl -2-(2- | | | | | |
| | phenylbutanamido)thiophene-3- | | | | | |
| | carboxylate | | | | | |
| 8 | 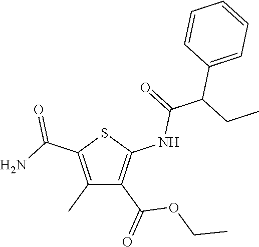 [SMILES: CCOC(=O)c1c(NC(=O)C(CC)c2ccccc2)sc(C(N)=O)c1C] | C₁₉H₂₂N₂O₄S | 10.3 | 54.6 | | |
| | ethyl 5-carbamoyl-4-methyl-2-(2- | | | | | |
| | phenylbutanamido)thiophene-3- | | | | | |
| | carboxylate | | | | | |
| 9 | 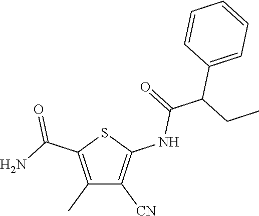 [SMILES: CCC(C(=O)Nc1sc(C(N)=O)c(C)c1C#N)c1ccccc1] | C₁₇H₁₇N₃O₂S | 11.3 | >200 | | |
| | 4-cyano-3-methyl-5-(2- | | | | | |
| | phenylbutanamido)thiophene-2- | | | | | |
| | carboxamide | | | | | |
| 15 | 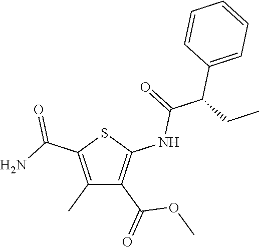 [SMILES: CC[C@H](C(=O)Nc1sc(C(N)=O)c(C)c1C(=O)OC)c1ccccc1] | C₁₈H₂₀N₂O₄S | 17.9 | 30.4 | | |
| | methyl (S)-5-carbamoyl-4-methyl- | | | | | |
| | 2-(2-phenylbutanamido)thiophene- | | | | | |
| | 3-carboxylate | | | | | |
| 16 | 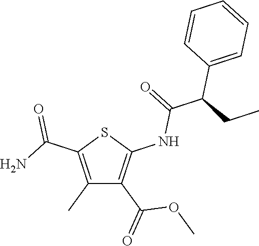 [SMILES: CC[C@@H](C(=O)Nc1sc(C(N)=O)c(C)c1C(=O)OC)c1ccccc1] | C₁₈H₂₀N₂O₄S | 16.4 | | | |
| | methyl (R)-5-carbamoyl-4-methyl- | | | | | |
| | 2-(2-phenylbutanamido)thiophene- | | | | | |
| | 3-carboxylate | | | | | |
| 17 | 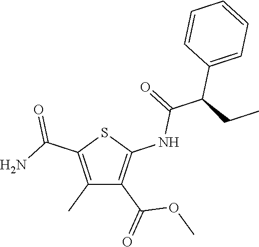 [SMILES: CC[C@@H](C(=O)Nc1sc(C(N)=O)c(C)c1C(=O)OC)c1ccccc1] | C₂₂H₂₀N₂O₄S | 26.8 | 136.5 | | |
| | methyl (R)-5-carbamoyl-4-methyl- | | | | | |
| | 2-(2-phenylbutanamido)thiophene- | | | | | |
| | 3-carboxylate | | | | | |
| 19 | 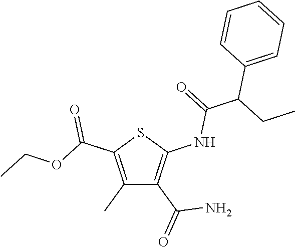 [SMILES: CCOC(=O)c1sc(NC(=O)C(CC)c2ccccc2)c(C(N)=O)c1C] | C₁₉H₂₂N₂O₄S | 31.3 | 178.5 | | |
| | ethyl 4-carbamoyl-3-methyl-5-(2- | | | | | |
| | phenylbutanamido)thiophene-2- | | | | | |
| | carboxylate | | | | | |
| 22 | 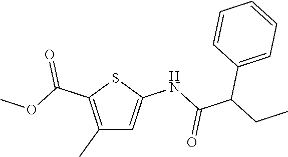 [SMILES: CCC(C(=O)Nc1cc(C)c(C(=O)OC)s1)c1ccccc1] | C₁₇H₁₉NO3S | 26.8 | | | |
| | 3-Methyl-5-(2-phenyl- | | | | | |
| | butyrylamino)-thiophene-2- | | | | | |
| | carboxylic acid methyl ester | | | | | |
| 24 | 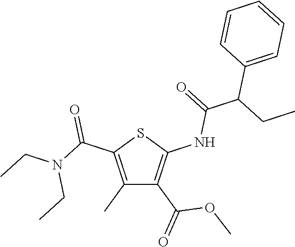 [SMILES: CCC(C(=O)Nc1sc(C(=O)N(CC)CC)c(C)c1C(=O)OC)c1ccccc1] | C₂₂H₂₈N₂O₄S | 16.4 | 188 | | |
| | methyl 5-(diethylcarbamoyl)-4- | | | | | |
| | methyl-2-(2- | | | | | |
| | phenylbutanamido)thiophene-3- | | | | | |
| | carboxylate | | | | | |
| 26 | 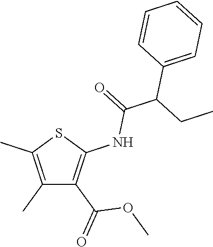 [SMILES: CCC(C(=O)Nc1sc(C)c(C)c1C(=O)OC)c1ccccc1] | C₁₈H₂₁NO₃S | 45.3 | | | |
| | methyl 4,5-dimethyl-2-(2- | | | | | |
| | phenylbutanamido)thiophene-3- | | | | | |
| | carboxylate | | | | | |
| 29 | 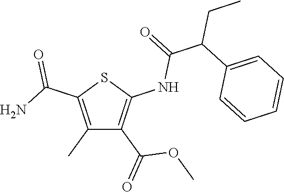 [SMILES: CCC(C(=O)Nc1sc(C(N)=O)c(C)c1C(=O)OC)c1ccccc1] | C₁₉H₂₂N₂O₄S | 28 | 106.5 | 19.7 | 13.4 |
| | Methyl 2-(2-phenylbutanamido)-5- | | | | | |
| | carbamoyl-4-methylthiophene-3- | | | | | |
| | carboxylate | | | | | |
| 31 | 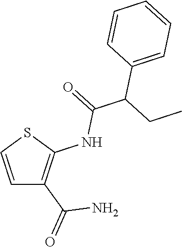 [SMILES: CCC(C(=O)Nc1sccc1C(N)=O)c1ccccc1] | C₁₅H₁₆N₂O₂S | 49.7 | | | |
| | 2-(2-phenylbutanamido)thiophene- | | | | | |
| | 3-carboxamide | | | | | |
| 32 | 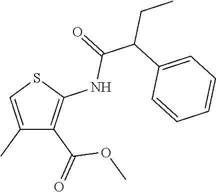 [SMILES: CCC(C(=O)Nc1scc(C)c1C(=O)OC)c1ccccc1] | C₁₇H₁₉NO₃S | 32.9 | >200 | | |
| | 4-Methyl-2-(2-phenyl- | | | | | |
| | butyrylamio)-thiophene-3- | | | | | |
| | carboxylic acid methyl ester | | | | | |
| 33 | 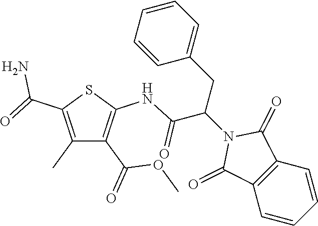 [SMILES: COC(=O)c1c(NC(=O)C(Cc2ccccc2)N2C(=O)c3ccccc3C2=O)sc(C(N)=O)c1C] | C₂₅H₂₁N₃O₆S | 43.6 | 136.4 | | |
| | 5-Carbamoyl-2-[2-(1,3-dioxo-1,3- | | | | | |
| | dihydro-isoindol-2-yl)-3-phenyl- | | | | | |
| | propionylamino]-4-methyl- | | | | | |
| | thiophene-3-carboxylic acid methyl | | | | | |
| | ester | | | | | |
| 34 | 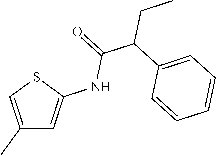 [SMILES: CCC(C(=O)Nc1cc(C)cs1)c1ccccc1] | C₁₅H₁₇NOS | 46.7 | | | |
| | N-(4-methyl-thiophen-2-yl)-2- | | | | | |
| | phenyl-butyramide | | | | | |
| 35 | 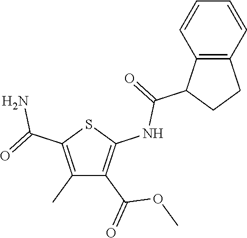 [SMILES: COC(=O)c1c(NC(=O)C2CCc3ccccc32)sc(C(N)=O)c1C] | C₁₈H₁₈N₂O₄S | 20.4 | 128.7 | | |
| | 5-Carbamoyl-2-[(indane-1- | | | | | |
| | carbonyl)-amino]-4-methyl- | | | | | |
| | thiophene-3-carboxylic acid methyl | | | | | |
| | ester | | | | | |
| 36 | 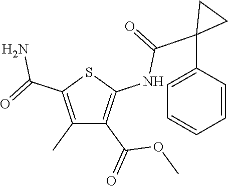 [SMILES: COC(=O)c1c(NC(=O)C2(c3ccccc3)CC2)sc(C(N)=O)c1C] | C₁₈H₁₈N₂O4S | 14.3 | 128.5 | | |
| | 5-Carbamoyl-4-methyl-2-[(1- | | | | | |
| | phenyl-cyclopropanecarbonyl)- | | | | | |
| | amino]-thiophene-3-carboxylic acid | | | | | |
| | methyl ester | | | | | |
| 39 | 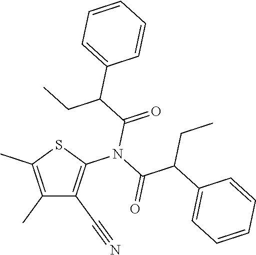 [SMILES: CCC(C(=O)N(C(=O)C(CC)c1ccccc1)c1sc(C)c(C)c1C#N)c1ccccc1] | C₂₇H₂₈N₂O₂S | 11 | >200 | | |
| | N-(3-Cyano-4,5-dimethyl-thiophen- | | | | | |
| | 2-yl)-2-phenyl-N-(2-phenyl- | | | | | |
| | butyryl)-butyramide | | | | | |
| 40 | 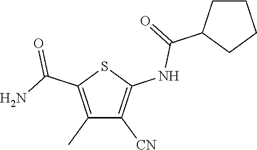 [SMILES: Cc1c(C(N)=O)sc(NC(=O)C2CCCC2)c1C#N] | C₁₃H₁₅N₃O₂S | 47.4 | >200 | | |
| | 4-cyano-5- | | | | | |
| | (cyclopentanecarboxamido)-3- | | | | | |
| | methylthiophene-2-carboxamide | | | | | |
| 47 | 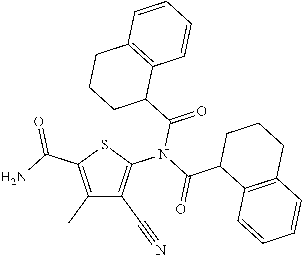 [SMILES: Cc1c(C(N)=O)sc(N(C(=O)C2CCCc3ccccc32)C(=O)C2CCCc3ccccc32)c1C#N] | C₂₉H₂₇N₃O₃S | 20 | 47- 140 | | |
| | 5-[Bis-(1,2,3,4-tetrahydro- | | | | | |
| | naphthalene-1-carbonyl)-amino]-4- | | | | | |
| | cyano-3-methyl-thiophene-2- | | | | | |
| | carboxylic acid amide | | | | | |
| 50 | 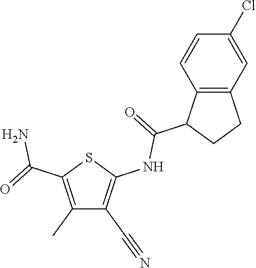 [SMILES: Cc1c(C(N)=O)sc(NC(=O)C2CCc3cc(Cl)ccc32)c1C#N] | C₁₇H₁₄ClN₃O₂S | 33 | | | |
| | 5-[(5-Chloro-indane-1-carbonyl)- | | | | | |
| | amino]-4-cyano-3-methyl- | | | | | |
| | thiophene-2-carboxylic acid amide | | | | | |
| 57 | 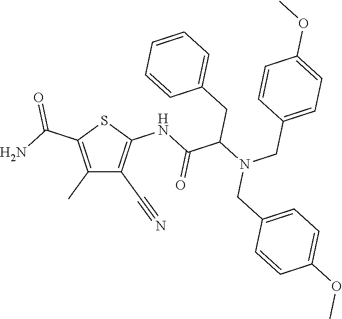 [SMILES: COc1ccc(CN(Cc2ccc(OC)cc2)C(Cc2ccccc2)C(=O)Nc2sc(C(N)=O)c(C)c2C#N)cc1] | C₃₂H₃₂N₄O₄S | 20.8 | 63 | | |
| | 5-(2-{Bis[(4- | | | | | |
| | methoxyphenyl)methyl]amino}-3- | | | | | |
| | phenylpropanamido)-4-cyano-3- | | | | | |
| | methylthiophene-2-carboxamide | | | | | |
| 66 | 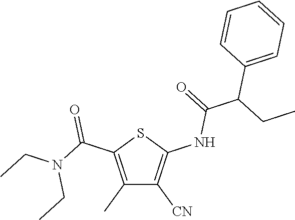 [SMILES: CCC(C(=O)Nc1sc(C(=O)N(CC)CC)c(C)c1C#N)c1ccccc1] | C₂₁H₂₅N₃O₂S | 42.2 | 177 | | |
| | 4-cyano-N,N-diethyl-3-methyl-5- | | | | | |
| | (2-phenylbutanamido)thiophene-2- | | | | | |
| | carboxamide | | | | | |
| 68 | 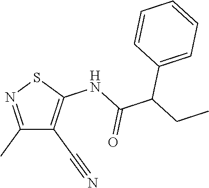 [SMILES: CCC(C(=O)Nc1snc(C)c1C#N)c1ccccc1] | C₁₅H₁₅N₃OS | 8.6 | | | |
| | N-(4-Cyano-3-methyl-isothiazol-5- | | | | | |
| | yl)-2-phenyl-butyramide | | | | | |
| 72 | 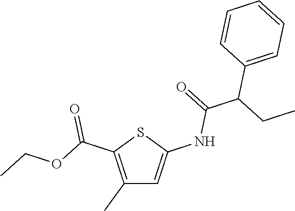 [SMILES: CCOC(=O)c1sc(NC(=O)C(CC)c2ccccc2)cc1C] | C₁₈H₂₁NO₃S | 19.1 | | | |
| | ethyl 3-methyl-5-(2- | | | | | |
| | phenylbutanamido)thiophene-2- | | | | | |
| | carboxylate | | | | | |
| 75 | 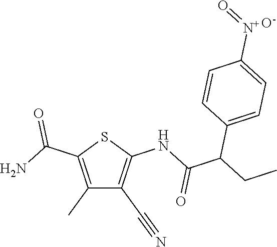 | C₁₇H₁₆N₄O₄S | 35 | | | |
| | 5-(2-(4-Nitrophenyl)butanamido)- | | | | | |
| | 4-cyano-3-methylthiophene-2- | | | | | |
| | carboxamide | | | | | |
| 76 | 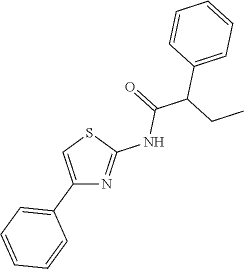 [SMILES: CCC(C(=O)Nc1nc(-c2ccccc2)cs1)c1ccccc1] | C₁₉H₁₈N₂OS | 18.4 | | | |
| | 2-phenyl-N-(4-phenylthiazol-2- | | | | | |
| | yl)butanamide | | | | | |
| 77 | 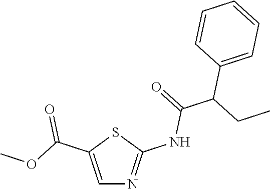 [SMILES: CCC(C(=O)Nc1ncc(C(=O)OC)s1)c1ccccc1] | C₁₅H₁₆N₂O₃S | 21.4 | | | |
| | methyl 2-(2- | | | | | |
| | phenylbutanamido)thiazole-5- | | | | | |
| | carboxylate | | | | | |
| 78 | 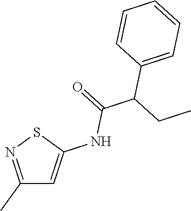 [SMILES: CCC(C(=O)Nc1cc(C)ns1)c1ccccc1] | C₁₄H₁₆N₂OS | 41.7 | | | |
| | N-(3-methylisothiazol-5-yl)-2- | | | | | |
| | phenylbutanamide | | | | | |
| 82 | 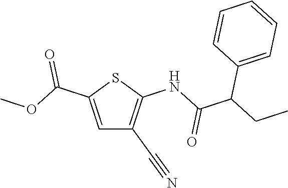 [SMILES: CCC(C(=O)Nc1sc(C(=O)OC)cc1C#N)c1ccccc1] | C₁₇H₁₆N₂O₃S | 11 | | | |
| | Methyl 5-(2-phenylbutanamido)-4- | | | | | |
| | cyanothiophene-2-carboxylate | | | | | |
| 83 | 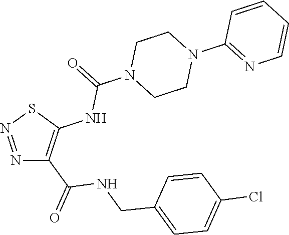 [SMILES: O=C(NCc1ccc(Cl)cc1)c1nnsc1NC(=O)N1CCN(c2ccccn2)CC1] | C₂₀H₂₀ClN₇O₂S | 7.3 | 20 | 7.7 | 31.4 |
| | N-(4-chlorobenzyl)-5-(4-(pyridin- | | | | | |
| | 2-yl)piperazine-1-carboxamido)- | | | | | |
| | 1,2,3-thiadiazole-4-carboxamide | | | | | |
| 84 | 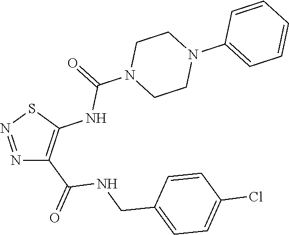 [SMILES: O=C(NCc1ccc(Cl)cc1)c1nnsc1NC(=O)N1CCN(c2ccccc2)CC1] | C₂₁H₂₁ClN₆O₂S | 9.4 | 25 | 10.0 | |
| | N-(4-chlorobenzyl)-5-(4- | | | | | |
| | phenylpiperazine-1-carboxamido)- | | | | | |
| | 1,2,3-thiadiazole-4-carboxamide | | | | | |
| 86 | 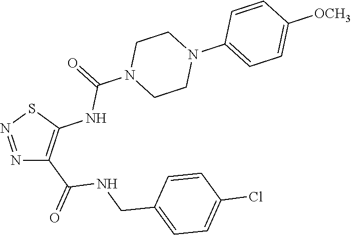 [SMILES: COc1ccc(N2CCN(C(=O)Nc3snnc3C(=O)NCc3ccc(Cl)cc3)CC2)cc1] | C₂₂H₂₃ClN₆O₃S | 32.6 | | | |
| | N-(4-chlorobenzyl)-5-(4-(4- | | | | | |
| | methoxyphenyl)piperazine-1- | | | | | |
| | carboxamido)-1,2,3-thiadiazole-4- | | | | | |
| | carboxamide | | | | | |
| 87 | 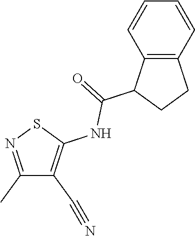 [SMILES: Cc1nsc(NC(=O)C2CCc3ccccc32)c1C#N] | C₁₅H₁₃N₃OS | 17 | 80 | | |
| | Indan-1-carboxylic acid (4-cyano- | | | | | |
| | 3-methyl-isothiazol-5-yl)-amide | | | | | |
| 89 | 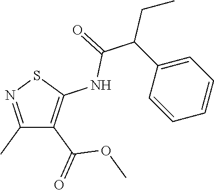 [SMILES: CCC(C(=O)Nc1snc(C)c1C(=O)OC)c1ccccc1] | C₁₆H₁₈N₂O₃S | 29.3 | | | |
| | 3-Methyl-5-(2-phenyl- | | | | | |
| | butyrylamino)-isothiazole-4- | | | | | |
| | carboxylic acid methyl ester | | | | | |
| 90 | 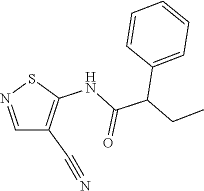 [SMILES: CCC(C(=O)Nc1sncc1C#N)c1ccccc1] | C₁₄H₁₃N₃OS | 21.8 | | | |
| | N-(4-Cyano-isothiazol-5-yl)-2- | | | | | |
| | phenyl-butyramide | | | | | |
| 95 | 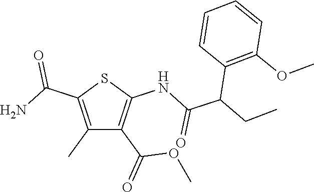 [SMILES: CCC(C(=O)Nc1sc(C(N)=O)c(C)c1C(=O)OC)c1ccccc1OC] | C₁₉H₂₂N₂O₅S | | | 21.2 | |
| | Methyl 2-(2-(2- | | | | | |
| | methoxyphenyl)butanamido)-5- | | | | | |
| | carbamoyl-4-methylthiophene-3- | | | | | |
| | carboxylate | | | | | |
| 96 | 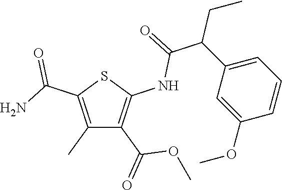 [SMILES: CCC(C(=O)Nc1sc(C(N)=O)c(C)c1C(=O)OC)c1cccc(OC)c1] | C₁₉H₂₂N₂O₅S | | | 56% @ 25 uM | |
| | Methyl 2-(2-(3- | | | | | |
| | methoxyphenyl)butanamido)-5- | | | | | |
| | carbamoyl-4-methylthiophene-3- | | | | | |
| | carboxylate | | | | | |
| 97 | 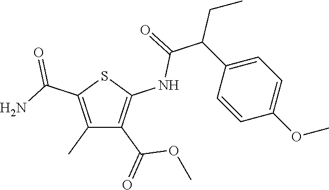 [SMILES: CCC(C(=O)Nc1sc(C(N)=O)c(C)c1C(=O)OC)c1ccc(OC)cc1] | C₁₉H₂₂N₂O₅S | | | 37% @ 25 uM | |
| | Methyl 2-(2-(4- | | | | | |
| | methoxyphenyl)butanamido)-5- | | | | | |
| | carbamoyl-4-methylthiophene-3- | | | | | |
| | carboxylate | | | | | |
| 98 | 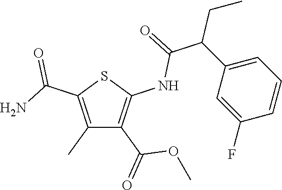 [SMILES: CCC(C(=O)Nc1sc(C(N)=O)c(C)c1C(=O)OC)c1cccc(F)c1] | C₁₈H₁₉FN₂O₄S | | | 18.0 | |
| | Methyl 2-(2-(3- | | | | | |
| | fluorophenyl)butanamido)-5- | | | | | |
| | carbamoyl-4-methylthiophene-3- | | | | | |
| | carboxylate | | | | | |
| 99 | 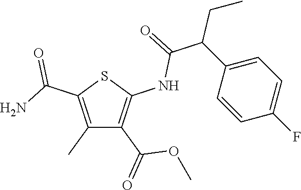 [SMILES: CCC(C(=O)Nc1sc(C(N)=O)c(C)c1C(=O)OC)c1ccc(F)cc1] | C₁₈H₁₉FN₂O₄S | 9.0 | 5.4 | 8.8 | 8.9 |
| | 5-Carbamoyl-2-[2-(4-fluoro- | | | | | |
| | phenyl)-butyrylamino]-4-methyl- | | | | | |
| | thiophene-3-carboxylic acid methyl | | | | | |
| | ester | | | | | |
| 106 | 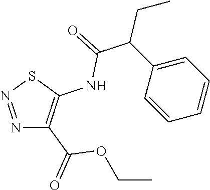 [SMILES: CCOC(=O)c1nnsc1NC(=O)C(CC)c1ccccc1] | C₁₅H₁₇N₃O₃S | | | 40% @ 25 uM | |
| | 5-(2-Phenyl-butyrylamino)- | | | | | |
| | [1,2,3]thiadiazole-4-carboxylic acid | | | | | |
| | ethyl ester | | | | | |
| 109 | 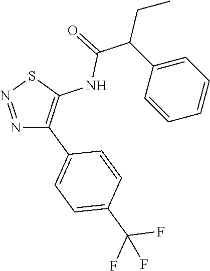 [SMILES: CCC(C(=O)Nc1snnc1-c1ccc(C(F)(F)F)cc1)c1ccccc1] | C₁₉H₁₆F₃N₃OS | | | 0.8 | 68.6 |
| | 2-Phenyl-N-[4-(4-trifluoromethyl- | | | | | |
| | phenyl)-[1,2,3]thiadiazol-5-yl]- | | | | | |
| | butyramide | | | | | |
| 110 | 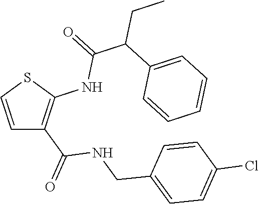 [SMILES: CCC(C(=O)Nc1sccc1C(=O)NCc1ccc(Cl)cc1)c1ccccc1] | C₂₂H₂₁ClN₂O₂S | | >200 | 8.0 | >100 |
| | 2-(2-Phenylbutanamido)-N-(4- | | | | | |
| | chlorobenzyl)thiophene-3- | | | | | |
| | carboxamide | | | | | |
| 111 | 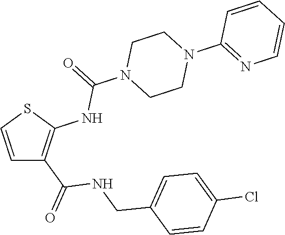 [SMILES: O=C(NCc1ccc(Cl)cc1)c1ccsc1NC(=O)N1CCN(c2ccccn2)CC1] | C₂₂H₂₂ClN₅O₂S | | | 7.0 | 63.3 |
| | 4-Pyridin-2-yl-piperazine-1- | | | | | |
| | carboxylic acid [3-(4-chloro- | | | | | |
| | benzylcarbamoyl)-thiophen-2-yl]- | | | | | |
| | amide | | | | | |
| 112 | 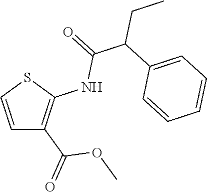 [SMILES: CCC(C(=O)Nc1sccc1C(=O)OC)c1ccccc1] | C₁₆H₁₇NO₃S | | | 59% @ 25 uM | |
| | Methyl 2-(2- | | | | | |
| | phenylbutanamido)thiophene-3- | | | | | |
| | carboxylate | | | | | |
| 114 | 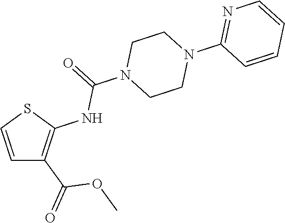 [SMILES: COC(=O)c1ccsc1NC(=O)N1CCN(c2ccccn2)CC1] | C₁₆H₁₈N₄O₃S | | | 32% @ 25 uM | |
| | Methyl 2-(4-(pyridin-2- | | | | | |
| | yl)piperazine-1- | | | | | |
| | carboxamido)thiophene-3- | | | | | |
| | carboxylate | | | | | |
| 115 | 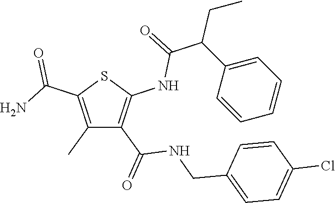 [SMILES: CCC(C(=O)Nc1sc(C(N)=O)c(C)c1C(=O)NCc1ccc(Cl)cc1)c1ccccc1] | C₂₄H₂₄ClN₃O₃S | | | 11.0 | |
| | 2-(2-Phenylbutanamido)-N3-(4- | | | | | |
| | chlorobenzyl)-4-methylthiophene- | | | | | |
| | 3,5-dicarboxamide | | | | | |
| 116 | 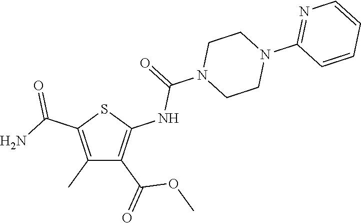 [SMILES: COC(=O)c1c(NC(=O)N2CCN(c3ccccn3)CC2)sc(C(N)=O)c1C] | C₁₈H₂₁N₅O₄S | | | 30% @ 25 uM | |
| | Methyl 2-(4-(pyridin-2- | | | | | |
| | yl)piperazine-1-carboxamido)-5- | | | | | |
| | carbamoyl-4-methylthiophene-3- | | | | | |
| | carboxylate | | | | | |
| 118 | 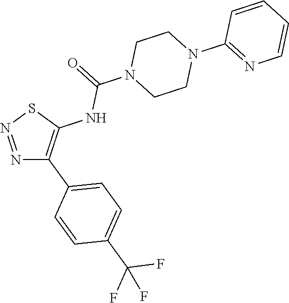 [SMILES: O=C(Nc1snnc1-c1ccc(C(F)(F)F)cc1)N1CCN(c2ccccn2)CC1] | C₁₉H₁₇F₃N₆OS | | 85.7 | 4.0 | |
| | 4-Pyridin-2-yl-piperazine-1- | | | | | |
| | carboxylic acid [4-(4- | | | | | |
| | trifluoromethyl-phenyl)- | | | | | |
| | [1,2,3]thiadiazol-5-yl]-amide | | | | | |
| 119 | 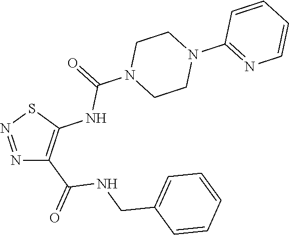 [SMILES: O=C(NCc1ccccc1)c1nnsc1NC(=O)N1CCN(c2ccccn2)CC1] | C₂₀H₂₁N₇O₂S | | | 10.3 | >100 |
| | 4-Pyridin-2-yl-piperazine-1- | | | | | |
| | carboxylic acid (4- | | | | | |
| | benzylcarbamoyl-[1,2,3]thiadiazol- | | | | | |
| | 5-yl)-amide | | | | | |
| 123 | 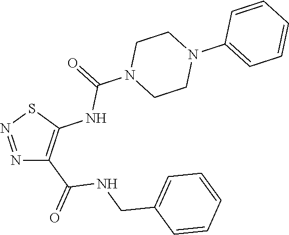 [SMILES: O=C(NCc1ccccc1)c1nnsc1NC(=O)N1CCN(c2ccccc2)CC1] | C₂₁H₂₂N₆O₂S | | | 44% @ 25 uM | |
| | 4-Phenyl-piperazine-1-carboxylic | | | | | |
| | acid (4-benzylcarbamoyl- | | | | | |
| | [1,2,3]thiadiazol-5-yl)-amide | | | | | |
| 124 | 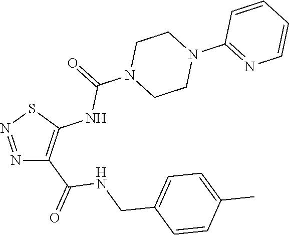 [SMILES: Cc1ccc(CNC(=O)c2nnsc2NC(=O)N2CCN(c3ccccn3)CC2)cc1] | C₂₁H₂₃N₇O₂S | | | 14.5 | |
| | 4-Pyridin-2-yl-piperazine-1- | | | | | |
| | carboxylic acid [4-(4-methyl- | | | | | |
| | benzylcarbamoyl)- | | | | | |
| | [1,2,3]thiadiazol-5-yl]-amide | | | | | |
| 127 | 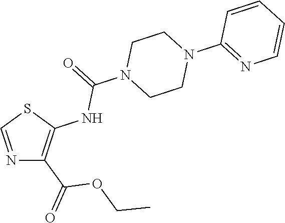 [SMILES: CCOC(=O)c1ncsc1NC(=O)N1CCN(c2ccccn2)CC1] | C₁₆H₁₉N₅O₃S | | | 29.5 | |
| | Ethyl 5-(4-(pyridin-2-yl)piperazine- | | | | | |
| | 1-carboxamido)thiazole-4- | | | | | |
| | carboxylate | | | | | |
| 130 | 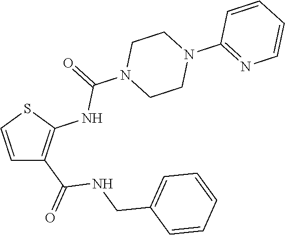 [SMILES: O=C(NCc1ccccc1)c1ccsc1NC(=O)N1CCN(c2ccccn2)CC1] | C₂₂H₂₃N₅O₂S | | | 32.8 | |
| | N-(3-(Benzylcarbamoyl)thiophen- | | | | | |
| | 2-yl)-4-(pyridin-2-yl)piperazine-1- | | | | | |
| | carboxamide | | | | | |
| 131 | 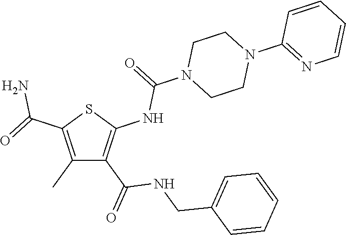 [SMILES: Cc1c(C(N)=O)sc(NC(=O)N2CCN(c3ccccn3)CC2)c1C(=O)NCc1ccccc1] | C₂₄H₂₆N₆O₃S | | | 31.8 | |
| | 2-(4-(pyridin-2-yl)piperazine-1- | | | | | |
| | carboxamido)-N3-benzyl-4- | | | | | |
| | methylthiophene-3,5- | | | | | |
| | dicarboxamide | | | | | |
| 132 | 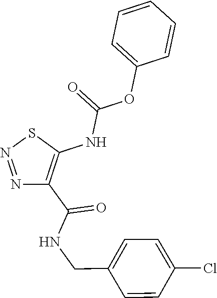 [SMILES: O=C(Nc1snnc1C(=O)NCc1ccc(Cl)cc1)Oc1ccccc1] | C₁₇H₁₃ClN₄O₃S | | | 16.4 | |
| | [4-(4-Chloro-benzylcarbamoyl)- | | | | | |
| | [1,2,3]thiadiazol-5-yl]-carbamic | | | | | |
| | acid phenyl ester | | | | | |
| 134 | 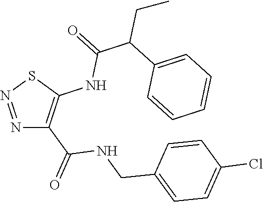 [SMILES: CCC(C(=O)Nc1snnc1C(=O)NCc1ccc(Cl)cc1)c1ccccc1] | C₂₀H₁₉ClN₄O₂S | | | 11.5 | |
| | 5-(2-Phenyl-butyrylamino)- | | | | | |
| | [1,2,3]thiadiazole-4-carboxylic acid | | | | | |
| | 4-chloro-benzylamide | | | | | |
| 135 | 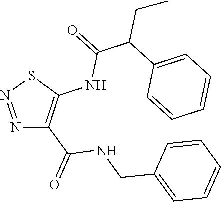 [SMILES: CCC(C(=O)Nc1snnc1C(=O)NCc1ccccc1)c1ccccc1] | C₂₀H₂₀N₄O₂S | | | 18.5 | |
| | 5-(2-Phenyl-butyrylamino)- | | | | | |
| | [1,2,3]thiadiazole-4-carboxylic acid | | | | | |
| | benzylamide | | | | | |
| 144 | 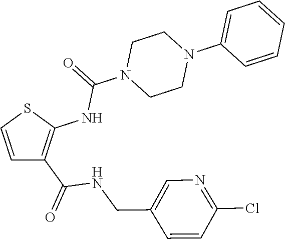 [SMILES: O=C(NCc1ccc(Cl)nc1)c1ccsc1NC(=O)N1CCN(c2ccccc2)CC1] | C₂₂H₂₂ClN₅O₂S | | | 38.0 | |
| | N-(3-((6-Chloropyridin-2- | | | | | |
| | yl)methylcarbamoyl)thiophen-2- | | | | | |
| | yl)-4-phenylpiperazine-1- | | | | | |
| | carboxamide | | | | | |
| 153 | 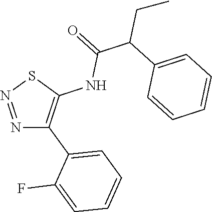 [SMILES: CCC(C(=O)Nc1snnc1-c1ccccc1F)c1ccccc1] | C₁₈H₁₆FN₃OS | | >200 | 10.2 | |
| | N-[4-(2-Fluoro-phenyl)- | | | | | |
| | [1,2,3]thiadiazol-5-yl]-2-phenyl- | | | | | |
| | butyramide | | | | | |
| 154 | 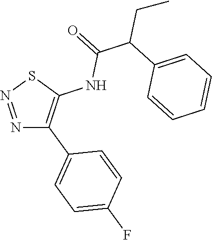 [SMILES: CCC(C(=O)Nc1snnc1-c1ccc(F)cc1)c1ccccc1] | C₁₈H₁₆FN₃OS | | >200 | 32.0 | |
| | N-[4-(4-Fluoro-phenyl)- | | | | | |
| | [1,2,3]thiadiazol-5-yl]-2-phenyl- | | | | | |
| | butyramide | | | | | |
| 155 | 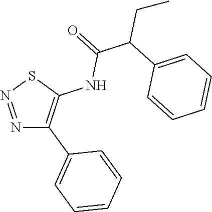 [SMILES: CCC(C(=O)Nc1snnc1-c1ccccc1)c1ccccc1] | C₁₈ H₁₇N3OS | | >100 | 16.2 | |
| | 2-Phenyl-N-(4-phenyl- | | | | | |
| | [1,2,3]thidiazol-5-yl)-butyramide | | | | | |
| 159 | 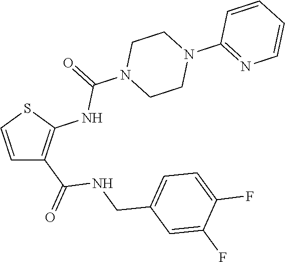 [SMILES: O=C(NCc1ccc(F)c(F)c1)c1ccsc1NC(=O)N1CCN(c2ccccn2)CC1] | C₂₄H₂₂F₅N₅O₄S | | | 12.8 | 118.0 |
| | N-(3-(3,4- | | | | | |
| | Difluorobenzylcarbamoyl)thiophen- | | | | | |
| | 2-yl)-4-(pyridin-2-yl)piperazine-1- | | | | | |
| | carboxamide | | | | | |
| 160 | 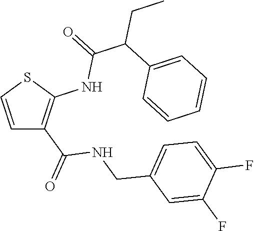 [SMILES: CCC(C(=O)Nc1sccc1C(=O)NCc1ccc(F)c(F)c1)c1ccccc1] | C₂₂H₂₀F₂N₂O₂S | | | 10.6 | 128.0 |
| | 2-(2-Phenylbutanamido)-N-(3,4- | | | | | |
| | difluorobenzyl)thiophene-3- | | | | | |
| | carboxamide | | | | | |
| 161 | 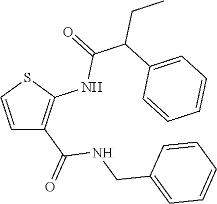 [SMILES: CCC(C(=O)Nc1sccc1C(=O)NCc1ccccc1)c1ccccc1] | C₂₂H₂₂N₂O₂S | | | 12.0 | 140.0 |
| | 2-(2-Phenylbutanamido)-N- | | | | | |
| | benzylthiophene-3-carboxamide | | | | | |
| 162 | 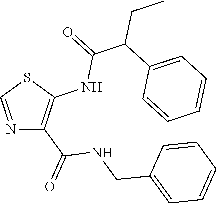 [SMILES: CCC(C(=O)Nc1scnc1C(=O)NCc1ccccc1)c1ccccc1] | C₂₁H₂₁N₃O₂S | | | 18.1 | |
| | 5-(2-Phenylbutanamido)-N- | | | | | |
| | benzylthiazole-4-carboxamide | | | | | |
| 163 | 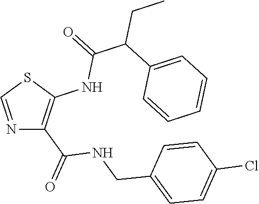 [SMILES: CCC(C(=O)Nc1scnc1C(=O)NCc1ccc(Cl)cc1)c1ccccc1] | C₂₁H₂₀ClN₃O₂S | | | 12.1 | >100 |
| | 5-(2-Phenylbutanamido)-N-(4- | | | | | |
| | chlorobenzyl)thiazole-4- | | | | | |
| | carboxamide | | | | | |
| 165 | 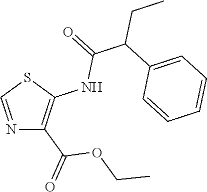 [SMILES: CCOC(=O)c1ncsc1NC(=O)C(CC)c1ccccc1] | C₁₆H₁₈N₂O₃S | | | 30.6% @ 25 uM | |
| | Ethyl 5-(2- | | | | | |
| | phenylbutanamido)thiazole-4- | | | | | |
| | carboxylate | | | | | |
| 167 | 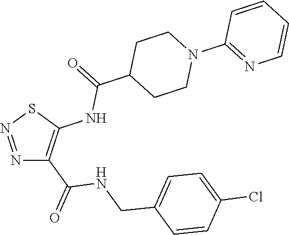 [SMILES: O=C(NCc1ccc(Cl)cc1)c1nnsc1NC(=O)C1CCN(c2ccccn2)CC1] | C₂₃H₂₂ClF₃N₆O₄S | | | 17.8 | 98.6 |
| | N-(4-(4-Chlorobenzylcarbamoyl)- | | | | | |
| | 1,2,3-thiadiazol-5-yl)-1-(pyridin-2- | | | | | |
| | yl)piperidine-4-carboxamide | | | | | |
| 168 | 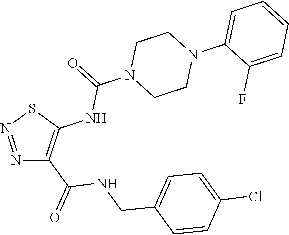 [SMILES: O=C(NCc1ccc(Cl)cc1)c1nnsc1NC(=O)N1CCN(c2ccccc2F)CC1] | C₂₁H₂₀ClFN₆O₂S | | | 11.9 | 26.3 |
| | 4-(2-Fluoro-phenyl)-piperazine-1- | | | | | |
| | carboxylic acid [4-(4-chloro- | | | | | |
| | benzylcarbamoyl)- | | | | | |
| | [1,2,3]thiadiazol-5-yl]-amide | | | | | |
| 169 | 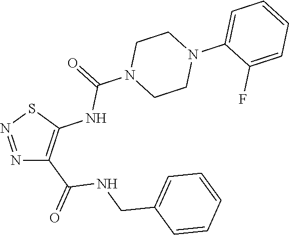 [SMILES: O=C(NCc1ccccc1)c1nnsc1NC(=O)N1CCN(c2ccccc2F)CC1] | C₂₁H₂₁FN₆O₂S | | | 32.7 | |
| | 4-(2-Fluoro-phenyl)-piperazine-1- | | | | | |
| | carboxylic acid (4- | | | | | |
| | benzylcarbamoyl-[1,2,3]thiadiazol- | | | | | |
| | 5-yl)-amide | | | | | |
| 170 | 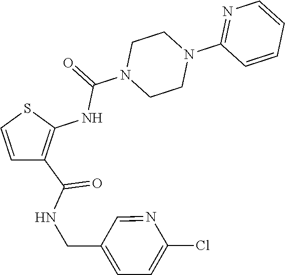 [SMILES: O=C(NCc1ccc(Cl)nc1)c1ccsc1NC(=O)N1CCN(c2ccccn2)CC1] | C₂₃H₂₂ClF₃N₆O₄S | | | 14.9 | >200 |
| | N-(3-((6-Chloropyridin-3- | | | | | |
| | yl)methylcarbamoyl)thiophen-2- | | | | | |
| | yl)-4-(pyridin-2-yl)piperazine-1- | | | | | |
| | carboxamide | | | | | |
| 172 | 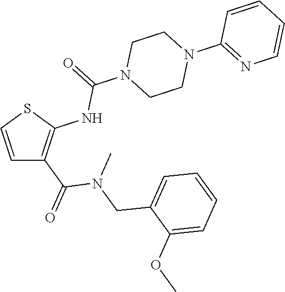 [SMILES: COc1ccccc1CN(C)C(=O)c1ccsc1NC(=O)N1CCN(c2ccccn2)CC1] | C₂₆H₂₈F₃N₅O₅S | | | 22.6 | 117.0 |
| | N-(3-(N-(2-methoxybenzyl)-N- | | | | | |
| | methylcarbamoyl)thiophen-2-yl)-4- | | | | | |
| | (pyridin-2-yl)piperazine-1- | | | | | |
| | carboxamide | | | | | |
| 173 | 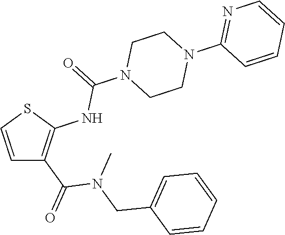 [SMILES: CN(Cc1ccccc1)C(=O)c1ccsc1NC(=O)N1CCN(c2ccccn2)CC1] | C₂₅H₂₆F₃N₅O₄S | | | 11.9 | >200 |
| | N-(3-(N-Benzyl-N- | | | | | |
| | methylcarbamoyl)thiophen-2-yl)-4- | | | | | |
| | (pyridin-2-yl)piperazine-1- | | | | | |
| | carboxamide | | | | | |
| 174 | 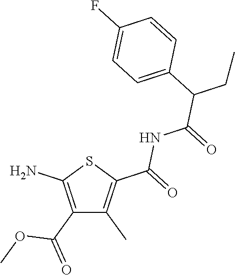 [SMILES: CCC(C(=O)NC(=O)c1sc(N)c(C(=O)OC)c1C)c1ccc(F)cc1] | C₁₈H₁₉FN₂O₄S | | | 20.5 | |
| | Methyl 5-(2-(4- | | | | | |
| | fluorophenyl)butanoylcarbamoyl)- | | | | | |
| | 2-amino-4-methylthiophene-3- | | | | | |
| | carboxylate | | | | | |
| 175 | 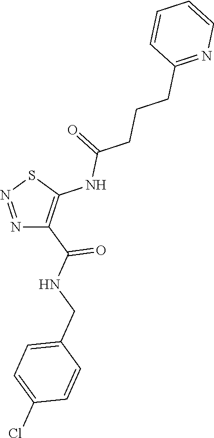 [SMILES: O=C(CCCc1ccccn1)Nc1snnc1C(=O)NCc1ccc(Cl)cc1] | C₂₁H₁₉ClF₃N₅O₄S | | | 15.5 | |
| | 5-(4-(Pyridin2-yl)butanamido)-N- | | | | | |
| | (4-chlorobenzyl)-1,2,3-thiadiazole- | | | | | |
| | 4-carboxamide | | | | | |
| 179 | 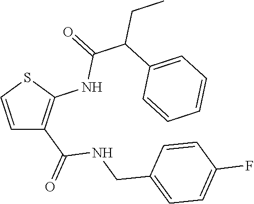 [SMILES: CCC(C(=O)Nc1sccc1C(=O)NCc1ccc(F)cc1)c1ccccc1] | C₂₂H₂₁FN₂O₂S | | | 9.5 | 51.3 |
| | 2-(2-Phenylbutanamido)-N-(4- | | | | | |
| | fluorobenzyl)thiophene-3- | | | | | |
| | carboxamide | | | | | |
| 180 | 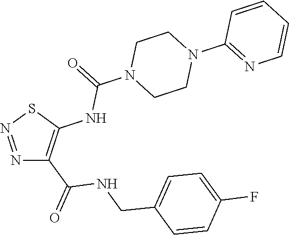 [SMILES: O=C(NCc1ccc(F)cc1)c1nnsc1NC(=O)N1CCN(c2ccccn2)CC1] | C₂₂H₂₁F₄N₇O₄S | | | 12.9 | 39.0 |
| | N-(4-(4-Fluorobenzylcarbamoyl)- | | | | | |
| | 1,2,3-thiadiazol-5-yl)-4-(pyriidn-2- | | | | | |
| | yl)piperazine-1-carboxamide | | | | | |
| 181 | 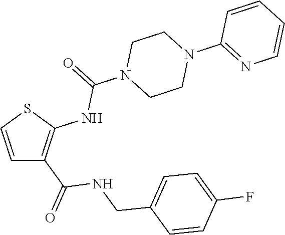 [SMILES: O=C(NCc1ccc(F)cc1)c1ccsc1NC(=O)N1CCN(c2ccccn2)CC1] | C₂₄H₂₃F₄N₅O₄S | | | 16.0 | 135.0 |
| | N-(3-(4- | | | | | |
| | Fluorobenzylcarbamoyl)thiophen- | | | | | |
| | 2-yl)-4-(pyridin-2-yl)piperazine-1- | | | | | |
| | carboxamide | | | | | |
| 182 | 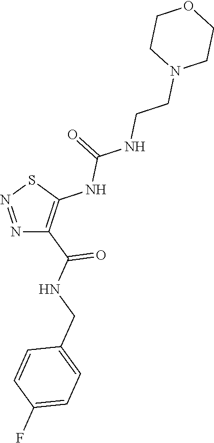 [SMILES: O=C(NCCN1CCOCC1)Nc1snnc1C(=O)NCc1ccc(F)cc1] | C₁₇H₂₁FN₆O₃S | | | 12.4 | >100 |
| | 1-(4-(4-Fluorobenzylcarbamoyl)- | | | | | |
| | 1,2,3-thiadiazol-5-yl)-3-(2- | | | | | |
| | morpholinoethyl)urea | | | | | |
| 183 | 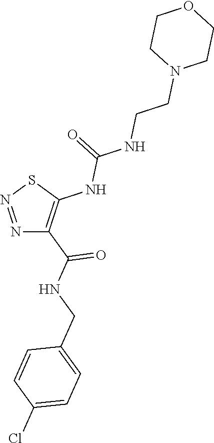 [SMILES: O=C(NCCN1CCOCC1)Nc1snnc1C(=O)NCc1ccc(Cl)cc1] | C₁₇H₂₁ClN₆O₃S | | | 11.9 | >100 |
| | 1-(4-(4-Chlorobenzylcarbamoyl)- | | | | | |
| | 1,2,3-thiadiazol-5-yl)-3-(2- | | | | | |
| | morpholinoethyl)urea | | | | | |
| 184 | 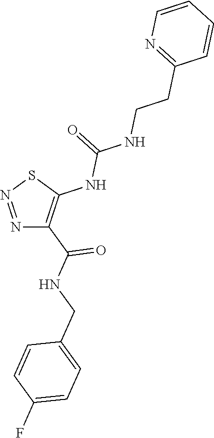 [SMILES: O=C(NCCc1ccccn1)Nc1snnc1C(=O)NCc1ccc(F)cc1] | C₁₈H₁₇FN₆O₂S | | | 22.8 | 60.0 |
| | 1-(4-(4-Fluorobenzylcarbamoyl)- | | | | | |
| | 1,2,3-thiadiazol-5-yl)-3-(2-(pyridin- | | | | | |
| | 2-yl)ethyl)urea | | | | | |
| 185 | 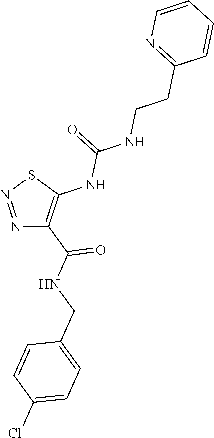 [SMILES: O=C(NCCc1ccccn1)Nc1snnc1C(=O)NCc1ccc(Cl)cc1] | C₁₈H₁₇ClN₆O₂S | | | 9.6 | 64.7 |
| | 1-(4-(4-Chlorobenzylcarbamoyl)- | | | | | |
| | 1,2,3-thiadiazol-5-yl)-3-(2-(pyridin- | | | | | |
| | 2-yl)ethyl)urea | | | | | |
| 186 | 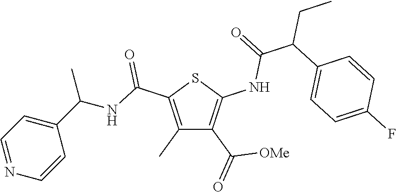 [SMILES: CCC(C(=O)Nc1sc(C(=O)NC(C)c2ccncc2)c(C)c1C(=O)OC)c1ccc(F)cc1] | C₂₅H₂₆FN₃O₄S | | 39.0 | 20.0 | |
| | methyl 2-(2-(4- | | | | | |
| | fluorophenyl)butanamido)-4- | | | | | |
| | methyl-5-((1-(pyridin-4- | | | | | |
| | yl)ethyl)carbamoyl)thiophene-3- | | | | | |
| | carboxylate | | | | | |
| 187 | 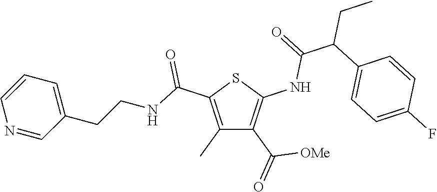 [SMILES: CCC(C(=O)Nc1sc(C(=O)NCCc2cccnc2)c(C)c1C(=O)OC)c1ccc(F)cc1] | C₂₅H₂₆FN₃O₄S | | 11.6 | 13.8 | |
| | methyl 2-(2-(4- | | | | | |
| | fluorophenyl)butanamido)-4- | | | | | |
| | methyl-5-((2-(pyridin-3- | | | | | |
| | yl)ethyl)carbamoyl)thiophene-3- | | | | | |
| | carboxylate | | | | | |
| 188 | 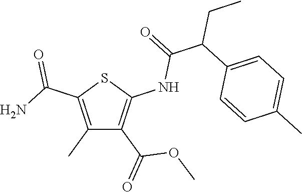 [SMILES: CCC(C(=O)Nc1sc(C(N)=O)c(C)c1C(=O)OC)c1ccc(C)cc1] | C₁₉H₂₂N₂O₄S | | | 64.1 | |
| | methyl 5-carbamoyl-4-methyl-2-(2- | | | | | |
| | (p-tolyl)butanamido)thiophene-3- | | | | | |
| | carboxylate | | | | | |
| 189 | 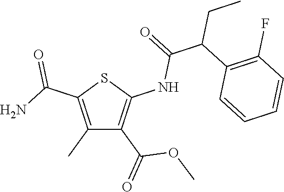 [SMILES: CCC(C(=O)Nc1sc(C(N)=O)c(C)c1C(=O)OC)c1ccccc1F] | C₁₈H₁₉FN₂O₄S | | | 15.2 | |
| | methyl 5-carbamoyl-2-(2-(2- | | | | | |
| | fluorophenyl)butanamido)-4- | | | | | |
| | methylthiophene-3-carboxylate | | | | | |
| 190 | 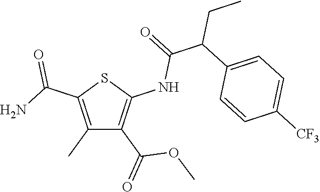 [SMILES: CCC(C(=O)Nc1sc(C(N)=O)c(C)c1C(=O)OC)c1ccc(C(F)(F)F)cc1] | C₁₉H₁₉F₃N₂O₄S | | | 4.8 | |
| | methyl 5-carbamoyl-4-methyl-2-(2- | | | | | |
| | (4- | | | | | |
| | (trifluoromethyl)phenyl)butanamido) | | | | | |
| | thiophene-3-carboxylate | | | | | |
| 191 | 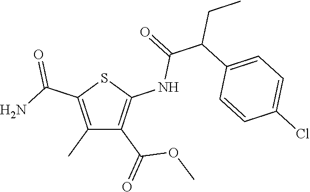 [SMILES: CCC(C(=O)Nc1sc(C(N)=O)c(C)c1C(=O)OC)c1ccc(Cl)cc1] | C₁₈H₁₉ClN₂O₄S | | | 11.4 | |
| | methyl 5-carbamoyl-2-(2-(4- | | | | | |
| | chlorophenyl)butanamido)-4- | | | | | |
| | methylthiophene-3-carboxylate | | | | | |
| 192 | 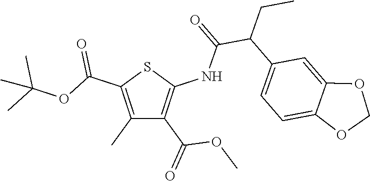 [SMILES: CCC(C(=O)Nc1sc(C(=O)OC(C)(C)C)c(C)c1C(=O)OC)c1ccc2c(c1)OCO2] | C₂₃H₂₇NO₇S | | | >50 | |
| | 2-(tert-butyl) 4-methyl-5-(2- | | | | | |
| | (benzo[d][1,3]dioxol-5- | | | | | |
| | yl)butanamido)-3-methylthiophene- | | | | | |
| | 2,4-dicarboxylate | | | | | |
| 193 | 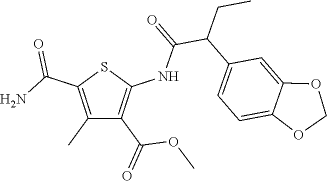 [SMILES: CCC(C(=O)Nc1sc(C(N)=O)c(C)c1C(=O)OC)c1ccc2c(c1)OCO2] | C₁₉H₂₀N₂O₆S | | | >50 | |
| | methyl 2-(2-(benzo[d][1,3]dioxol- | | | | | |
| | 5-yl)butanamido)-5-carbamoyl-4- | | | | | |
| | methylthiophene-3-carboxylate | | | | | |
| 194 | 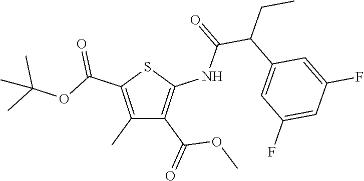 [SMILES: CCC(C(=O)Nc1sc(C(=O)OC(C)(C)C)c(C)c1C(=O)OC)c1cc(F)cc(F)c1] | C₂₂H₂₅F₂NO₅S | | | >50 | |
| | 2-(tert-butyl) 4-methyl 5-(2-(3,5- | | | | | |
| | difluorophenyl)butanamido)-3- | | | | | |
| | methylthiophene-2,4-dicarboxylate | | | | | |
| 195 | 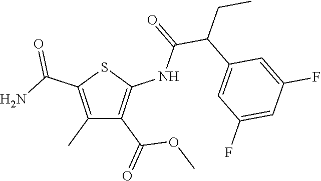 [SMILES: CCC(C(=O)Nc1sc(C(N)=O)c(C)c1C(=O)OC)c1cc(F)cc(F)c1] | C₁₈H₁₈F₂N₂O₄S | | | 25.8 | |
| | methyl 5-carbamoyl-2-(2-(3,5- | | | | | |
| | difluorophenyl)butanamido)-4- | | | | | |
| | methylthiophene-3-carboxylate | | | | | |
| 196 | 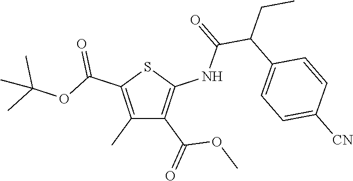 [SMILES: CCC(C(=O)Nc1sc(C(=O)OC(C)(C)C)c(C)c1C(=O)OC)c1ccc(C#N)cc1] | C₂₃H₂₆N₂O₅S | | | | |
| | 2-(tert-butyl) 4-methyl 5-(2-(4- | | | | | |
| | cyanophenyl)butanamido)-3- | | | | | |
| | methylthiophene-2,4-dicarboxylate | | | | | |
| 197 | 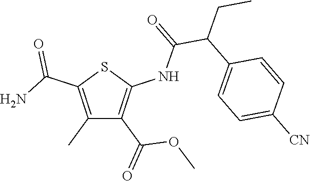 [SMILES: CCC(C(=O)Nc1sc(C(N)=O)c(C)c1C(=O)OC)c1ccc(C#N)cc1] | C₁₉H₁₉N₃O₄S | | | 42.0 | |
| | methyl 5-carbamoyl-2-(2-(4- | | | | | |
| | cyanophenyl)butanamido)-4- | | | | | |
| | methylthiophene-3-carboxylate | | | | | |
| 198 | 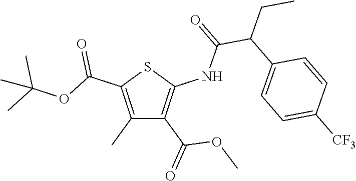 [SMILES: CCC(C(=O)Nc1sc(C(=O)OC(C)(C)C)c(C)c1C(=O)OC)c1ccc(C(F)(F)F)cc1] | C₂₃H₂₆F₃NO₅S | | | >50 | |
| | 2-(tert-butyl) 4-methyl 3-methyl-5- | | | | | |
| | (2-(4- | | | | | |
| | (trifluoromethyl)phenyl)butanamido) | | | | | |
| | thiophene-2,4-dicarboxylate | | | | | |
| 199 | 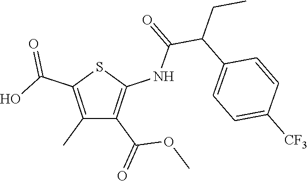 [SMILES: CCC(C(=O)Nc1sc(C(=O)O)c(C)c1C(=O)OC)c1ccc(C(F)(F)F)cc1] | C₁₉H₁₈F₃NO₅S | | | 17.9 | |
| | 4-(methoxycarbonyl)-3-methyl-5- | | | | | |
| | (2-(4- | | | | | |
| | (trifluoromethyl)phenyl)butanamido) | | | | | |
| | thiophene-2-carboxylic acid | | | | | |
| 200 | 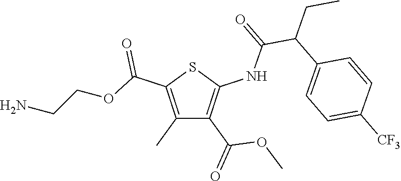 [SMILES: CCC(C(=O)Nc1sc(C(=O)OCCN)c(C)c1C(=O)OC)c1ccc(C(F)(F)F)cc1] | C₂₁H₂₃F₃N₂O₅S | | | 5.1 | |
| | 2-(2-aminoethyl) 4-methyl 3- | | | | | |
| | methyl-5-(2-(4- | | | | | |
| | (trifluoromethyl)phenyl)butanamido) | | | | | |
| | thiophene-2,4-dicarboxylate | | | | | |
| 201 | 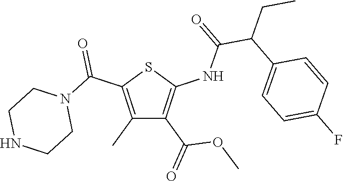 [SMILES: CCC(C(=O)Nc1sc(C(=O)N2CCNCC2)c(C)c1C(=O)OC)c1ccc(F)cc1] | C₂₂H₂₆FN₃O₄S | | | >25 | |
| 202 | 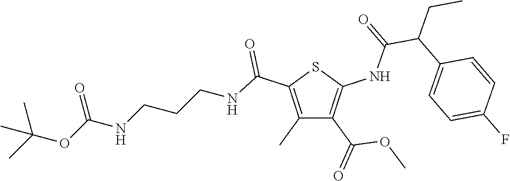 [SMILES: CCC(C(=O)Nc1sc(C(=O)NCCCNC(=O)OC(C)(C)C)c(C)c1C(=O)OC)c1ccc(F)cc1] | C₂₆H₃₄FN₃O₆S | | | >50 | |
| | methyl 5-((3-((tert- | | | | | |
| | butoxycarbonyl)amino)propyl)carba- | | | | | |
| | moyl)-2-(2-(4- | | | | | |
| | fluorophenyl)butanamido)-4- | | | | | |
| | methylthiophene-3-carboxylate | | | | | |
| 203 | 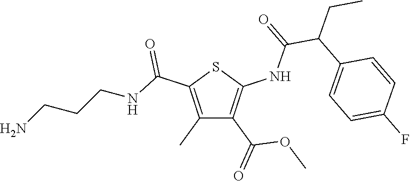 [SMILES: CCC(C(=O)Nc1sc(C(=O)NCCCN)c(C)c1C(=O)OC)c1ccc(F)cc1] | C₂₁H₂₆FN₃O₄S | | | 20 | |

Compound 199 (CC₅₀ = >100 μM; ATP assay)

### Example 6: In Vitro Skin Penetration Study

A non-GLP in vitro skin penetration study was conducted using compounds 99 and 111. The project included pre-formulation studies to produce five prototype formulations for each compound, development of a robust method for measuring percutaneous penetration and a skin permeation study using human cadaver skin in a vertical diffusion (Franz) cell model. The diffusion chambers were designed to maintain the skin sections at a temperature and humidity that represents typical in vivo conditions. The vertical diffusion cell human skin dose model has historic precedent for accurately predicting in vivo percutaneous absorption kinetics.

TABLE 3
Formulation compounds

| Cmpd | Structure | Salt | Purity^{a} | cLogD | TPSA | pKa^{b} |
|---|---|---|---|---|---|---|
| 111 | 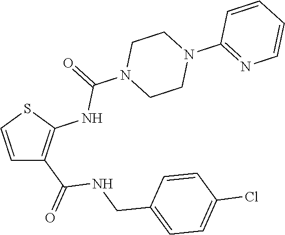 [SMILES: O=C(NCc1ccc(Cl)cc1)c1ccsc1NC(=O)N1CCN(c2ccccn2)CC1] | HCl | >99% | 4.40 | 77.57 | 6.42 |
| 99 | 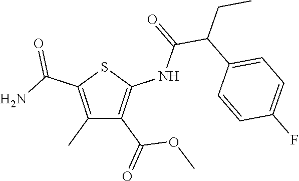 [SMILES: CCC(C(=O)Nc1sc(C(N)=O)c(C)c1C(=O)OC)c1ccc(F)cc1] | Free base | >99% | 4.27 | 98.49 | −1.77 |
| 99b | 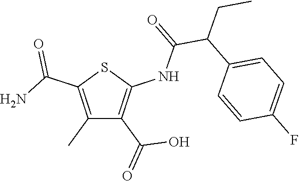 [SMILES: CCC(C(=O)Nc1sc(C(N)=O)c(C)c1C(=O)O)c1ccc(F)cc1] | Free base | >99% | 0.56 | 109.49 | −1.77 |

^{a}determined by NMR and LC/MS;
^{b}pKa calculated for HCl salt of compound shown.

Compound solubilities for compounds 99 and 111 were evaluated in 42 excipients by visual appearance and HPLC. Based on these results, 10 theoretical solvent mixtures were P designed for each compound and of these 10 mixtures, 3 mixtures were prepared and tested for solubility with a target final concentration of 10 mg/mL (10). The samples were prepared by dissolving 1.400 of the API in the mixture, stirring overnight, filtering and then analyzing the solution by HPLC for recovery and target concentration.

TABLE 4A
Formulations of compound 111

| Excipient | F1 | F2 | F3 | F4 | F5 |
|---|---|---|---|---|---|
| Compound 111 | 1.00 | 1.00 | 1.00 | 1.00 | 1.00 |
| Water (qs) | — | — | — | 13.30 | — |
| PEG 400 (qs) | 24.30 | 26.30 | 34.00 | — | — |
| PEG 300 (qs) | 40.00 | 40.00 | 40.00 | 57.00 | 48.00 |
| Propylene glycol | 10.00 | 10.00 | 10.00 | 10.00 | 10.00 |
| Benzyl alcohol | 2.70 | 2.70 | 2.00 | 2.70 | 1.50 |
| Super refined- | 5.00 | 5.00 | 5.00 | 5.00 | — |
| polysorbate 80 | | | | | |
| Transcutol ® P | 5.00 | 5.00 | 5.00 | 5.00 | 10.00 |
| (DEGEE) | | | | | |
| Isopropyl myristate | 2.00 | — | 2.00 | 2.00 | — |
| Ethanol (200 proof) | — | — | — | — | 8.50 |
| Diisopropyl adipate | — | — | — | — | 10.00 |
| C₁₂₁₅ alkyl lactate | — | — | — | — | 10.00 |
| Sepineo ™ P600 | — | — | — | 4.00 | — |
| Hydroxypropyl cellulose | — | — | 1.00 | — | 1.00 |
| (4500-6000 Cp) | | | | | |
| Polyethylene glycol 4000 | 10.00 | 10.00 | — | — | — |
| Total | 100.00 | 100.00 | 100.00 | 100.00 | 100.00 |

^{a}values provided as % w/w

TABLE 4B
Formulations of compound 99

| Excipient | F6 | F7 | F8 | F9 | F10 |
|---|---|---|---|---|---|
| Compound 99 | 1.00 | 1.00 | 1.00 | 1.00 | 1.00 |
| Water (qs) | — | — | — | — | 5.00 |
| PEG 400 (qs) | 28.30 | 30.30 | 19.30 | — | — |
| Propylene glycol | 20.00 | 20.00 | 20.00 | — | — |
| Benzyl alcohol | 2.70 | 2.70 | 2.70 | — | — |
| Super refined- | 5.00 | 5.00 | 5.00 | — | — |
| polysorbate 80 | | | | | |
| Transcutol ® P | 25.00 | 25.00 | 25.00 | 40.00 | 42.50 |
| (DEGEE) | | | | | |
| Isopropyl myristate | 2.00 | — | 2.00 | — | — |
| Ethanol (200 proof) | — | — | — | 28.00 | 25.00 |
| Diisopropyl adipate | — | — | — | 10.00 | 10.00 |
| C₁₂₋₁₅ alkyl lactate | — | — | — | 10.00 | 5.00 |
| Super refined dimethyl | 10.00 | 10.00 | 10.00 | 10.00 | 10.00 |
| isosorbide | | | | | |
| PEG 40 HCO | 5.00 | 5.00 | 5.00 | — | — |
| Hydroxypropyl cellulose | 1.00 | 1.00 | — | 1.00 | 1.5 |
| (4500-6000 Cp) | | | | | |
| Polyethylene glycol 4000 | — | — | 10.00 | — | — |
| Total | 100.00 | 100.00 | 100.00 | 100.00 | 100.00 |

^{a}values provided as % w/w

For compound 111, all 3 mixtures could achieve a 100 target concentration and 2 mixtures could reach the 1% target concentration for 99. From these results, 5 prototype formulations were designed for each compound and include gels and ointments with variations in levels of permeation enhancers (Tables 5A-5B). An optimized mixing protocol was developed and 1% target concentrations were reached in all 5 formulations for each compound. For compound 111, F1 and F2 are ointments and F3, F4 and F5 are gels. For compound 99, F8 is an ointment and F6, F7, F9 and F10 are gels. A pilot 2 week stability test was done at room temperature with 3 formulations for both compounds 99 and 111. Percent recoveries between the T=0 and T=2 week samples showed <2.0% API loss for all formulations indicating good product stability.

Five formulations for compounds 99 and 111 were tested in triplicate in an in vitro skin penetration (IVPT) study to measure levels of compound in the stratum corneum, epidermis, dermis and receptor medium using human dermatomed skin, wherein a Franz cell apparatus used, as described elsewhere herein. For this study, human torso skin was used rather than a membrane. Initially, methods development was done to identify the receiving medium in the reservoir of the Franz cell apparatus which provided the greatest compound solubility so that percentages of full penetration would not be underestimated due to poor solubility. A total of 6 receptor media buffers were evaluated and 1×PBS/4% BSA was found the give the highest solubility.

Furthermore, solubility values were sufficient to detect up to 143% and 90% of the applied amounts of 99 and 111, respectively, in a typical IVPT study which far exceeds the amount of compound expected to fully penetrate skin.

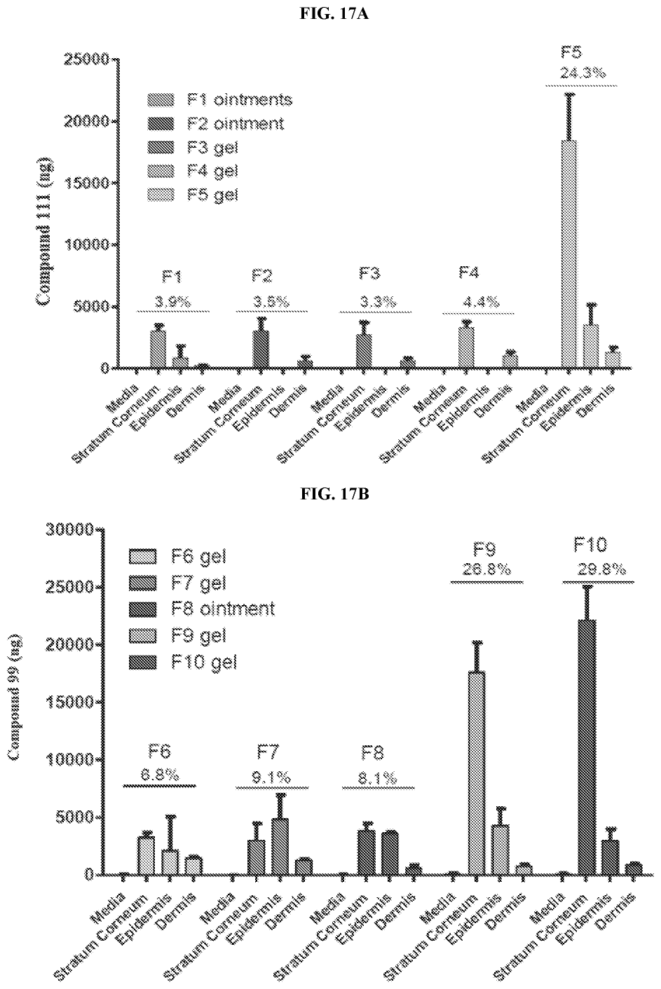

The total percentage of compound recovered in all four samples is shown for each formulation (FIGS. 17A-17B). For formulations F1-F4, <5% of 111 was recovered in the four samples while most presumably stayed in the formulation at the skin surface and was washed away. The F5 gel showed the greatest cutaneous penetration with the highest levels of compound 111 being found in the stratum corneum. Although lower than the stratum corneum, substantial levels of 111 were also recovered in the epidermis and dermis with F5 (5.1% of total applied).

While stratum corneum levels of 99 were highest with F9 and F10, the epidermal and dermal levels were more comparable across all five formulations with the highest levels being obtained with F7 (6.1% of total applied) and F9 (5.1% of total applied). Negligible amounts were found in the receptor medium with all formulations for each compound.

Only two formulations with 111 showed full skin penetration (recovery in the receptor medium) and the highest values were obtained with F5 (4.2 ng or 0.004% of total applied). All five formulations showed full skin penetration with 99 and the highest values were obtained with F9 (95 ng or 0.09% of total applied). Interestingly, F7 showed the highest penetration into the epidermis and dermis but showed the least full penetration with only 18.9 ng (0.018% of total applied) being recovered in the receptor medium. Only very low levels of the 99 acid analog (99b) were found in the epidermis with all 5 formulations (3.4 to 10 ng total) indicating a low degree of metabolic conversion in this study.

Molluscum contagiosum grows exclusively in the epidermis of human skin, the goal is to deliver effective anti-viral levels of compound to the epidermis while minimizing systemic exposure. To better evaluate the significance of the compound levels for 99 and 111 obtained with the more skin penetrant formulations, weight amounts (i.e., ng/mg of tissue sample) were converted to μM levels by assuming 1 g of tissue is equal to 1 mL. It is recognized that there are considerable variables in predicting anti-viral concentrations of compound in tissues including the presence of multiple tissue compartments (e.g., cells, cell types, interstitial fluids, matrix, differing protein and lipid concentrations) and their effects on compound distributions and activities but it is believed that this is a useful first step in assessing whether there is a realistic probability of achieving effective compound concentrations in the target tissue after topical delivery.

TABLE 5A
Compound skin levels (compound 111)

| | | Conc. | Avg. conc. | Avg. conc. |
|---|---|---|---|---|
| Formulation | Skin Layer | (ng/mg) | (ng/mg) | (μM) |
| F1 ointment | Epidermis | 105.6 | 152.4 | 334.2 |
| F1 ointment | Epidermis | 0.6 | | |
| F1 ointment | Epidermis | 351.0 | | |
| F1 ointment | Dermis | 1.3 | 1.7 | 3.8 |
| F1 ointment | Dermis | 2.1 | | |
| F1 ointment | Dermis | 1.7 | | |
| F5 gel | Epidermis | 488.8 | 511.3 | 1121.4 |
| F5 gel | Epidermis | 379.6 | | |
| F5 gel | Epidermis | 665.6 | | |
| F5 gel | Dermis | 10.6 | 13.6 | 29.8 |
| F5 gel | Dermis | 14.4 | | |
| F5 gel | Dermis | 15.8 | | |

TABLE 5B
Compound skin levels (compound 99)

| | | Conc. | Avg. conc. | Avg. conc. |
|---|---|---|---|---|
| Formulation | Skin Layer | (ng/mg) | (ng/mg) | (μM) |
| F6 gel | Epidermis | 0.0 | 513.5 | 1357.0 |
| F6 gel | Epidermis | — | | |
| F6 gel | Epidermis | 1027.0 | | |
| F6 gel | Dermis | 12.8 | 13.5 | 29.6 |
| F6 gel | Dermis | 10.8 | | |
| F6 gel | Dermis | 16.9 | | |
| F7 gel | Epidermis | 1040.0 | 835.5 | 2207.8 |
| F7 gel | Epidermis | 1008.8 | | |
| F7 gel | Epidermis | 457.6 | | |
| F7 gel | Dermis | 13.5 | 13.6 | 36.0 |
| F7 gel | Dermis | 15.5 | | |
| F7 gel | Dermis | 11.9 | | |
| F8 gel | Epidermis | 608.4 | 617.9 | 1632.9 |
| F8 gel | Epidermis | 657.8 | | |
| F8 gel | Epidermis | 587.6 | | |
| F8 gel | Dermis | 16.9 | 13.1 | 34.7 |
| F8 gel | Dermis | 9.4 | | |
| F8 gel | Dermis | — | | |
| F9 gel | Epidermis | 1125.8 | 903.9 | 2388.7 |
| F9 gel | Epidermis | 980.2 | | |
| F9 gel | Epidermis | 605.8 | | |
| F9 gel | Dermis | 13.6 | 18.2 | 48.0 |
| F9 gel | Dermis | 24.0 | | |
| F9 gel | Dermis | 16.9 | | |
| F10 gel | Epidermis | 410.8 | 515.7 | 1362.7 |
| F10 gel | Epidermis | 670.8 | | |
| F10 gel | Epidermis | 465.4 | | |
| F10 gel | Dermis | 11.9 | 13.0 | 34.4 |
| F10 gel | Dermis | 15.4 | | |
| F10 gel | Dermis | 11.8 | | |

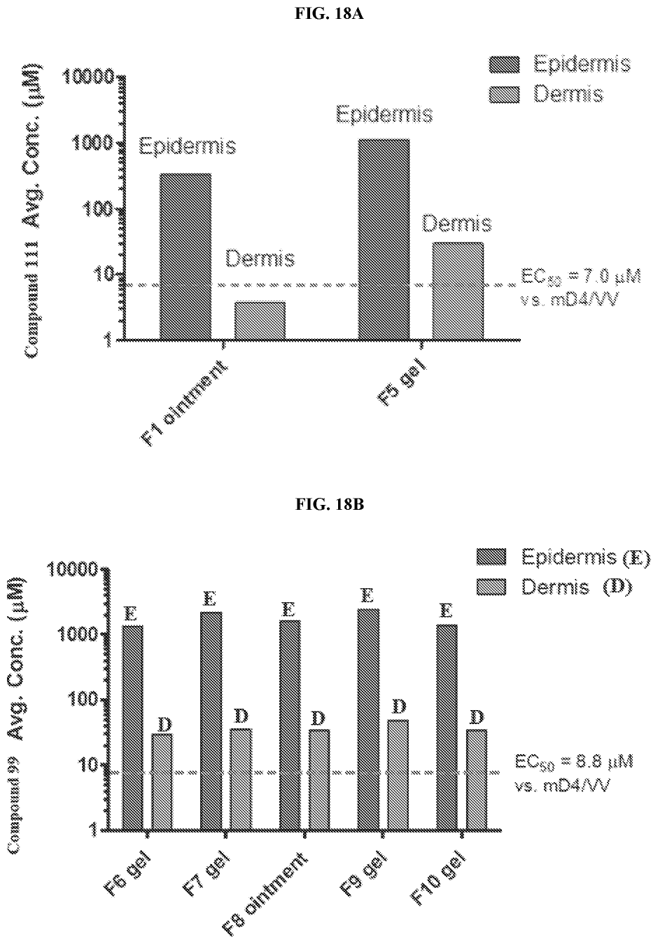

The average ng/mL and calculated average μM concentrations of compound in the epidermis and dermis obtained with formulations F1 and F5 with 111 and F6-F10 with 99 are provided herein (Tables 6A-6B). These data are also represented graphically in FIGS. 18A-18B, wherein the average μM concentrations of compound are plotted for each formulation.

Recognizing that the anti-viral activities of compounds 111 and 99 in cellular plaque assays against the hybrid mD4/vaccinia hybrid virus have EC₅₀ values of 7.0 μM and 8.8 μM, respectively, the epidermal concentrations of 111 in F5 and 99 in all 5 formulations exceed the EC₅₀ concentrations by at least 2 orders of magnitude.

Thus, formulations for compounds 111 and 99 have been confirmed by in vitro skin penetration studies that appear to deliver effective anti-viral compound concentrations to the epidermis of human cadaver skin after topical administration. Full penetration through all skin layers is low, consistent with low systemic exposure and metabolic conversion of 99 to its acid analog is negligible.

A stability study was performed at RT and 40° C. with 99 formulated in the F7 gel (8 mg/mL). A 98% recovery of 99 was obtained over the 3-month test period, indicating excellent stability.

Additionally, an acute dermal irritancy study was done in rabbits with 10 mg/mL of 99 contained within the F7 gel formulation. Briefly, gel was applied to shaved skin areas, covered for 24 hours and irritancy was measured daily for 3 days after uncovering using the Draize scoring system. The F7 gel formulation containing 99 was scored as a non-irritant. Similarly, gel formulated 99 was scored as a non-irritant on human cadaver skin.

Sequence Listings

SEQ ID NO: 1 mD4
MLRERALRAAPHVLRYHEDWEPVAEPLADAYAEVAPWLLRDRTEPAPERFFRQLELPLRDKR
VCIVGIDPYPEGATGVPFESPDFSKKTARALAAAAARAAEHGGCRRVSAYRNYDFRGVQGVL
AWNYYLSCRRGETKSHAMHWERIARMLLAHIARFVRVFYFLGRSDFGGVRAKLTAPVTLLVG
YHPAARGGQFESERTLEILNVLLELHGLAPVDWQGFVPL
SEQ ID NO: 2 VD4
MNSVTVSHAPYTITYHDDWEPVMSQLVEFYNEVASWLLRDETSPIPDKFFIQLKQPLRNKRV
CVCGIDPYPKDGTGVPFESPNFTKKSIKEIASSISRLTGVIDYKGYNLNIIDGVIPWNYYLS
CKLGETKSHAIYWDKISKLLLQHITKHVSVLYCLGKTDFSNIRAKLESPVTTIVGYHPAARD
RQFEKDRSFEIINVLLELDNKAPINWAQGFIY

### ENUMERATED EMBODIMENTS

The following exemplary embodiments are provided, the numbering of which is not to be construed as designating levels of importance:

Embodiment 1 provides a compound of formula (I), or a salt, solvate, enantiomer, diastereoisomer, geometric isomer, or tautomer thereof:

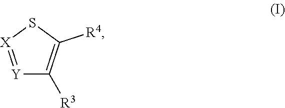

wherein:
X is CR¹ or N;
Y is CR² or N;
R¹ is H, optionally substituted C₁-C₆ alkyl, —C(═O)NR⁶R⁶, —C(═O)OR⁶, or R⁸;
R² is H or optionally substituted C₁-C₆ alkyl;
R³ is H, —CN, —C(═O)OR⁶, —C(═O)NR⁶R⁶, optionally substituted phenyl, or optionally substituted C₁-C₆ alkyl;
R⁴ is —C(═O)OR⁶ or R⁸;
each occurrence of R⁵ is independently optionally substituted C₁-C₆ alkyl or optionally substituted phenyl;
each occurrence of R⁶ is independently H or optionally substituted C₁-C₆ alkyl,
   or two R⁶ combine with the N atom to which both are bound to form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷;
each occurrence of R⁷ is independently optionally substituted C₁-C₆ alkyl, optionally substituted cycloalkyl, CH (optionally substituted heterocyclyl)(R⁵), CH(R⁵)(R⁵), or optionally substituted 4-7 membered heterocyclyl,
   or R⁶ and R⁷ combine with the N atom to which both are bound to form
   optionally substituted 4-7 membered heterocyclyl;
      with the proviso that (I) comprises a single R⁸; and
      with the proviso that the compound is not selected from the group consisting of Compounds 1, 2, 4, 5, 8, 9, 15, 16, 17, 19, 24, 26, 29, 31, 40, 66, 72, 76, 77, 78, 83, 84, and 86.

Embodiment 2 provides the compound of Embodiment 1, wherein X is CR¹ and Y is CR².

Embodiment 3 provides the compound of Embodiment 1, wherein X is N and Y is N.

Embodiment 4 provides the compound of Embodiment 1, wherein X is CR¹ and Y is N.

Embodiment 5 provides the compound of Embodiment 1, wherein X is N and Y is CR².

Embodiment 6 provides the compound of any one of Embodiments 1-5, which is selected from the group consisting of:

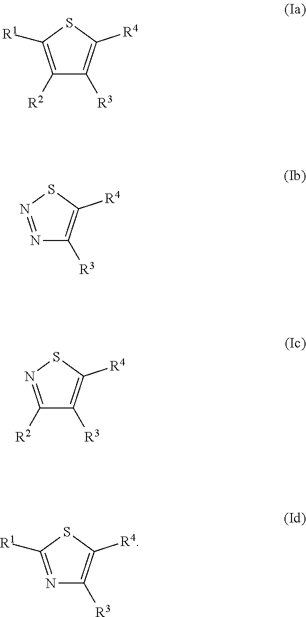

Embodiment 7 provides the compound of any one of Embodiments 1-6, wherein R⁴ is R⁸.

Embodiment 8 provides the compound of any one of Embodiments 1-7, wherein at least one of the following applies:
R⁸ is —NR⁶C(═O)R⁷, R⁶ is H, and R⁷ is CH(CH₂CH₃)Ph;
R⁸ is —NR⁶C(═O)R⁷, R⁶ is H, and R⁷ is CH(CH₂CH₃)(4-F-Ph);
R⁸ is —NR⁶C(═O)NR⁶R⁷, R⁶ is H, and NR⁶R⁷ is 4-phenyl-piperazine-1-yl; and
R⁸ is —NR⁶C(═O)NR⁶R⁷, R⁶ is H, and NR⁶R⁷ is 4-(2-pyridyl)-piperazine-1-yl.

Embodiment 9 provides the compound of any one of Embodiments 1-8, wherein at least one of the following applies:
R³ is —C(═O)OR⁶ and R⁶ is CH₃;
R³ is —C(═O)OR⁶ and R⁶ is CH₂CH₃; and
R³ is —C(═O)NR⁶R⁶, wherein NR⁶R⁶ is NH(CH₂-aryl),
   wherein the aryl is selected from the group consisting of phenyl, 4-fluorophenyl, 4-chlorophenyl, and 4-trifluoromethylphenyl.

Embodiment 10 provides the compound of any one of Embodiments 1-6, wherein one of R¹ and R⁴ is selected from the group consisting of:

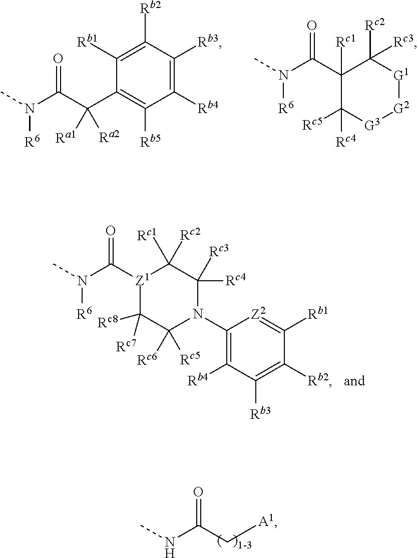

wherein:
R^{a1} and R^{a2}, if present, are each independently selected from the group consisting of H and optionally substituted C₁-C₆ alkyl;
R^{b1}, R^{b2}, R^{b3}, R^{b4}, and R^{b5}, if present, are each independently selected from the group consisting of H, optionally substituted C₁-C₆ alkyl, optionally substituted C₁-C₆ alkoxy, optionally substituted C₁-C₆ haloalkyl, halogen, CN, and NO₂,
   wherein two vicinal substituents selected from the group consisting of R^{b1}, R^{b2}, R^{b3}, R^{b4}, and R^{b5} can combine with the atoms to which they are bound to form an optionally substituted C₂-C₁₀ heterocycloalkyl;
A¹ is selected from the group consisting of optionally substituted C₂-C₁₀ heteroaryl and optionally substituted C₂-C₁₀ heterocycloalkyl;
G¹ is selected from the group consisting of a bond and C(R^{c6})(R^{c7});
G² is selected from the group consisting of a bond and C(R^{c8})(R^{c9});
G³ is selected from the group consisting of a bond and C(R^{c10})(R^{c11});
R^{c1}, R^{c2}, R^{c3}, R^{c4}, R^{c5}, R^{c6}, R^{c7}, R^{c8}, R^{c9}, R^{c10}, and R^{c11}, if present, are each independently selected from the group consisting of H, optionally substituted C₁-C₆ alkyl, optionally substituted C₁-C₆ alkoxy, optionally substituted C₁-C₆ haloalkyl, halogen,
   wherein two vicinal substituents selected from the group consisting of R^{c1}, R^{c2}, R^{c3}, R^{c4}, R^{c5}, R^{c6}, R^{c7}, R^{c8}, R^{c9}, R^{c10}, and R^{c11} can combine with the atoms to which they are bound to form an optionally substituted C₆-C₁₀ aryl;
Z¹ is selected from the group consisting of N and CR^{c9}; and
Z² is selected from the group consisting of N and CR^{b5}.

Embodiment 11 provides the compound of Embodiment 10, wherein one of the following applies:
(a) R^{a1} is H and R^{a2} is ethyl; or
(b) R^{a1} is ethyl and R^{a2} is H.

Embodiment 12 provides the compound of Embodiment 10 or 11, wherein each of R^{b1}, R^{b2}, R^{b3}, R^{b4}, and R^{b5}, if present, are independently selected from the group consisting of H, Me, OMe, F, Cl, CF₃, CN, and NO₂.

Embodiment 13 provides the compound of Embodiment 10, wherein each of R^{c1}, R^{c2}, R^{c3}, R^{c4}, R^{c5}, R^{c6}, R^{c7}, R^{c8}, R^{c9}, R^{c10}, and R^{c11}, if present, are independently selected from the group consisting of H and Ph.

Embodiment 14 provides the compound of Embodiment 10 or 13, wherein at least one of the following applies:
(a) none of G¹, G², and G³ are a bond;
(b) one of G¹, G², and G³ is a bond;
(c) two of G¹, G², and G³ are a bond; and
(d) each of G¹, G², and G³ are a bond.

Embodiment 15 provides the compound of Embodiment 10, wherein A¹ is selected from the group consisting of

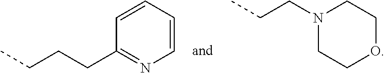

Embodiment 16 provides the compound of any one of Embodiments 10-15, wherein at least one of the following applies:
(a) R² is optionally substituted C₁-C₆ alkyl,
   R³ is C(═O)OR⁶,
   one of R¹ and R⁴ is

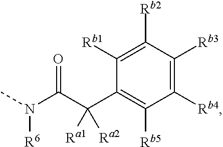

and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(b) R³ is optionally substituted C₁-C₆ alkyl,
   R² is C(═O)OR⁶,
   one of R¹ and R⁴ is

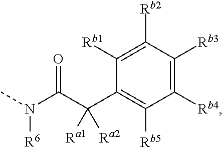

and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(c) R² is optionally substituted C₁-C₆ alkyl,
   R³ is CN,
   one of R¹ and R⁴ is

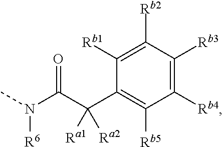

and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(d) R² is C(═O)OR⁶,
   one of R¹ and R⁴ is

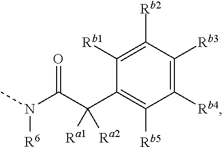

and
no more than one of R³ and R¹ or R⁴ is C₁-C₆ alkyl;
(e) R¹ is H,
   R² is H,
   R³ is —C(═O)NR⁶R⁶, and
   no more than one occurrence of R⁶ is H;
(f) R¹ is

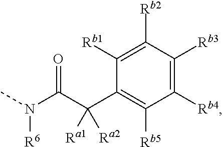

R² is methyl,
R³ is H, and
R⁴ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl);
(g) R⁴ is

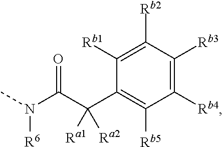

R³ is methyl,
R² is H, and
R¹ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl);
(h) R¹ is C(═O)NH₂,
   R⁴ is

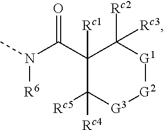

one or less of G¹, G², and G³ is a bond, and
a pair of vicinal substituents selected from the group consisting of R^{c2}, R^{c3}, R^{c4}, R^{c5}, R^{c6}, R^{c7}, R^{c8}, R^{c9}, R^{c10}, and R^{c11} combine with the atoms to which they are bound to form a C₆-C₁₀ aryl;
(i) R¹ is C(═O)NH₂,
   R⁴ is

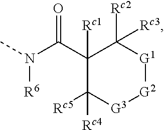

one or less of G¹, G², and G³ is a bond, and
a pair of vicinal substituents selected from the group consisting of R^{c2}, R^{c3}, R^{c4}, R^{c5}, R^{c6}, R^{c7}, R^{c8}, R^{c9}, R^{c10}, and R^{c11} combine with the atoms to which they are bound to form a C₆-C₁₀ aryl;
(j) Y is N, X is CR¹,
   one of R¹ and R⁴ is

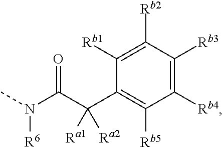

and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(k) X is N, Y is CR²,
   R⁴ is

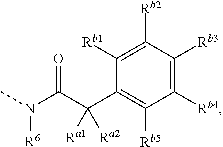

and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(l) X is N,
   Y is N,
   R⁴ is

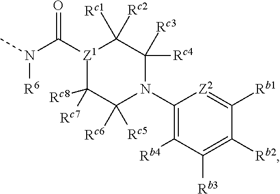

and
Z¹ is CR^{c9}; and
(m) X is N,
   Y is N,
   R⁴ is

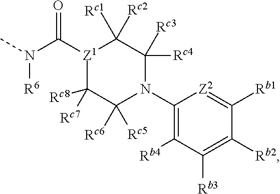

Z² is CR^{b5}, and
at least one selected from the group consisting of R^{b1}, R^{b2}, R^{b3}, R^{b4}, and R^{b5} is selected from the group consisting of halogen, C₁-C₆ alkyl, NO₂, and CN.

Embodiment 17 provides the compound of any one of Embodiments 1-16, wherein R¹ is selected from the group consisting of H, Me, C(═O)NH₂, C(═O)NMe₂, C(═O)NEt₂, C(═O)OEt, C(═O)OMe, C(═O)OEt, C(═O)Ot-Bu, C(═O)O(CH₂)₂NH₂, C(═O)NH(CH₂)₃NH₂,

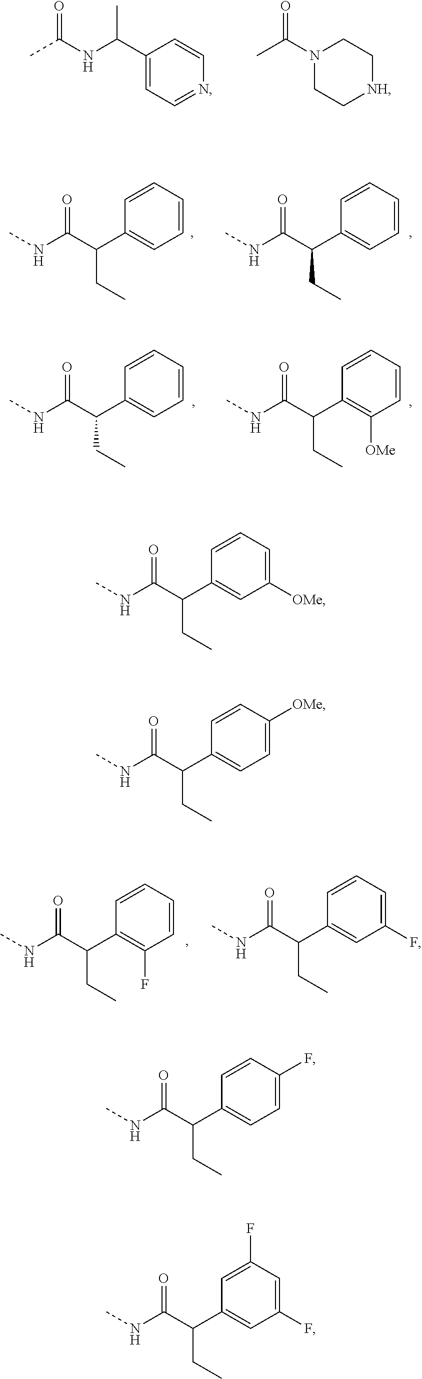

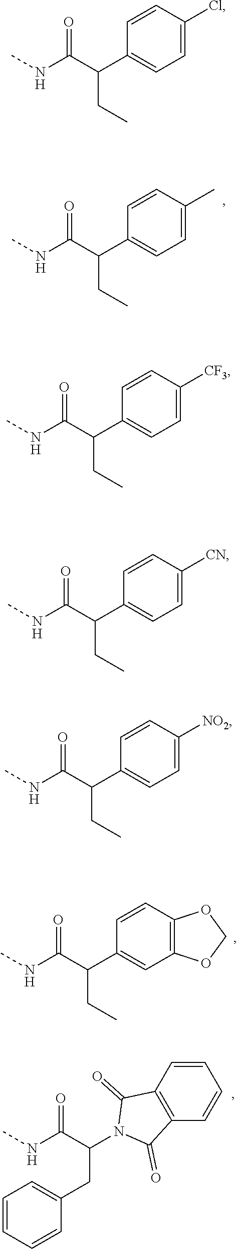

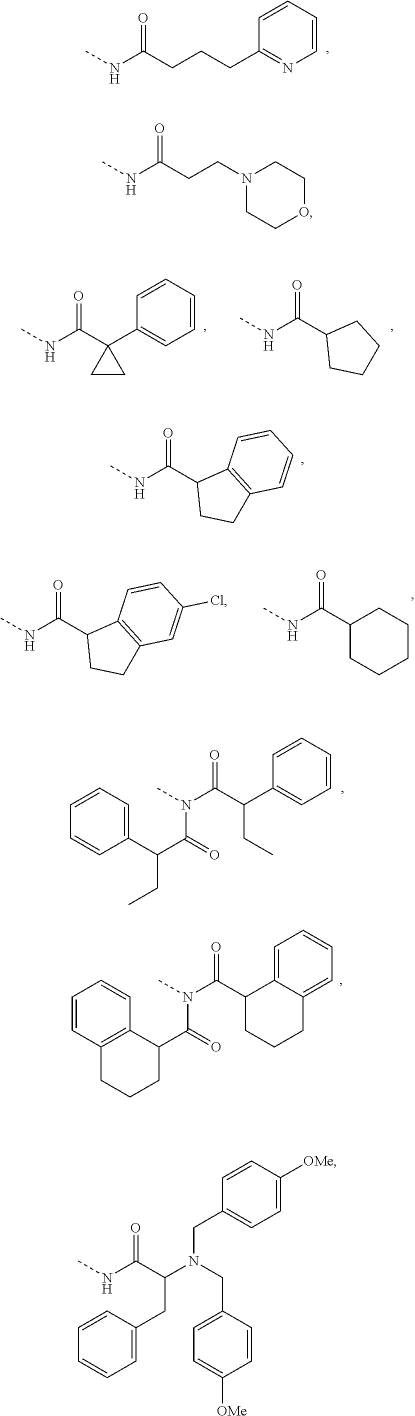

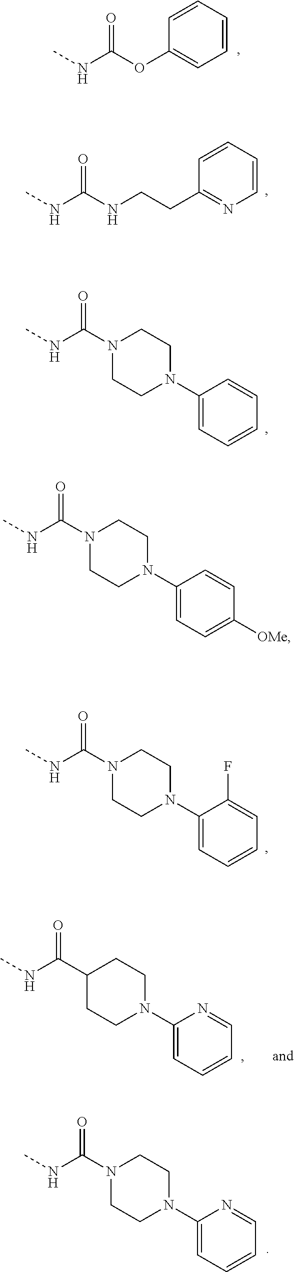

Embodiment 18 provides the compound of any one of Embodiments 1-17, wherein R⁴ is selected from the group consisting of Me, C(═O)NH₂, C(═O)NMe₂, C(═O)NEt₂, C(═O)OEt, C(═O)OMe, C(═O)OEt, C(═O)Ot-Bu, C(═O)O(CH₂)₂NH₂, C(═O)NH(CH₂)₃NH₂,

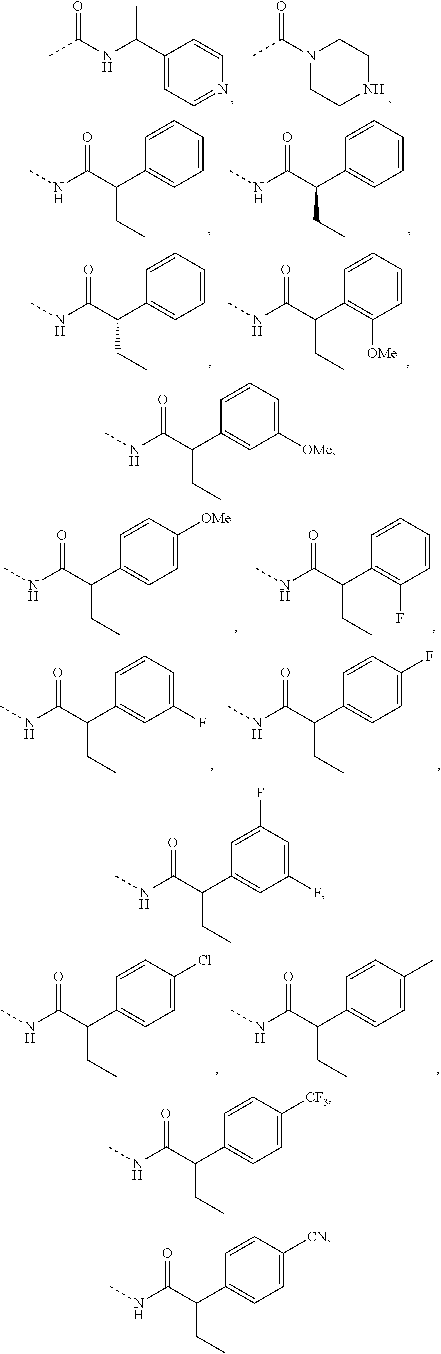

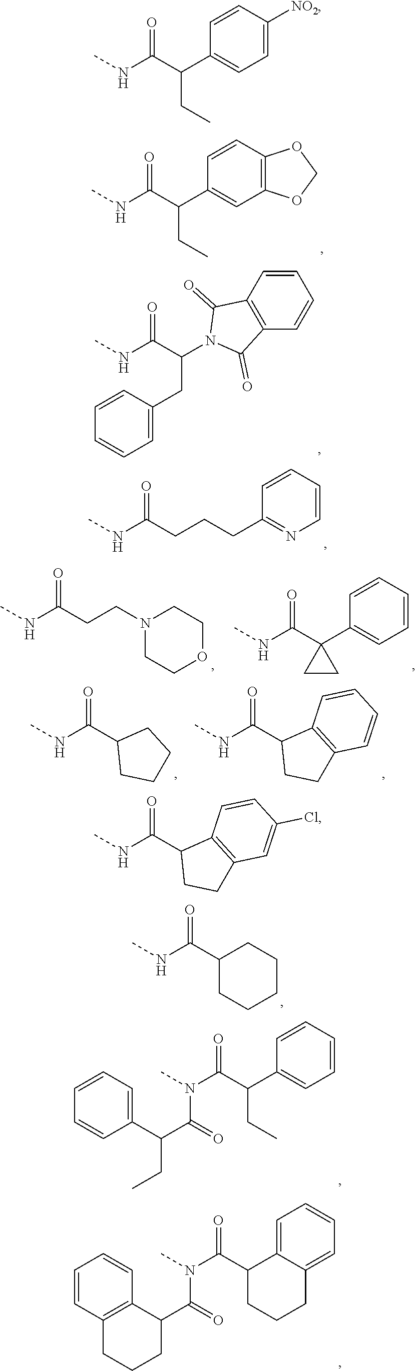

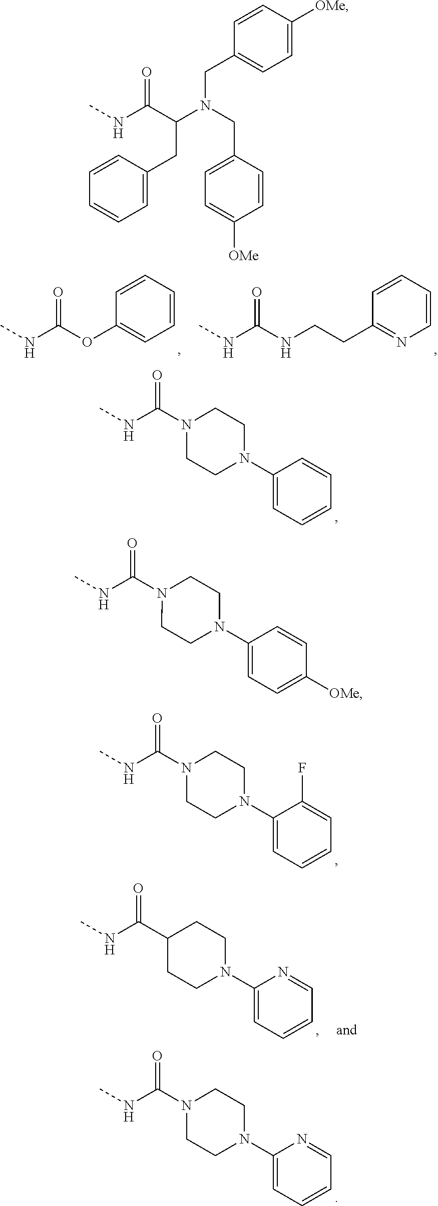

Embodiment 19 provides the compound of any one of Embodiments 1-18, wherein R² is selected from the group consisting of H, Me, and Et.

Embodiment 20 provides the compound of any one of Embodiments 1-19, wherein R³ is selected from the group consisting of H, Me, Et, C(═O)OMe, C(═O)OEt, C(═O)NH₂, CN, Ph, 4-trifluoromethylphenyl, 4-fluorophenyl,

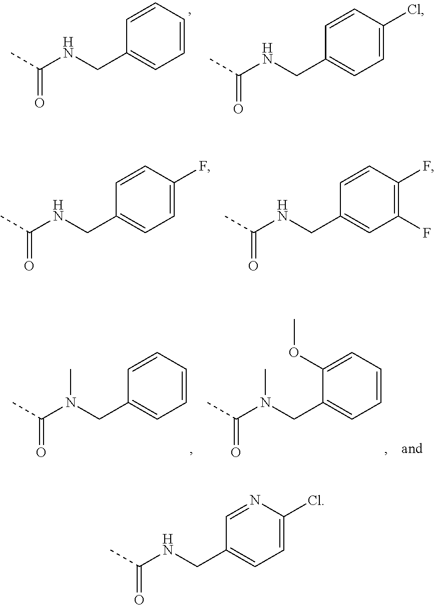

Embodiment 21 provides the compound of any one of Embodiments 1-20, which is selected from the group consisting of Compounds 22, 32, 33, 34, 35, 36, 39, 47, 50, 57, 68, 75, 82, 87, 89, 90, 95, 96, 97, 98, 99, 106, 109, 110, 111, 112, 114, 115, 116, 118, 119, 123, 124, 127, 130, 131, 132, 134, 135, 144, 153, 154, 155, 159, 160, 161, 162, 163, 165, 167, 168, 169, 170, 172, 173, 174, 175, 179, 180, 181, 182, 183, 184, 185, 186, 187, 188, 189, 190, and 191.

Embodiment 22 provides the compound of any one of Embodiments 1-21, wherein each occurrence of alkyl or cycloalkyl is independently optionally substituted with at least one substituent selected from the group consisting of C₁-C₆ alkyl, halo, carbonyl (C═O), —OR, optionally substituted phenyl, optionally substituted heteroaryl, optionally substituted heterocyclyl, —N(R)(R), —N(R)—(C═O)R, —C(═O)R, —C(═O)(optionally substituted phenyl), —C(═O)(optionally substituted heteroaryl), —C(═O)N(R)(R), —C(═O)(CH₂)₀₋₃OR, —S(═O)₂R, and —SO₂N(R)(R), wherein each occurrence of R is independently selected from the group consisting of H, C₁-C₆ alkyl, and C₃-C₈ cycloalkyl.

Embodiment 23 provides the compound of any one of Embodiments 1-22, wherein each occurrence of phenyl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently selected from the group consisting of H, C₁-C₆ alkyl, and C₃-C₈ cycloalkyl.

Embodiment 24 provides the compound of any one of Embodiments 1-23, wherein each occurrence of phenyl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently selected from the group consisting of H, C₁-C₆ alkyl, and C₃-C₈ cycloalkyl.

Embodiment 25 provides a pharmaceutical composition comprising at least one compound of any one of Embodiments 1-24 and at least one pharmaceutically acceptable excipient.

Embodiment 26 provides a pharmaceutical composition comprising at least one compound selected from the group consisting of compounds 1, 2, 4, 5, 8, 9, 15, 16, 17, 19, 24, 26, 29, 31, 40, 66, 72, 76, 77, 78, 83, 84, 86, 99, and 111 and at least one pharmaceutically acceptable excipient.

Embodiment 27 provides the pharmaceutical composition of Embodiment 25 or 26, wherein the at least one compound is compound 111.

Embodiment 28 provides the pharmaceutical composition of Embodiment 27, wherein the at least one pharmaceutically acceptable excipient is at least one selected from the group consisting of water, polyethylene glycol (PEG) 400, PEG 300, propylene glycol (PG), benzyl alcohol, polysorbate 80, diethylene glycol monoethyl ether (DEGEE), isopropyl myristate, ethanol, diisopropyl adipate, C₁₂₋₁₅ alkyl lactate, thickening agent, hydroxypropyl cellulose, and PEG 4000.

Embodiment 29 provides the pharmaceutical composition of Embodiment 28, wherein the thickening agent comprises concentrated dispersion of acrylamide and sodium acryloyldimethyl taurate copolymer in isohexadecane.

Embodiment 30 provides the pharmaceutical composition of Embodiment 29, wherein at least one of the following is present:
(a) compound 111, which comprises about 0.1% to about 10.0% (w/w) of the pharmaceutical composition;
(b) water, which comprises about 10% to about 15% (w/w) of the pharmaceutical composition;
(c) PEG 400, which comprises about 20% to about 40% (w/w) of the pharmaceutical composition;
(d) PEG 300, which comprises about 35% to about 60% (w/w) of the pharmaceutical composition;
(e) PG, which comprises about 5% to about 15% (w/w) of the pharmaceutical composition;
(f) benzyl alcohol, which comprises about 0.1% to about 5% (w/w) of the pharmaceutical composition;
(g) polysorbate 80, which comprises about 1% to about 10% (w/w) of the pharmaceutical composition;
(h) DEGEE, which comprises about 1% to about 15% (w/w) of the pharmaceutical composition;
(i) isopropyl myristate, which comprises about 0.1% to about 5% (w/w) of the pharmaceutical composition;
(j) ethanol, which comprises about 5% to about 15% (w/w) of the pharmaceutical composition;
(k) diisopropyl adipate, which comprises about 5% to about 15% (w/w) of the pharmaceutical composition;
(l) C₁₂₋₁₅ alkyl lactate, which comprises about 5% to about 15% (w/w) of the pharmaceutical composition
(m) thickening agent, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

Embodiment 31 provides the pharmaceutical composition of Embodiment 30, consisting essentially of 111 (about 1.0% w/w), PEG 400 (about 24.3% w/w), PEG 300 (about 40.0% w/w), PG (about 10.0% w/w), benzyl alcohol (about 2.7% w/w), polysorbate 80 (about 5.0% w/w), DEGEE (about 5.0% w/w), isopropyl myristate (about 2.0% w/w), and PEG 4000 (about 10% w/w).

Embodiment 32 provides the pharmaceutical composition of Embodiment 30, consisting essentially of 111 (about 1.0% w/w), PEG 400 (about 23.6% w/w), PEG 300 (about 40.0% w/w), PG (about 10.0% w/w), benzyl alcohol (about 2.7% w/w), polysorbate 80 (about 5.0% w/w), DEGEE (about 5.0% w/w), and PEG 4000 (about 10.0% w/w).

Embodiment 33 provides the pharmaceutical composition of Embodiment 30, consisting essentially of 111 (about 1.0% w/w), PEG 400 (about 34.0% w/w), PEG 300 (about 40.0% w/w), PG (about 10% w/w), benzyl alcohol (about 2.0% w/w), polysorbate 80 (about 5.0% w/w), DEGEE (about 5.0% w/w), isopropyl myristate (about 2.0% w/w), and hydroxypropyl cellulose (about 1.0% w/w).

Embodiment 34 provides the pharmaceutical composition of Embodiment 30, consisting essentially of 111 (about 1.0% w/w), water (about 13.3% w/w), PEG 300 (about 57.0% w/w), PG (about 10% w/w), benzyl alcohol (about 2.7% w/w), polysorbate 80 (about 5.0% w/w) DEGEE (about 5.0% w/w), isopropyl myristate (about 2.0% w/w), and thickening agent (about 4% w/w).

Embodiment 35 provides the pharmaceutical composition of Embodiment 30, consisting essentially of 111 (about 1.0% w/w), PEG 300 (about 48.0% w/w), PG (about 10% w/w), benzyl alcohol (about 1.5% w/w), DEGEE (about 10% w/w), ethanol (about 8.5% w/w), diisopropyl adipate (about 10% w/w), C₁₂₋₁₅ alkyl lactate (about 10% w/w), and hydroxypropyl cellulose (about 1.0% w/w).

Embodiment 36 provides the pharmaceutical composition of Embodiment 25 or 26, wherein the at least one compound is compound 99.

Embodiment 37 provides the pharmaceutical composition of Embodiment 36, wherein the at least one pharmaceutically acceptable excipient is at least one selected from the group consisting of water, PEG 400, PG, benzyl alcohol, polysorbate 80, DEGEE, isopropyl myristate, ethanol, diisopropyl adipate, C₁₂₋₁₅ alkyl lactate, dimethyl isosorbide, PEG 40 hydrogenated castor oil (HCO), hydroxypropyl cellulose, and PEG 4000.

Embodiment 38 provides the pharmaceutical composition of Embodiment 36 or 37, wherein at least one of the following is present:
(a) compound 99, which comprises about 0.1% to about 10.0% (w/w) of the pharmaceutical composition;
(b) water, which comprises about 1% to about 10% (w/w) of the pharmaceutical composition;
(c) PEG 400, which comprises about 25% to about 35% (w/w) of the pharmaceutical composition;
(d) PG, which comprises about 10% to about 30% (w/w) of the pharmaceutical composition;
(e) benzyl alcohol, which comprises about 0.1% to about 5% (w/w) of the pharmaceutical composition;
(f) polysorbate 80, which comprises about 1% to about 10% (w/w) of the pharmaceutical composition;
(g) DEGEE, which comprises about 10% to about 50% (w/w) of the pharmaceutical composition;
(h) isopropyl myristate, which comprises about 0.1% to about 5% (w/w) of the pharmaceutical composition;
(i) ethanol, which comprises about 20% to about 35% (w/w) of the pharmaceutical composition;
(j) diisopropyl adipate, which comprises about 5% to about 15% (w/w) of the pharmaceutical composition;
(k) C₁₂₋₁₅ alkyl lactate, which comprises about 1% to about 15% (w/w) of the pharmaceutical composition;
(l) dimethyl isosorbide, which comprises about 5% to about 15% (w/w) of the pharmaceutical composition;
(m) PEG 40 HCO, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

Embodiment 39 provides the pharmaceutical composition of Embodiment 38, consisting essentially of 99 (about 1.0% w/w), PEG 400 (about 28.3% w/w), PG (about 20.0% w/w), benzyl alcohol (about 2.7% w/w), polysorbate 80 (about 5.0% w/w), DEGEE (about 25.0% w/w), isopropyl myristate (about 2.0% w/w), dimethyl isosorbide (about 10.0% w/w), PEG 40 HCO (about 5.0% w/w), and hydroxypropyl cellulose (about 1.0% w/w).

Embodiment 40 provides the pharmaceutical composition of Embodiment 38, consisting essentially of 99 (about 1.0% w/w), PEG 400 (about 30.30% w/w), PG (about 20.0% w/w), benzyl alcohol (about 2.7% w/w), polysorbate (about 5.0% w/w), DEGEE (about 25.0% w/w), dimethyl isosorbide (about 10.0% w/w), PEG 40 HCO (about 5.0% w/w), and hydroxypropyl cellulose (about 1.0% w/w).

Embodiment 41 provides the pharmaceutical composition of Embodiment 38, consisting essentially of 99 (about 1.0% w/w), PEG 400 (about 19.30% w/w), PG (about 20.0% w/w), benzyl alcohol (about 2.7% w/w), polysorbate (about 5.0% w/w), DEGEE (about 25.0% w/w), isopropyl myristate (about 2.0% w/w), dimethyl isosorbide (about 10.0% w/w), PEG 40 HCO (about 5.0% w/w), and PEG 4000 (about 10.0% w/w).

Embodiment 42 provides the pharmaceutical composition of Embodiment 38, consisting essentially of 99 (about 1.0% w/w), DEGEE (about 40.0% w/w), ethanol (about 28.0% w/w), diisopropyl adipate (about 10.0% w/w), C₁₂₋₁₅ alkyl lactate (about 10.0% w/w), dimethyl isosorbide (about 10.0% w/w), and hydroxypropyl cellulose (about 1.0% w/w).

Embodiment 43 provides the pharmaceutical composition of Embodiment 38, consisting essentially of 99 (about 1.0% w/w), water (about 5.0% w/w), DEGEE (about 42.5% w/w), ethanol (about 25.0% w/w), diisopropyl adipate (about 10.0% w/w), C₁₂₋₁₅ alkyl lactate (about 5.0% w/w), dimethyl isosorbide (about 10.0% w/w), and hydroxypropyl cellulose (about 1.5% w/w).

Embodiment 44 provides the pharmaceutical composition of any one of Embodiments 25-43, wherein the pharmaceutical composition is formulated for topical administration.

Embodiment 45 provides the pharmaceutical composition of Embodiment 44, wherein the topical formulation comprises a gel or ointment.

Embodiment 46 provides a method of treating, ameliorating, and/or preventing an orthopoxvirus infection in a human subject in need thereof, wherein the method comprises administering to the subject a therapeutically effective amount of at least one pharmaceutical composition of any of Embodiments 25-45, and/or a compound of formula (II):

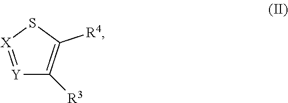

wherein:
X is CR¹ or N;
Y is CR² or N;
R¹ is H, optionally substituted C₁-C₆ alkyl, —C(═O)NR⁶R⁶, —C(═O)OR⁶, or R⁸;
R² is H or optionally substituted C₁-C₆ alkyl;
R³ is H, —CN, —C(═O)OR⁶, —C(═O)NR⁶R⁶, optionally substituted phenyl, or optionally substituted C₁-C₆ alkyl;
R⁴ is —C(═O)OR⁶ or R⁸;
each occurrence of R⁵ is independently optionally substituted C₁-C₆ alkyl or optionally substituted phenyl;
each occurrence of R⁶ is independently H or optionally substituted C₁-C₆ alkyl,
   or two R⁶ combine with the N atom to which both are bound to form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷;
each occurrence of R⁷ is independently optionally substituted C₁-C₆ alkyl, optionally substituted cycloalkyl, CH (optionally substituted heterocyclyl)(R⁵), CH(R⁵)(R⁵), or optionally substituted 4-7 membered heterocyclyl,
   or R⁶ and R⁷ combine with the N atom to which both are bound to form optionally substituted 4-7 membered heterocyclyl;
with the proviso that (I) comprises a single R⁸;
   or a salt, solvate, enantiomer, diastereoisomer, geometric isomer, or tautomer thereof.

Embodiment 47 provides the method of Embodiment 46, wherein the orthopoxvirus infection is caused by a virus selected from the group consisting of Molluscum contagiosum virus (MCV), amelpox virus, cowpox virus, mousepox virus, horsepox virus, monkeypox virus, raccoonpox virus, tanapox virus, varioloa (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus).

Embodiment 48 provides the method of Embodiment 47, wherein the orthopoxvirus infection is caused by a Molluscum contagiosum virus (MCV).

Embodiment 49 provides the method of Embodiment 46, wherein the compound or composition is applied to the skin of the subject.

Embodiment 50 provides the method of Embodiment 46, wherein the compound or composition is applied to at least one MCV lesion on the skin of the subject.

Embodiment 51 provides the method of Embodiment 46, wherein the at least one compound or composition is formulated as a topical pharmaceutical composition.

Embodiment 52 provides the method of Embodiment 51, wherein the topical pharmaceutical composition comprises a gel or ointment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, enantiomer, diastereoisomer, geometric isomer, or tautomer thereof: 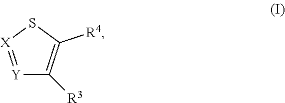
wherein:
X is CR¹ or N;
Y is CR² or N;
optionally wherein one of the following applies: X is CR¹ and Y is CR²; X is N and Y is N; X is CR¹ and Y is N; X is N and Y is CR²
R¹ is H, optionally substituted C₁-C₆ alkyl, —C(═O)NR⁶R⁶, —C(═O)OR⁶, or R⁸;
R² is H or optionally substituted C₁-C₆ alkyl;
R³ is H, —CN, —C(═O)OR⁶, —C(═O)NR⁶R⁶, optionally substituted phenyl, or optionally substituted C₁-C₆ alkyl;
R⁴ is —C(═O)OR⁶ or R⁸;
each occurrence of R⁵ is independently optionally substituted C₁-C₆ alkyl or optionally substituted phenyl;
each occurrence of R⁶ is independently H or optionally substituted C₁-C₆ alkyl,
or two R⁶ combine with the N atom to which both are bound to form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷;
each occurrence of R⁷ is independently optionally substituted C₁-C₆ alkyl, optionally substituted cycloalkyl, CH (optionally substituted heterocyclyl)(R⁵), CH(R⁵)(R⁵), or optionally substituted 4-7 membered heterocyclyl,
or R⁶ and R⁷ combine with the N atom to which both are bound to form optionally substituted 4-7 membered heterocyclyl;
with the proviso that (I) comprises a single R⁸; and
with the proviso that the compound is not selected from the group consisting of Compounds 1, 2, 4, 5, 8, 9, 15, 16, 17, 19, 24, 26, 29, 31, 40, 66, 72, 76, 77, 78, 83, 84, and 86.

2-5. (canceled)

6. The compound of claim 1, which is selected from the group consisting of:

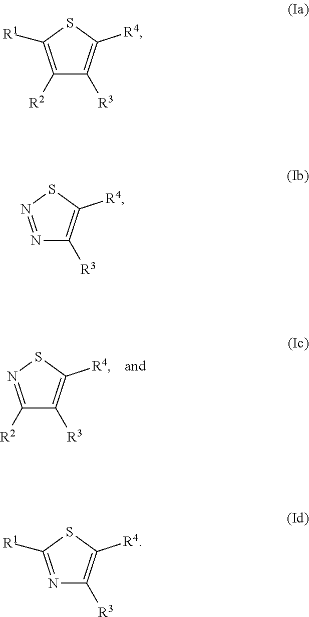

7. The compound of claim 1, wherein R⁴ is R⁵.

8. The compound of claim 1, wherein at least one of the following applies:
R⁸ is —NR⁶C(═O)R⁷, R⁶ is H, and R⁷ is CH(CH₂CH₃)Ph;
R⁸ is —NR⁶C(═O)R⁷, R⁶ is H, and R⁷ is CH(CH₂CH₃)(4-F-Ph);
R⁸ is —NR⁶C(═O)NR⁶R⁷, R⁶ is H, and NR⁶R⁷ is 4-phenyl-piperazine-1-yl; and
R⁸ is —NR⁶C(═O)NR⁶R⁷, R⁶ is H, and NR⁶R⁷ is 4-(2-pyridyl)-piperazine-1-yl.

9. The compound of claim 1, wherein at least one of the following applies:
R³ is —C(═O)OR⁶ and R⁶ is CH₃;
R³ is —C(═O)OR⁶ and R⁶ is CH₂CH₃; and
R³ is —C(═O)NR⁶R⁶, wherein NR⁶R⁶ is NH(CH₂-aryl),
wherein the aryl is selected from the group consisting of phenyl, 4-fluorophenyl, 4-chlorophenyl, and 4-trifluoromethylphenyl.

10. The compound of claim 1, wherein one of R¹ and R⁴ is selected from the group consisting of: 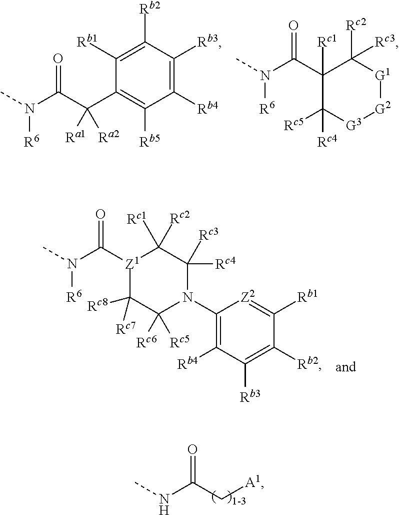
wherein:
R^{a1} and R^{a2}, if present, are each independently selected from the group consisting of H and optionally substituted C₁-C₆ alkyl;
R^{b1}, R^{b2}, R^{b3}, R^{b4}, and R^{b5}, if present, are each independently selected from the group consisting of H, optionally substituted C₁-C₆ alkyl, optionally substituted C₁-C₆ alkoxy, optionally substituted C₁-C₆ haloalkyl, halogen, CN, and NO₂,
wherein two vicinal substituents selected from the group consisting of R^{b1}, R^{b2}, R^{b3}, R^{b4}, and R^{b5} can combine with the atoms to which they are bound to form an optionally substituted C₂-C₁₀ heterocycloalkyl;
A¹ is selected from the group consisting of optionally substituted C₂-C₁₀ heteroaryl and optionally substituted C₂-C₁₀ heterocycloalkyl;
G¹ is selected from the group consisting of a bond and C(R^{c6})(R^{c7});
G² is selected from the group consisting of a bond and C(R^{c8})(R^{c9});
G³ is selected from the group consisting of a bond and C(R^{c10})(R^{c11});
R^{c1}, R^{c2}, R^{c3}, R^{c4}, R^{c5}, R^{c6}, R^{c7}, R^{c8}, R^{c9}, R^{c10}, and R^{c11}, if present, are each independently selected from the group consisting of H, optionally substituted C₁-C₆ alkyl, optionally substituted C₁-C₆ alkoxy, optionally substituted C₁-C₆ haloalkyl, halogen,
wherein two vicinal substituents selected from the group consisting of R^{c1}, R^{c2}, R^{c3}, R^{c4}, R^{c5}, R^{c6}, R^{c7}, R^{c8}, R^{c9}, R^{c10}, and R^{c11} can combine with the atoms to which they are bound to form an optionally substituted C₆-C₁₀ aryl;
Z¹ is selected from the group consisting of N and CR^{c9}; and
Z² is selected from the group consisting of N and CR^{b5}.

11. The compound of claim 10, wherein at least one of the following applies:
(a) R^{a1} is H and R^{a2} is ethyl or R^{a1} is ethyl and R^{a2} is H;
(b) R^{b1}, R^{b2}, R^{b3}, R^{b4}, and R^{b5}, if present, are independently selected from the group consisting of H, Me, OMe, F, Cl, CF₃, CN, and NO₂;
(c) R^{c1}, R^{c2}, R^{c3}, R^{c4}, R^{c5}, R^{c6}, R^{c7}, R^{c8}, R^{c9}, R^{c10}, and R^{c11}, if present, are independently selected from the group consisting of H and Ph;
(d) none of G¹, G², and G³ are a bond, one of G¹, G², and G³ is a bond, two of G¹, G², and G³ are a bond, or each of G¹, G², and G³ are a bond; and
(e) A¹ is selected from the group consisting of

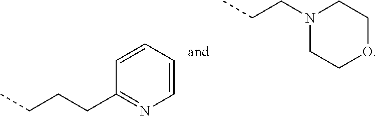

12-15. (canceled)

16. The compound of claim 10, wherein at least one of the following applies:
(a) R² is optionally substituted C₁-C₆ alkyl,
R³ is C(═O)OR⁶,
one of R¹ and R⁴ is 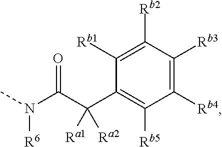
and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(b) R³ is optionally substituted C₁-C₆ alkyl,
R² is C(═O)OR⁶,
one of R¹ and R⁴ is 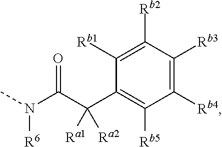
and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(c) R² is optionally substituted C₁-C₆ alkyl,
R³ is CN,
one of R¹ and R⁴ is 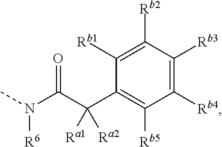
and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(d) R² is C(═O)OR⁶,
one of R¹ and R⁴ is 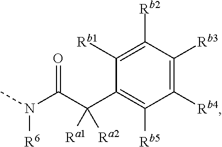
and
no more than one of R³ and R¹ or R⁴ is C₁-C₆ alkyl;
(e) R¹ is H,
R² is H,
R³ is —C(═O)NR⁶R⁶, and
no more than one occurrence of R⁶ is H;
(f) R¹ is 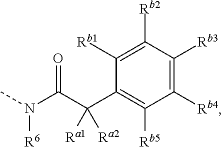
R² is methyl,
R³ is H, and
R⁴ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl);
(g) R⁴ is 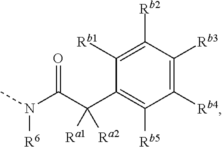
R³ is methyl,
R² is H, and
R¹ is selected from the group consisting of C(═O)O(C₁ alkyl), C(═O)O (optionally substituted C₃ alkyl), C(═O)O (optionally substituted C₄ alkyl), C(═O)O (optionally substituted C₅ alkyl), and C(═O)O (optionally substituted C₆ alkyl);
(h) R¹ is C(═O)NH₂,
R⁴ is 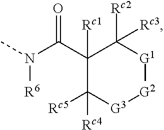
one or less of G¹, G², and G³ is a bond, and
a pair of vicinal substituents selected from the group consisting of R^{c2}, R^{c3}, R^{c4}, R^{c5}, R^{c6}, R^{c7}, R^{c8}, R^{c9}, R^{c10}, and R^{c11} combine with the atoms to which they are bound to form a C₆-C₁₀ aryl;
(i) R¹ is C(═O)NH₂,
R⁴ is 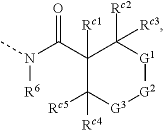
one or less of G¹, G², and G³ is a bond, and
a pair of vicinal substituents selected from the group consisting of R^{c2}, R^{c3}, R^{c4}, R^{c5}, R^{c6}, R^{c7}, R^{c8}, R^{c9}, R^{c10}, and R^{c11} combine with the atoms to which they are bound to form a C₆-C₁₀ aryl;
(j) Y is N, X is CR¹,
one of R¹ and R⁴ is 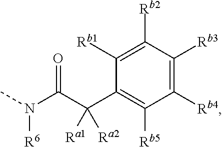
and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(k) X is N, Y is CR²,
R⁴ is 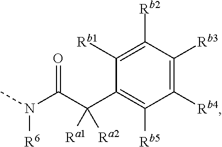
and
at least one selected from the group consisting of R^{b1}, R^{b2}, R^{b3}, R^{b4}, and R^{b5} is selected from the group consisting of Me, OMe, F, Cl, CF₃, CN, and NO₂, or two vicinal substituents selected from the group consisting of R^{b1}, R^{b2}, R^{b3}, R^{b4}, and R^{b5} combine to form a methylenedioxy;
(l) X is N,
Y is N,
R⁴ is 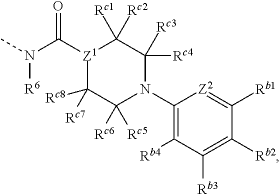
and
Z¹ is CR^{c9}; and
(m) X is N,
Y is N,
R⁴ is 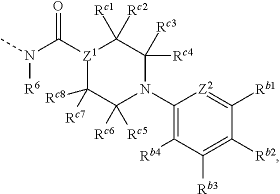
Z² is CR^{b5}, and
at least one selected from the group consisting of R^{b1}, R^{b2}, R^{b3}, R^{b4}, and R^{b5} is selected from the group consisting of halogen, C₁-C₆ alkyl, NO₂, and CN.

17. The compound of claim 1, wherein at least one of the following applies:
(a) R¹ is selected from the group consisting of H, Me, C(═O)NH₂, C(═O)NMe₂, C(═O)NEt₂, C(═O)OEt, C(═O)OMe, C(═O)OEt, C(═O)Ot-Bu, C(═O)O(CH₂)₂NH₂, C(═O)NH(CH₂)₃NH₂, 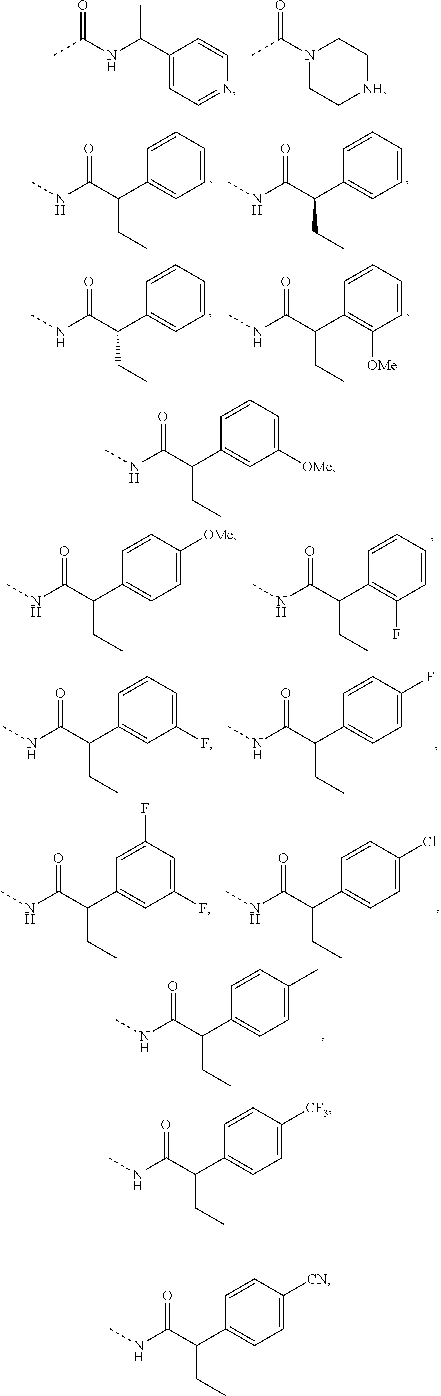 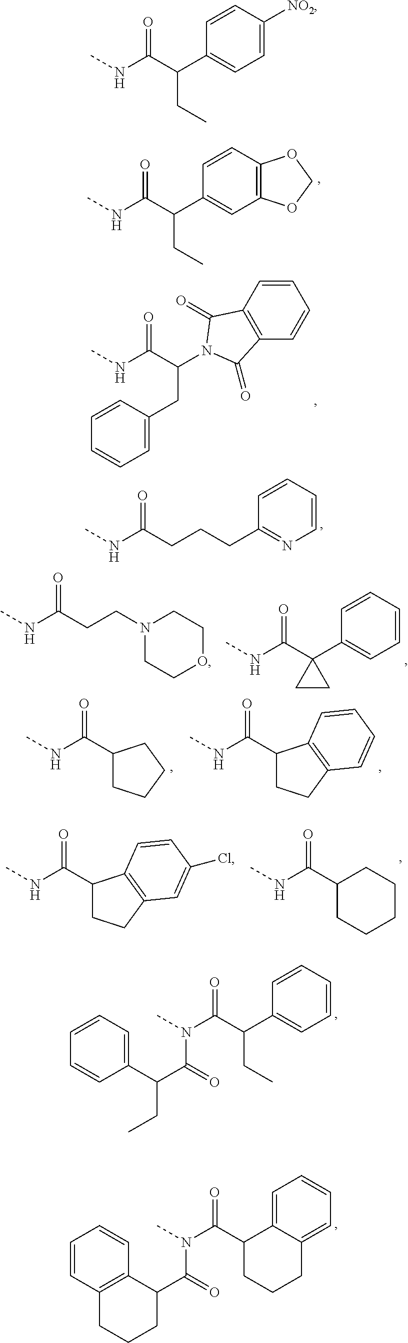 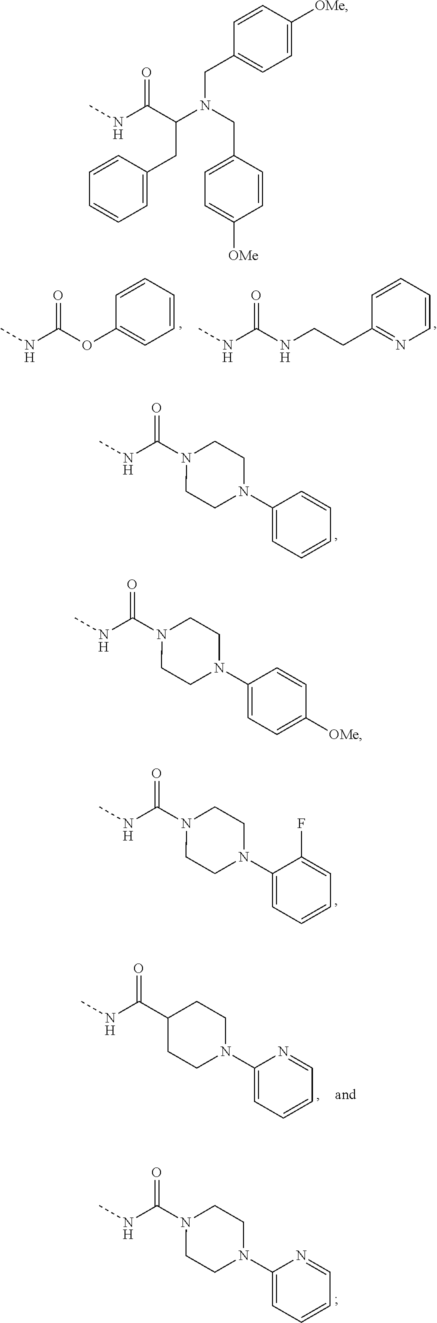
(b) R² is selected from the group consisting of H, Me, and Et;
(c) R³ is selected from the group consisting of H, Me, Et, C(═O)OMe, C(═O)OEt, C(═O)NH₂, CN, Ph, 4-trifluoromethyl phenyl, 4-fluorophenyl, 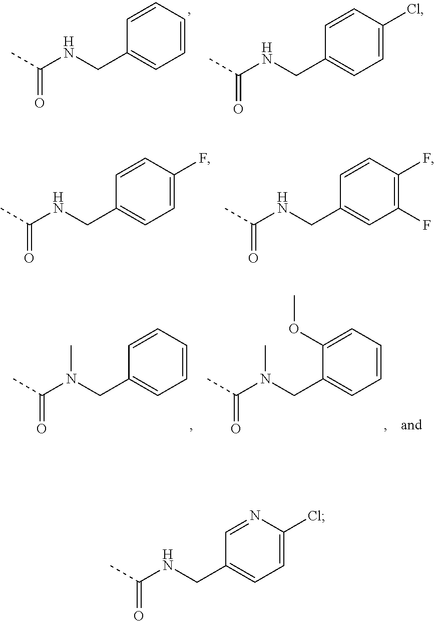
and
(d) R⁴ is selected from the group consisting of Me, C(═O)NH₂, C(═O)NMe₂, C(═O)NEt₂, C(═O)OEt, C(═O)OMe, C(═O)OEt, C(═O)Ot-Bu, C(═O)O(CH₂)₂NH₂, C(═O)NH(CH₂)₃NH₂,

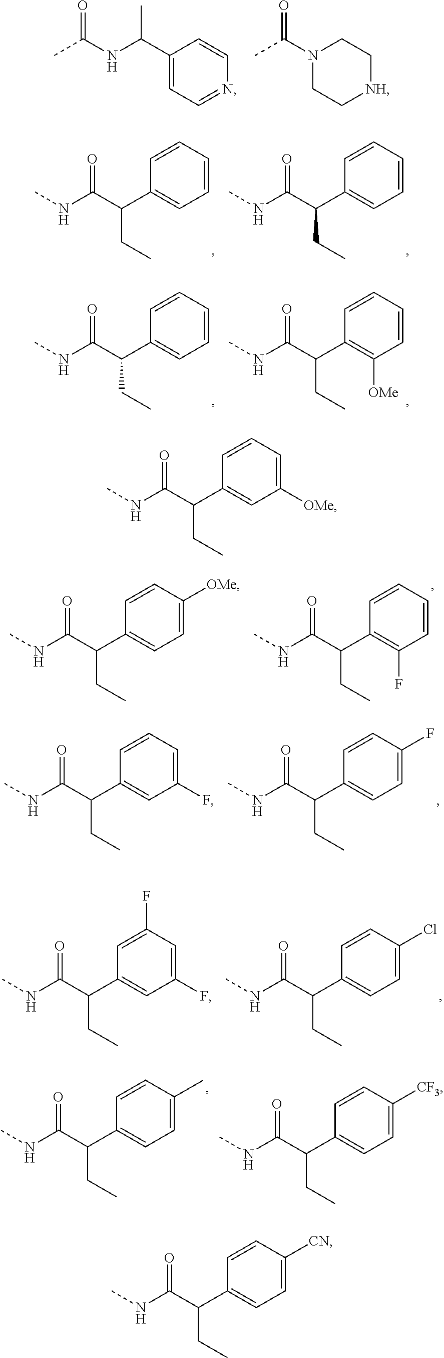

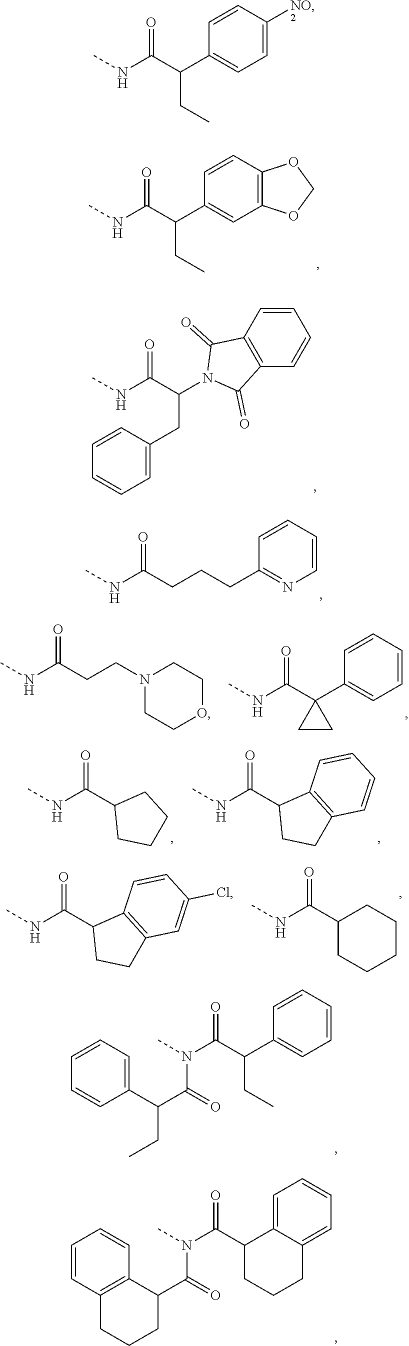

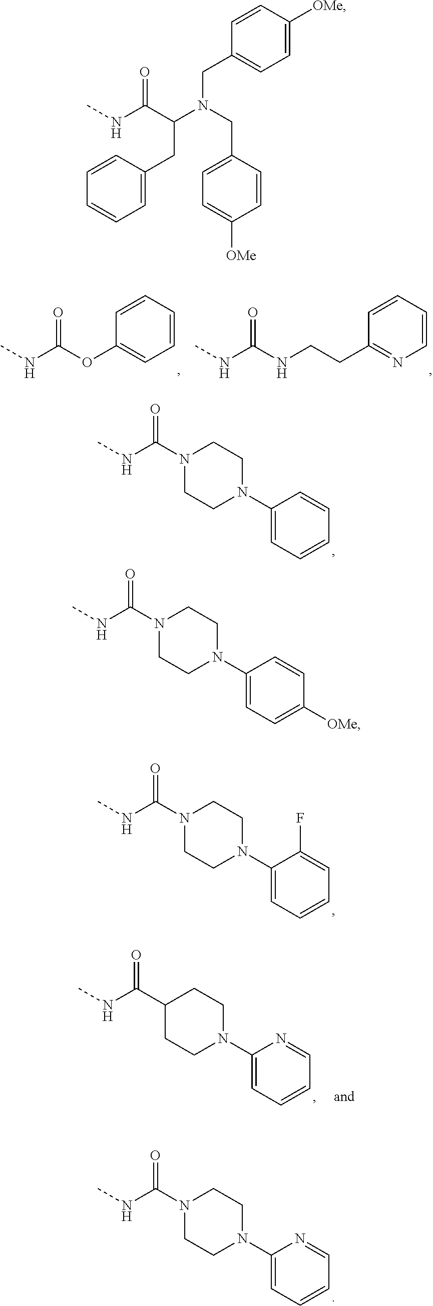

18-20. (canceled)

21. The compound of claim 1, which is selected from the group consisting of Compounds 22, 32, 33, 34, 35, 36, 39, 47, 50, 57, 68, 75, 82, 87, 89, 90, 95, 96, 97, 98, 99, 106, 109, 110, 111, 112, 114, 115, 116, 118, 119, 123, 124, 127, 130, 131, 132, 134, 135, 144, 153, 154, 155, 159, 160, 161, 162, 163, 165, 167, 168, 169, 170, 172, 173, 174, 175, 179, 180, 181, 182, 183, 184, 185, 186, 187, 188, 189, 190, and 191.

22. The compound of claim 1, wherein at least one of the following applies:
(a) each occurrence of alkyl or cycloalkyl is independently optionally substituted with at least one substituent selected from the group consisting of C₁-C₆ alkyl, halo, carbonyl (C═O), —OR, optionally substituted phenyl, optionally substituted heteroaryl, optionally substituted heterocyclyl, —N(R)(R), —N(R)—(C═O)R, —C(═O)R, —C(═O)(optionally substituted phenyl), —C(═O)(optionally substituted heteroaryl), —C(═O)N(R)(R), —C(═O)(CH₂)₀₋₃OR, —S(═O)₂R, and —SO₂N(R)(R), wherein each occurrence of R is independently selected from the group consisting of H, C₁-C₆ alkyl, and C₃-C₈ cycloalkyl; and
(b) each occurrence of phenyl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently selected from the group consisting of H, C₁-C₆ alkyl, and C₃-C₈ cycloalkyl.

23-24. (canceled)

25. A pharmaceutical composition comprising at least one compound of claim 1 and at least one pharmaceutically acceptable excipient, optionally wherein the compound is selected from the group consisting of compound 1, 2, 4, 5, 8, 9, 15, 16, 17, 19, 24, 26, 29, 31, 40, 66, 72, 76, 77, 78, 83, 84, 86, 99, and 111.

26-27. (canceled)

28. The pharmaceutical composition of claim 25, wherein the at least one pharmaceutically acceptable excipient is at least one selected from the group consisting of water, polyethylene glycol (PEG) 400, PEG 300, propylene glycol (PG), benzyl alcohol, polysorbate 80, diethylene glycol monoethyl ether (DEGEE), isopropyl myristate, ethanol, diisopropyl adipate, C₁₂₋₁₅ alkyl lactate, thickening agent, hydroxypropyl cellulose, and PEG 4000, optionally wherein the thickening agent comprises concentrated dispersion of acrylamide and sodium acryloyldimethyl taurate copolymer in isohexadecane.

29. (canceled)

30. The pharmaceutical composition of claim 28, wherein at least one of the following is present:
(a) compound 111, which comprises about 0.1% to about 10.0% (w/w) of the pharmaceutical composition;
(b) water, which comprises about 10% to about 15% (w/w) of the pharmaceutical composition;
(c) PEG 400, which comprises about 20% to about 40% (w/w) of the pharmaceutical composition;
(d) PEG 300, which comprises about 35% to about 60% (w/w) of the pharmaceutical composition;
(e) PG, which comprises about 5% to about 15% (w/w) of the pharmaceutical composition;
(f) benzyl alcohol, which comprises about 0.1% to about 5% (w/w) of the pharmaceutical composition;
(g) polysorbate 80, which comprises about 1% to about 10% (w/w) of the pharmaceutical composition;
(h) DEGEE, which comprises about 1% to about 15% (w/w) of the pharmaceutical composition;
(i) isopropyl myristate, which comprises about 0.1% to about 5% (w/w) of the pharmaceutical composition;
(j) ethanol, which comprises about 5% to about 15% (w/w) of the pharmaceutical composition;
(k) diisopropyl adipate, which comprises about 5% to about 15% (w/w) of the pharmaceutical composition;
(l) C₁₂₋₁₅ alkyl lactate, which comprises about 5% to about 15% (w/w) of the pharmaceutical composition (m) thickening agent, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

31. The pharmaceutical composition of claim 30, one of the following applies:
(a) the composition consists essentially of 111 (about 1.0% w/w), PEG 400 (about 24.3% w/w), PEG 300 (about 40.0% w/w), PG (about 10.0% w/w), benzyl alcohol (about 2.7% w/w), polysorbate 80 (about 5.0% w/w), DEGEE (about 5.0% w/w), isopropyl myristate (about 2.0% w/w), and PEG 4000 (about 10% w/w);
(b) the composition consists essentially of 111 (about 1.0% w/w), PEG 400 (about 23.6% w/w), PEG 300 (about 40.0% w/w), PG (about 10.0% w/w), benzyl alcohol (about 2.7% w/w), polysorbate 80 (about 5.0% w/w), DEGEE (about 5.0% w/w), and PEG 4000 (about 10.0% w/w);
(c) the composition consists essentially of 111 (about 1.0% w/w), PEG 400 (about 34.0% w/w), PEG 300 (about 40.0% w/w), PG (about 10% w/w), benzyl alcohol (about 2.0% w/w), polysorbate 80 (about 5.0% w/w), DEGEE (about 5.0% w/w), isopropyl myristate (about 2.0% w/w), and hydroxypropyl cellulose (about 1.0% w/w);
(d) the composition consists essentially of 111 (about 1.0% w/w), water (about 13.3% w/w), PEG 300 (about 57.0% w/w), PG (about 10% w/w), benzyl alcohol (about 2.7% w/w), polysorbate 80 (about 5.0% w/w) DEGEE (about 5.0% w/w), isopropyl myristate (about 2.0% w/w), and thickening agent (about 4% w/w); and
(e) the composition consists essentially of 111 (about 1.0% w/w), PEG 300 (about 48.0% w/w), PG (about 10% w/w), benzyl alcohol (about 1.5% w/w), DEGEE (about 10% w/w), ethanol (about 8.5% w/w), diisopropyl adipate (about 10% w/w), C₁₂₋₁₅ alkyl lactate (about 10% w/w), and hydroxypropyl cellulose (about 1.0% w/w).

32-37. (canceled)

38. The pharmaceutical composition of claim 25, wherein at least one of the following is present:
(a) compound 99, which comprises about 0.1% to about 10.0% (w/w) of the pharmaceutical composition;
(b) water, which comprises about 1% to about 10% (w/w) of the pharmaceutical composition;
(c) PEG 400, which comprises about 25% to about 35% (w/w) of the pharmaceutical composition;
(d) PG, which comprises about 10% to about 30% (w/w) of the pharmaceutical composition;
(e) benzyl alcohol, which comprises about 0.1% to about 5% (w/w) of the pharmaceutical composition;
(f) polysorbate 80, which comprises about 1% to about 10% (w/w) of the pharmaceutical composition;
(g) DEGEE, which comprises about 10% to about 50% (w/w) of the pharmaceutical composition;
(h) isopropyl myristate, which comprises about 0.1% to about 5% (w/w) of the pharmaceutical composition;
(i) ethanol, which comprises about 20% to about 35% (w/w) of the pharmaceutical composition;
(j) diisopropyl adipate, which comprises about 5% to about 15% (w/w) of the pharmaceutical composition;
(k) C₁₂₋₁₅ alkyl lactate, which comprises about 1% to about 15% (w/w) of the pharmaceutical composition;
(l) dimethyl isosorbide, which comprises about 5% to about 15% (w/w) of the pharmaceutical composition;
(m) PEG 40 HCO, which comprises about 1% to about 10% (w/w) of the pharmaceutical composition;
(n) hydroxypropyl cellulose, which comprises about 0.1% to about 5% (w/w) of the pharmaceutical composition; and
(o) PEG 4000, which comprises about 5% to about 15% (w/w) of the pharmaceutical composition.

39. The pharmaceutical composition of claim 38, wherein at least one of the following applies:
(a) the composition consists essentially of 99 (about 1.0% w/w), PEG 400 (about 28.3% w/w), PG (about 20.0% w/w), benzyl alcohol (about 2.7% w/w), polysorbate 80 (about 5.0% w/w), DEGEE (about 25.0% w/w), isopropyl myristate (about 2.0% w/w), dimethyl isosorbide (about 10.0% w/w), PEG 40 HCO (about 5.0% w/w), and hydroxypropyl cellulose (about 1.0% w/w);
(b) the composition consists essentially of 99 (about 1.0% w/w), PEG 400 (about 30.30% w/w), PG (about 20.0% w/w), benzyl alcohol (about 2.7% w/w), polysorbate (about 5.0% w/w), DEGEE (about 25.0% w/w), dimethyl isosorbide (about 10.0% w/w), PEG 40 HCO (about 5.0% w/w), and hydroxypropyl cellulose (about 1.0% w/w);
(c) the composition consists essentially of 99 (about 1.0% w/w), PEG 400 (about 19.30% w/w), PG (about 20.0% w/w), benzyl alcohol (about 2.7% w/w), polysorbate (about 5.0% w/w), DEGEE (about 25.0% w/w), isopropyl myristate (about 2.0% w/w), dimethyl isosorbide (about 10.0% w/w), PEG 40 HCO (about 5.0% w/w), and PEG 4000 (about 10.0% w/w);
(d) the composition consists essentially of 99 (about 1.0% w/w), DEGEE (about 40.0% w/w), ethanol (about 28.0% w/w), diisopropyl adipate (about 10.0% w/w), C₁₂₋₁₅ alkyl lactate (about 10.0% w/w), dimethyl isosorbide (about 10.0% w/w), and hydroxypropyl cellulose (about 1.0% w/w); and
(e) the composition consists essentially of 99 (about 1.0% w/w), water (about 5.0% w/w), DEGEE (about 42.5% w/w), ethanol (about 25.0% w/w), diisopropyl adipate (about 10.0% w/w), C₁₂₋₁₅ alkyl lactate (about 5.0% w/w), dimethyl isosorbide (about 10.0% w/w), and hydroxypropyl cellulose (about 1.5% w/w).

40-43. (canceled)

44. The pharmaceutical composition of claim 25, wherein the pharmaceutical composition is formulated for topical administration, optionally wherein the topical formulation comprises a gel or ointment.

45. (canceled)

46. A method of treating, ameliorating, and/or preventing an orthopoxvirus infection in a human subject in need thereof, wherein the method comprises administering to the subject a therapeutically effective amount of at least one pharmaceutical composition of claim 25, and/or a compound of formula (II): 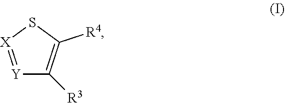
wherein:
X is CR¹ or N;
Y is CR² or N;
R¹ is H, optionally substituted C₁-C₆ alkyl, —C(═O)NR⁶R⁶, —C(═O)OR⁶, or R⁸;
R² is H or optionally substituted C₁-C₆ alkyl;
R³ is H, —CN, —C(═O)OR⁶, —C(═O)NR⁶R⁶, optionally substituted phenyl, or optionally substituted C₁-C₆ alkyl;
R⁴ is —C(═O)OR⁶ or R⁸;
each occurrence of R⁵ is independently optionally substituted C₁-C₆ alkyl or optionally substituted phenyl;
each occurrence of R⁶ is independently H or optionally substituted C₁-C₆ alkyl,
or two R⁶ combine with the N atom to which both are bound to form optionally substituted 4-7 membered heterocyclyl;
R⁸ is —C(═O)NH (optionally substituted acyl), —N (optionally substituted acyl)C(═O)R⁷, —NR⁶C(═O)R⁷ or —NR⁶C(═O)NR⁶R⁷;
each occurrence of R⁷ is independently optionally substituted C₁-C₆ alkyl, optionally substituted cycloalkyl, CH (optionally substituted heterocyclyl)(R⁵), CH(R⁵)(R⁵), or optionally substituted 4-7 membered heterocyclyl,
or R⁶ and R⁷ combine with the N atom to which both are bound to form optionally substituted 4-7 membered heterocyclyl;
with the proviso that (I) comprises a single R⁸;
or a salt, solvate, enantiomer, diastereoisomer, geometric isomer, or tautomer thereof.

47. The method of claim 46, wherein at least one of the following applies:
(a) the orthopoxvirus infection is caused by a virus selected from the group consisting of Molluscum contagiosum virus (MCV), amelpox virus, cowpox virus, mousepox virus, horsepox virus, monkeypox virus, raccoonpox virus, tanapox virus, varioloa (smallpox) virus, Yoka poxvirus, cervidpoxvirus (deerpox), avipoxvirus (fowlpox), capripoxvirus (goatpox), leporipoxvirus (myxoma virus), parapoxvirus (orf virus), suipoxvirus (swinepox), and yatapoxvirus (Yaba-like disease virus),
(b) the compound or composition thereof is applied to at least one MCV lesion on the skin of the subject; and
(c) the at least one compound or composition thereof is formulated as a topical pharmaceutical composition, optionally wherein the topical pharmaceutical composition comprises a gel or ointment.

48-52. (canceled)


SEQUENCE LISTING (2 sequences)

SEQ ID NO: 1 - protein, 225 aa, synthetic construct
MLRERALRAAPHVLRYHEDWEPVAEPLADAYAEVAPWLLRDRTEPAPERFFRQLELPLRDKRVCIVGIDPYPEGATGVPFESPDFSKKTARALAAAAARAAEHGGCRRVSAYRNYDFRGVQGVLAWNYYLSCRRGETKSHAMHWERIARMLLAHIARFVRVFYFLGRSDFGGVRAKLTAPVTLLVGYHPAARGGQFESERTLEILNVLLELHGLAPVDWQGFVPL

SEQ ID NO: 2 - protein, 218 aa, synthetic construct
MNSVTVSHAPYTITYHDDWEPVMSQLVEFYNEVASWLLRDETSPIPDKFFIQLKQPLRNKRVCVCGIDPYPKDGTGVPFESPNFTKKSIKEIASSISRLTGVIDYKGYNLNIIDGVIPWNYYLSCKLGETKSHAIYWDKISKLLLQHITKHVSVLYCLGKTDFSNIRAKLESPVTTIVGYHPAARDRQFEKDRSFEIINVLLELDNKAPINWAQGFIY